SEQUENCE LISTING (44 sequences)

SEQ ID NO: 1 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 2 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 3 - protein, 760 aa, Macaca mulatta
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 4 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 5 - protein, 197 aa, Artificial Sequence
  Synthetic Polypeptide
FVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 6 - protein, 7 aa, Artificial Sequence
  Synthetic Polypeptide
ASSLNIA

SEQ ID NO: 7 - protein, 12 aa, Artificial Sequence
  Synthetic Polypeptide
SKTFNTHPQSTP

SEQ ID NO: 8 - protein, 12 aa, Artificial Sequence
  Synthetic Polypeptide
TARGEHKEEELI

SEQ ID NO: 9 - protein, 9 aa, Artificial Sequence
  Synthetic Polypeptide
CQAQGQLVC

SEQ ID NO: 10 - protein, 9 aa, Artificial Sequence
  Synthetic Polypeptide
CSERSMNFC

SEQ ID NO: 11 - protein, 9 aa, Artificial Sequence
  Synthetic Polypeptide
CPKTRRVPC

SEQ ID NO: 12 - protein, 20 aa, Artificial Sequence
  Synthetic Polypeptide
WLSEAGPVVTVRALRGTGSW

SEQ ID NO: 13 - protein, 9 aa, Artificial Sequence
  Synthetic Polypeptide
CMQHSMRVC

SEQ ID NO: 14 - protein, 7 aa, Artificial Sequence
  Synthetic Polypeptide
DDTRHWG

SEQ ID NO: 15 - protein, 4 aa, Artificial Sequence
  Synthetic Polypeptide
  misc_feature (3)..(3): Xaa can be any naturally occurring amino acid
LPXT

SEQ ID NO: 16 - protein, 5 aa, Artificial Sequence
  Synthetic Polypeptide
  misc_feature (3)..(3): Xaa can be any naturally occurring amino acid
LPXTG

SEQ ID NO: 17 - protein, 5 aa, Artificial Sequence
  Synthetic Polypeptide
SYWMH

SEQ ID NO: 18 - protein, 17 aa, Artificial Sequence
  Synthetic Polypeptide
EINPTNGRTNYIEKFKS

SEQ ID NO: 19 - protein, 7 aa, Artificial Sequence
  Synthetic Polypeptide
GTRAYHY

SEQ ID NO: 20 - protein, 11 aa, Artificial Sequence
  Synthetic Polypeptide
RASDNLYSNLA

SEQ ID NO: 21 - protein, 7 aa, Artificial Sequence
  Synthetic Polypeptide
DATNLAD

SEQ ID NO: 22 - protein, 9 aa, Artificial Sequence
  Synthetic Polypeptide
QHFWGTPLT

SEQ ID NO: 23 - protein, 7 aa, Artificial Sequence
  Synthetic Polypeptide
GYTFTSY

SEQ ID NO: 24 - protein, 6 aa, Artificial Sequence
  Synthetic Polypeptide
NPTNGR

SEQ ID NO: 25 - protein, 6 aa, Artificial Sequence
  Synthetic Polypeptide
TSYWMH

SEQ ID NO: 26 - protein, 13 aa, Artificial Sequence
  Synthetic Polypeptide
WIGEINPTNGRTN

SEQ ID NO: 27 - protein, 6 aa, Artificial Sequence
  Synthetic Polypeptide
ARGTRA

SEQ ID NO: 28 - protein, 7 aa, Artificial Sequence
  Synthetic Polypeptide
YSNLAWY

SEQ ID NO: 29 - protein, 10 aa, Artificial Sequence
  Synthetic Polypeptide
LLVYDATNLA

SEQ ID NO: 30 - protein, 8 aa, Artificial Sequence
  Synthetic Polypeptide
QHFWGTPL

SEQ ID NO: 31 - protein, 9 aa, Artificial Sequence
  Synthetic Polypeptide
QHFAGTPLT

SEQ ID NO: 32 - protein, 8 aa, Artificial Sequence
  Synthetic Polypeptide
QHFAGTPL

SEQ ID NO: 33 - protein, 116 aa, Artificial Sequence
  Synthetic Polypeptide
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSS

SEQ ID NO: 34 - protein, 107 aa, Artificial Sequence
  Synthetic Polypeptide
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELK

SEQ ID NO: 35 - protein, 116 aa, Artificial Sequence
  Synthetic Polypeptide
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 36 - protein, 107 aa, Artificial Sequence
  Synthetic Polypeptide
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 37 - protein, 330 aa, Artificial Sequence [sequence omitted]

SEQ ID NO: 38 - protein, 107 aa, Artificial Sequence [sequence omitted]

SEQ ID NO: 39 - protein, 446 aa, Artificial Sequence
  Synthetic Polypeptide
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, Artificial Sequence
  Synthetic Polypeptide
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 446 aa, Artificial Sequence
  Synthetic Polypeptide
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, Artificial Sequence
  Synthetic Polypeptide
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 226 aa, Artificial Sequence
  Synthetic Polypeptide
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 44 - protein, 226 aa, Artificial Sequence
  Synthetic Polypeptide
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

(19) United States
(12) Patent Application Publication
(10) Pub. No.: US 2022/0306685 A1
(43) Pub. Date: Sep. 29, 2022

(54) METHODS OF PREPARING PROTEIN-OLIGONUCLEOTIDE COMPLEXES

(71) Applicant: Dyne Therapeutics, Inc., Waltham, MA (US)

(72) Inventors: Timothy Weeden, Waltham, MA (US); Sean Spring, Waltham, MA (US)

(73) Assignee: Dyne Therapeutics, Inc., Waltham, MA (US)

(21) Appl. No.: 17/616,870

(22) PCT Filed: Jun. 5, 2020
(86) PCT No.: PCT/US2020/036307

§ 371 Date: Dec. 6, 2021

Related U.S. Application Data
(60) Provisional application No. 62/858,964, filed on Jun. 7, 2019.
(60) Provisional application No. 62/992,187, filed on Mar. 20, 2020.

(51) Int. Cl. C07K 1/20; C07K 16/28; A61K 47/68; C12N 15/113; C07K 1/16

(52) U.S. Cl. CPC C07K 1/20; C07K 16/2881; A61K 47/6807; A61K 47/6849; C12N 15/1137; C07K 1/165; C12N 2310/11; C12N 2310/3513; C07K 2317/55

(57) ABSTRACT

Aspects of the disclosure relate to methods of purifying complexes comprising a protein (e.g., antibody) covalently linked to an oligonucleotide. In some embodiments, complexes comprising a protein covalently linked to an oligonucleotide are purified and isolated from unlinked oligonucleotide using an mixed-mode resin that comprises positively-charged metal sites and negatively charged ionic sites, e.g., hydroxyapatite resin. In some embodiments, complexes comprising a protein covalently linked to an oligonucleotide are purified from a mixture comprising the complexes, unlinked protein, and unlinked oligonucleotide using a purification step involving hydrophobic interaction chromatography resin followed by a purification step involving mixed-mode resin.

### RELATED APPLICATIONS

This application claims the benefit under 35 U.S.C. § 119(e) to U.S. Provisional Application No. 62/858,964, filed Jun. 7, 2019, entitled “METHODS OF PREPARING PROTEIN-OLIGONUCLEOTIDE COMPLEXES,” and to U.S. Provisional Application No. 62/992,187, filed Mar. 20, 2020, entitled “METHODS OF PREPARING PROTEIN-OLIGONUCLEOTIDE COMPLEXES,” the entire contents of each of which are incorporated herein by reference.

### FIELD OF THE INVENTION

The present application relates to methods of purifying complexes (e.g., protein-oligonucleotide conjugates).

### REFERENCE TO THE SEQUENCE LISTING

The present application is being filed along with a Sequence Listing in electronic format. The Sequence Listing is provided as a file entitled D082470010WO00-SEQ.txt created on Jun. 5, 2020 which is 56 kb in size. The information in electronic format of the sequence listing is incorporated herein by reference in its entirety.

### BACKGROUND

In recent years, several oligonucleotides (e.g., antisense oligonucleotides) have been developed to combat tissue- or cell-specific diseases (e.g., muscle-specific diseases, e.g., various forms of muscular dystrophy). It has, nonetheless, proven challenging to effectively deliver these oligonucleotides to their desired tissues or cells.

### SUMMARY

Complexes comprising tissue- or cell-specific proteins (e.g., antibodies) covalently linked to therapeutic oligonucleotides offer excellent opportunities for delivery of said therapeutic oligonucleotides. However, purification and isolation of said complexes away from excess protein and oligonucleotide presents challenges. Herein, the present disclosure provides methods of preparing complexes comprising protein covalently linked to oligonucleotides that separate out unconjugated oligonucleotides (e.g., single stranded oligonucleotides) and proteins (e.g., antibodies) from the complexes.

According to some aspects, the disclosure provides methods of processing (e.g., purifying) complexes (e.g., antibodies-oligonucleotide conjugates). In some embodiments, the processed complexes target muscle cells for purposes of delivering molecular payloads e.g., oligonucleotides) to those cells. In some embodiments, the processed complexes of the present disclosure facilitate muscle-specific delivery of molecular payloads e.g., oligonucleotides) that target muscle disease alleles. For example, in some embodiments, processed complexes provided herein are particularly useful for delivering molecular payloads e.g., oligonucleotides) that modulate the expression or activity of a gene in a subject having or suspected of having a muscle disease associated with the gene (e.g., a gene/disease of Table 1).

Some aspects of the present disclosure provide methods of isolating a complex or plurality of complexes each comprising an antibody covalently linked to one or more oligonucleotides, the method comprising:

(i) contacting a mixture comprising the complexes and unlinked antibodies with a hydrophobic resin under conditions in which the complexes but not the unlinked antibodies adsorb to the hydrophobic resin, thus separating the unlinked antibodies from the complexes adsorbed to the hydrophobic resin; and

(ii) eluting the complexes from the hydrophobic resin under conditions in which the complex dissociate from the hydrophobic resin.

In some embodiments, the conditions in step (i) comprise a conductivity of at least 70 mS/cm, and/or the conditions in step (ii) comprises a conductivity of 10-70 mS/cm.

In some embodiments, the conditions in step (i) and or step (ii) are achieved using an anti-chaotropic salt, optionally wherein the anti-chaotropic salt is ammonium sulfate. In some embodiments, the mixture in step (i) further comprises at least 500 mM of ammonium sulfate, optionally wherein the mixture in step (i) further comprises 500 mM-1 M ammonium sulfate.

In some embodiments, comprising washing the hydrophobic resin between step (i) and step (ii) with a solution comprising at least 500 mM of ammonium sulfate.

In some embodiments, step (ii) comprises applying an elution solution comprising up to 200 mM of chloride ions and up to 100 mM of ammonium sulfate to the hydrophobic resin to elute the complexes. In some embodiments, the elution solution does not contain ammonium sulfate. In some embodiments, the elution solution is PBS. In some embodiments, the elution solution comprises up to 25 mM chloride ions.

In some embodiments, step (ii) comprises applying a gradually decreasing concentration of ammonium sulfate to the hydrophobic resin to elute the complexes, optionally wherein the concentration of ammonium sulfate decreases from at least 500 mM to less than 100 mM. In some embodiments, the gradually decreasing concentration of ammonium sulfate is applied over 5-12 column volumes (CVs), optionally 6-8 CVs.

In some embodiments, the mixture in step (i) further comprises unlinked oligonucleotides, optionally wherein the oligonucleotides adsorb to the hydrophobic resin in step (i) and are eluted in step (ii) with the complexes.

In some embodiments, the antibody is a full length IgG, a Fab fragment, a Fab′ fragment, a F(ab′)2 fragment, a scFv, or a Fv fragment. In some embodiments, the antibody is an anti-transferrin receptor antibody.

In some embodiments, the oligonucleotide is single stranded. In some embodiments, the oligonucleotide is an antisense oligonucleotide, optionally a gapmer or a phosphorodiamidate morpholino oligomer (PMO). In some embodiments, the oligonucleotide is one strand of a double stranded oligonucleotide, optionally wherein the double stranded oligonucleotide is a siRNA, and optionally wherein the one strand is the sense strand of the siRNA. In some embodiments, the oligonucleotide comprises at least one modified internucleotide linkage, optionally wherein the at least one modified internucleotide linkage is a phosphorothioate linkage. In some embodiments, the oligonucleotide comprises one or more modified nucleotides, optionally wherein the modified nucleotide comprises 2′-O-methoxyethylribose (MOE), locked nucleic acid (LNA), a 2′-fluoro modification, or a morpholino modification. In some embodiments, the oligonucleotide is 10-50 nucleotides in length, optionally 15-25 nucleotides in length. In some embodiments, the antibody is covalently linked to the 5′ of the oligonucleotide. In some embodiments, the antibody is covalently linked to the 3′ of the oligonucleotide.

In some embodiments, the antibody is covalently linked to the oligonucleotide via a linker, optionally a Val-cit linker.

In some embodiments, the complexes eluted in step (ii) comprises an antibody covalently linked to 1, 2, or 3 oligonucleotides.

In some embodiments, the hydrophobic resin comprises a hydrophobic moiety selected butyl, t-butyl, phenyl, ether, amide, or propyl groups.

In some embodiments, the hydrophobic resin is equilibrated prior to step (i), optionally equilibrated with a solution comprising at least 500 mM of ammonium sulfate.

In some embodiments, the eluent obtained from step (ii) comprises undetectable levels of unlinked antibodies.

In some embodiments, the method further comprises isolating the complexes from the unlinked oligonucleotides.

Other aspects of the present disclosure provide methods of isolating a complex or plurality of complexes each comprising an antibody covalently linked to one or more oligonucleotides, the method comprising:

(i) contacting a mixture comprising the complexes and unlinked oligonucleotides with a mixed-mode resin that comprises positively-charged metal sites and negatively charged ionic sites, under conditions in which the complexes adsorb to the mixed-mode resin, and

(ii) eluting the complexes from the mixed-mode resin under conditions in which the complexes dissociate from the mixed-mode resin.

In some embodiments, the mixed-mode resin is an apatite resin. In some embodiments, the apatite resin is a hydroxyapatite resin, a ceramic hydroxyapatite resin, a hydroxyfluoroapatite resin, a fluoroapatite resin, or a chlorapatite resin.

In some embodiments, the mixture in step (i) further comprises up to 20 mM phosphate ions and/or up to 30 mM chloride ions, optionally wherein the mixture in step (i) further comprises up to 10 mM phosphate ions and/or up to 25 mM chloride ions. In some embodiments, the unlinked oligonucleotide does not adsorb to the mixed-mode resin in step (i).

In some embodiments, the mixture in step (i) further comprises up to 5 mM phosphate ions and/or up to 10 mM chloride ions, optionally wherein the mixture in step (i) further comprises up to 3 mM phosphate ions and/or up to 8 mM chloride ions. In some embodiments, some or all of the unlinked oligonucleotide adsorb to the mixed-mode resin in step (i). In some embodiments, the method further comprises washing the mixed-mode resin between step (i) and step (ii) with a solution comprising up to 20 mM phosphate ions and/or up to 30 mM chloride ions, optionally wherein the solution comprises up to 10 mM phosphate ions and/or up to 25 mM chloride ions.

In some embodiments, step (ii) comprises applying an elution solution comprising at least 30 mM phosphate ions and/or at least 50 mM chloride ions to the mixed-mode resin to elute the complexes, optionally wherein the elution solution comprises at least 100 mM phosphate ions and/or at least 100 mM chloride ions.

In some embodiments, the antibody is a full length IgG, a Fab fragment, a Fab′ fragment, a F(ab′)2 fragment, a scFv, or a Fv fragment. In some embodiments, the antibody is an anti-transferrin receptor antibody.

In some embodiments, the oligonucleotide is single stranded. In some embodiments, the oligonucleotide is an antisense oligonucleotide, optionally a gapmer or a phosphorodiamidate morpholino oligomer (PMO). In some embodiments, the oligonucleotide is one strand of a double stranded oligonucleotide, optionally wherein the double stranded oligonucleotide is a siRNA, and optionally wherein the one strand is the sense strand of the siRNA. In some embodiments, the oligonucleotide comprises at least one modified internucleotide linkage, optionally wherein the at least one modified internucleotide linkage is a phosphorothioate linkage. In some embodiments, the oligonucleotide comprises one or more modified nucleotides, optionally wherein the modified nucleotide comprises 2′-O-methoxyethylribose (MOE), locked nucleic acid (LNA), a 2′-fluoro modification, or a morpholino modification. In some embodiments, the oligonucleotide is 10-50 nucleotides in length, optionally 15-25 nucleotides in length. In some embodiments, the antibody is covalently linked to the 5′ of the oligonucleotide. In some embodiments, the antibody is covalently linked to the 3′ of the oligonucleotide.

In some embodiments, the antibody is covalently linked to the oligonucleotide via a linker, optionally a Val-cit linker.

In some embodiments, the complexes eluted in step (ii) comprise an antibody covalently linked to 1, 2, or 3 oligonucleotides.

In some embodiments, the eluent obtained from step (ii) comprises undetectable levels of unlinked oligonucleotide.

In some embodiments, the mixture in step (i) was isolated from a hydrophobic interaction chromatography resin prior to step (i).

Further provided herein are methods of isolating a complex or plurality of complexes each comprising an antibody covalently linked to one or more oligonucleotides, the method comprising:

(i) contacting a first mixture comprising the complexes, unlinked antibodies, and unlinked oligonucleotides with a hydrophobic resin under conditions in which the complexes and the unlinked oligonucleotides but not the unlinked antibodies adsorb to the hydrophobic resin, thus separating the unlinked antibodies from the complexes and the unlinked oligonucleotides adsorbed to the hydrophobic resin; and

(ii) obtaining a second mixture comprising the complexes and the unlinked oligonucleotides by eluting the complexes and the unlinked oligonucleotides from the hydrophobic resin under conditions in which the complexes dissociate from the hydrophogic resin;

(iii) contacting the second mixture obtained in step (ii) with a mixed-mode resin that comprises positively-charged metal sites and negatively charged ionic sites, under conditions in which the complexes adsorb to the mixed-mode resin, and

(iv) eluting the complexes from the mixed-mode resin under conditions in which the complexes dissociate from the mixed-mode resin.

In some embodiments, the hydrophobic resin comprises a hydrophobic moiety selected from butyl, t-butyl, phenyl, ether, amide, or propyl groups. In some embodiments, the mixed-mode resin is an apatite resin, optionally wherein the apatite resin is a hydroxyapatite resin, a ceramic hydroxyapatite resin, a hydroxyfluoroapatite resin, a fluoroapatite resin, or a chlorapatite resin.

In some embodiments, the conditions in step (i) comprise a conductivity of at least 70 mS/cm, and/or the conditions in step (ii) comprises a conductivity of 10-70 mS/cm. In some embodiments, the conditions in step (i) and or step (ii) are achieved using an anti-chaotropic salt, optionally wherein the anti-chaotropic salt is ammonium sulfate. In some embodiments, the hydrophobic resin is equilibrated prior to step (i), optionally equilibrated with a solution comprising at least 500 mM of ammonium sulfate. In some embodiments, the mixture in step (i) further comprises at least 500 mM of ammonium sulfate, optionally wherein the mixture in step (i) further comprises 500 mM-1 M of ammonium sulfate. In some embodiments, the method further comprises washing the hydrophobic resin between step (i) and step (ii) with a solution comprising at least 500 mM of ammonium sulfate. In some embodiments, step (ii) comprises applying a first elution solution comprising up to 200 mM of chloride ions and up to 100 mM of ammonium sulfate to the hydrophobic resin to elute the complexes and the unlinked oligonucleotides, optionally wherein the first elution solution does not contain ammonium sulfate. In some embodiments, the first elution solution is PBS, or comprises up to 25 mM chloride ions. In some embodiments, step (ii) comprises applying a gradually decreasing concentration of ammonium sulfate to the hydrophobic resin to elute the complexes and the unlinked oligonucleotides, optionally wherein the concentration of ammonium sulfate decreases from at least 500 mM to less than 100 mM and/or the gradually decreasing concentration of ammonium sulfate is applied over 5-12 column volumes (CVs), optionally 6-8 CVs.

In some embodiments, the second mixture in step (iii) further comprises up to 20 mM phosphate ions and/or up to 30 mM chloride ions, optionally wherein the second mixture in step (iii) further comprises up to 10 mM phosphate ions and/or up to 25 mM chloride ions. In some embodiments, the unlinked oligonucleotide does not adsorb to the mixed-mode resin in step (iii). In some embodiments, the second mixture in step (iii) further comprises up to 5 mM phosphate ions and/or up to 10 mM chloride ions, optionally wherein the second mixture in step (iii) further comprises up to 3 mM phosphate ions and/or up to 8 mM chloride ions. In some embodiments, some or all of the unlinked oligonucleotide adsorb to the mixed-mode resin in step (iii). In some embodiments, the method further comprises washing the mixed-mode resin between step (iii) and step (iv) with a solution comprising up to 20 mM phosphate ions and/or up to 30 mM chloride ions to remove the unlinked oligonucleotide from the mixed mode resin, optionally wherein the solution comprises up to 10 mM phosphate ions and/or up to 25 mM chloride ions. In some embodiments, step (iv) comprises applying a second elution solution comprising at least 30 mM phosphate ions and/or at least 50 mM chloride ions to the mixed-mode resin to elute the complexes, optionally wherein the second elution solution comprises at least 100 mM phosphate ions and/or at least 100 mM chloride.

In some embodiments, the antibody is a full length IgG, a Fab fragment, a Fab′ fragment, a F(ab′)2 fragment, a scFv, or a Fv fragment. In some embodiments, the antibody is an anti-transferrin receptor antibody.

In some embodiments, the oligonucleotide is single stranded. In some embodiments, the oligonucleotide is an antisense oligonucleotide, optionally a gapmer or a phosphorodiamidate morpholino oligomer (PMO). In some embodiments, the oligonucleotide is one strand of a double stranded oligonucleotide, optionally wherein the double stranded oligonucleotide is a siRNA, and optionally wherein the one strand is the sense strand of the siRNA. In some embodiments, the oligonucleotide comprises at least one modified internucleotide linkage, optionally wherein the at least one modified internucleotide linkage is a phosphorothioate linkage. In some embodiments, the oligonucleotide comprises one or more modified nucleotides, optionally wherein the modified nucleotide comprises 2′-O-methoxyethylribose (MOE), locked nucleic acid (LNA), a 2′-fluoro modification, or a morpholino modification. In some embodiments, the oligonucleotide is 10-50 nucleotides in length, optionally 15-25 nucleotides in length. In some embodiments, the antibody is covalently linked to the 5′ of the oligonucleotide. In some embodiments, the antibody is covalently linked to the 3′ of the oligonucleotide.

In some embodiments, the antibody is covalently linked to the oligonucleotide via a linker, optionally a Val-cit linker.

In some embodiments, the complexes eluted in step (iv) comprise an antibody covalently linked to 1, 2, or 3 oligonucleotides.

In some embodiments, the eluent obtained from step (iv) comprises undetectable levels of unlinked oligonucleotide and/or undetectable levels of unlinked antibodies.

In some aspects, methods of processing complexes that comprise a protein covalently linked to one or more oligonucleotides comprise:
(i) separating the complexes from unlinked oligonucleotides by contacting a mixture that comprises the complexes and the unlinked oligonucleotides with a mixed-mode resin that comprises positively-charged metal sites and negatively charged ionic sites, under conditions in which the complexes adsorb to the mixed-mode resin, and
(ii) eluting the unlinked oligonucleotide while the complexes remain adsorbed to the mixed-mode resin.

In some embodiments, the mixed-mode resin is an apatite resin (e.g., a hydroxyapatite resin, a ceramic hydroxyapatite resin, a hydroxyfluoroapatite resin, a fluoroapatite resin, or a chlorapatite resin). In some embodiments, a mixture that comprises the complexes and the unlinked oligonucleotides further comprises at least 1 mM phosphate ions and/or at least 5 mM chloride ions (e.g., 5 mM phosphate ions and 25 mM chloride ions). In some embodiments, the unlinked oligonucleotide is eluted in step (ii) by the addition of a wash solution to the mixed-mode resin, optionally wherein the wash solution comprises 1-50 mM phosphate ions and/or at least 5-50 mM chloride ions (e.g., 5 mM phosphate ions and 25 mM chloride ions).

In some embodiments, the method further comprises step (iii), following step (ii), eluting the plurality of complexes from the mixed-mode resin. In some embodiments, the plurality of complexes are eluted in step (iii) by the addition of an eluent solution to the mixed-mode resin (e.g., an eluent solution comprising at least 5 mM phosphate ions and/or at least 5 mM chloride ions, e.g., 100 mM phosphate ions and 100 mM chloride ions).

In some aspects, methods of processing complexes, wherein each complex comprises a protein covalently linked to one or more oligonucleotides, the method comprises:
(i) contacting a first mixture comprising the complexes, unlinked oligonucleotides, and unlinked proteins with a hydrophobic interaction chromatographic (HIC) resin, under conditions in which the complexes and unlinked oligonucleotides adsorb to the HIC resin;
(ii) eluting the unlinked protein from the HIC resin;
(iii) following step (ii), eluting from the HIC resin a second mixture comprising the complexes and unlinked oligonucleotides;
(iv) contacting the second mixture with an mixed-mode resin that comprises positively-charged metal sites and negatively charged ionic site, under conditions in which the complexes adsorb to the mixed-mode resin;
(v) eluting the unlinked oligonucleotide while the complexes remain adsorbed to the mixed-mode resin; and
(vi) following step (v), eluting the plurality of complexes from the mixed-mode resin.

In some embodiments, the HIC resin comprises butyl, t-butyl, methyl, and/or ethyl functional groups. In some embodiments, the HIC resin is equilibrated prior to step (i), e.g., equilibrated with at least 500 mM ammonium sulfate. In some embodiments, the unlinked protein is eluted in step (ii) by the addition of a HIC wash solution to the HIC resin (e.g., a HIC wash solution comprising at least 500 mM ammonium sulfate). In some embodiments, the complexes and unlinked oligonucleotide are eluted from the HIC resin in step (iii) by the addition of a HIC eluent solution to the HIC resin (e.g., a HIC eluent solution comprising less than 100 mM phosphate ions and/or 100 mM chloride ions, e.g., 5 mM phosphate ions and 25 mM chloride ions.

In some embodiments, the mixed-mode resin is an apatite resin (e.g., a hydroxyapatite resin, a ceramic hydroxyapatite resin, a hydroxyfluoroapatite resin, a fluoroapatite resin, or a chlorapatite resin). In some embodiments, a mixture that comprises the complexes and the unlinked oligonucleotides further comprises at least 1 mM phosphate ions and/or at least 5 mM chloride ions (e.g., 5 mM phosphate ions and 25 mM chloride ions). In some embodiments, the unlinked oligonucleotide is eluted in step (ii) by the addition of a wash solution to the mixed-mode resin, optionally wherein the wash solution comprises 1-50 mM phosphate ions and/or at least 5-50 mM chloride ions (e.g., 5 mM phosphate ions and 25 mM chloride ions).

In some embodiments, the method further comprises step (iii), following step (ii), eluting the plurality of complexes from the mixed-mode resin. In some embodiments, the plurality of complexes are eluted in step (iii) by the addition of an eluent solution to the mixed-mode resin (e.g., an eluent solution comprising at least 5 mM phosphate ions and/or at least 5 mM chloride ions, e.g., 100 mM phosphate ions and 100 mM chloride ions).

In some embodiments, the protein of a complex to be processed is an antibody. The antibody may be a muscle-targeting antibody, such as a muscle-targeting antibody that specifically binds to an extracellular epitope of a transferrin receptor. In some embodiments, the muscle-targeting antibody competes for specific binding to an epitope of a transferrin receptor with an antibody listed in Table 2. An antibody (e.g., a muscle-targeting antibody) may be in the form of a ScFv, a Fab fragment, Fab′ fragment, F(ab′)2 fragment, or Fv fragment.

In some embodiments, the oligonucleotide of a complex to be processed comprises at least one modified internucleotide linkage (e.g., a phosphorothioate linkage). The oligonucleotide may comprise one or more modified nucleotides. In some embodiments, the oligonucleotide is 10-50 nucleotides in length (e.g., 15-25 nucleotides in length). In some embodiments, the oligonucleotide comprises a region of complementarity to gene listed in Table 1 or mRNA encoded therefrom.

In some embodiments, complexes (e.g., following methods of processing described herein) comprise undetectable levels of unlinked protein and/or undetectable levels of unlinked oligonucleotide.

### BRIEF DESCRIPTION OF THE DRAWINGS

FIG. 1 provides an image of an sodium dodecyl sulfate-polyacrylamide gel electrophoresis (non-reducing SDS-PAGE) gel. Recombinantly expressed DTX-A-001 Fab′, an anti-transferrin antibody, following a protein L chromatography purification, is shown in lane 1; DTX-A-001 Fab′, comprises a MW of ˜49 kDa. SeeBlue Plus2 ladder, a reference that comprises proteins of standard masses, is shown in lane 2.

FIG. 2 provides images of SDS-PAGE gels of mixtures of complexes comprising DTX-A-001 Fab′ linked to an antisense oligonucleotide (DMPK ASO), unlinked DMPK ASO, and unlinked DTX-A-001 Fab′ before and after contacting with a hydrophobic interaction chromatography (HIC) resin. Mixtures before and after HIC purification are shown in the innermost lanes; See Blue Plus2 ladder is shown in the outermost lanes.

FIG. 3 is an overlay of three analytical size exclusion chromatography (SEC) experiments demonstrating the purification of complexes comprising DTX-A-001 Fab′ linked to an antisense oligonucleotide (DMPK ASO) from unlinked DMPK ASO. Shown are an unpurified mixture of complex comprising DTX-A-001 Fab′ linked to an antisense oligonucleotide (DMPK ASO) and unlinked DMPK ASO before contacting with a hydroxyapatite (HA) resin (‘HA load’), unlinked DMPK ASO that did not bind to the HA resin (‘HA flow through’), and purified complex (‘HA elute’).

FIG. 4 is a chromatograph showing the isolation of complexes comprising DTX-P-060 covalently linked to DTX-A-012 from a mixture containing the complex, unlinked DTX-P-060 and unlinked DTX-A-012 via hydrophobic interaction chromatography.

FIG. 5 is a chromatograph showing the isolation of complexes comprising DTX-P-060 covalently linked to DTX-A-012 from a mixture containing the complex and unlinked DTX-P-060 via mixed-mode resin (hydroxyapatite) chromatography.

FIG. 6 is a chromatograph showing the isolation of complexes comprising DTX-P-060 covalently linked to an anti-transferrin receptor antibody listed in Table 2 from a mixture containing the complex, unlinked DTX-P-060 and unlinked antibody via hydrophobic interaction chromatography. A shallow gradient (gradient over 8 column volumes) of decreasing ammonium sulfate concentration was used for eluting the complexes, resulting in separation of different DAR species (DAR 1 and DAR 2).

FIG. 7 shows the analysis of the gradient fractions obtained in FIG. 6 using analytical size exclusion chromatography (SEC). Top panel shows an overlay of A280 nm of the pooled fractions from the hydrophobic interaction chromatography. Middle panel shows A280 nm of the flow through from the hydrophobic interaction chromatography. Lower panel shows A280 nm of the shoulder peak from the hydrophobic interaction chromatography.

FIG. 8 is a chromatograph showing the isolation of complexes comprising DTX-P-060 covalently linked to a Fab′ of an anti-transferrin receptor antibody listed in Table 2 from a mixture containing the complex, unlinked DTX-P-060 and unlinked antibody via hydrophobic interaction chromatography. A steep gradient (gradient over 6 column volumes) of decreasing ammonium sulfate concentration was used for eluting the complexes. DAR species are pooled compared to the elution using the shallow gradient in FIG. 6.

FIG. 9 shows the analysis of the gradient fractions obtained in FIG. 8 using analytical size exclusion chromatography (SEC). Top panel shows A280 nm of the pooled peak at a conductivity of 41 mS/cm from the hydrophobic interaction chromatography, which contains >96% of DAR1. Middle panel shows A280 nm of the pooled peak at a conductivity of 33 mS/cm from the hydrophobic interaction chromatography, which contains >55% of DAR2. Lower panel shows A280 nm of the pooled peak at a conductivity of 22 mS/cm from the hydrophobic interaction chromatography, which contains >69% of DAR 2 and some level of DAR 3.

FIG. 10 is a SDS-PAGE analysis of the pooled peaks from FIG. 8.

FIGS. 11A and 11B show the purification of complexes comprising an anti-transferrin receptor Fab′ covalently linked to one or more oligonucleotides from a mixture containing the complexes, unlinked oligonucleotides, and unlinked antibody via hydrophobic interaction chromatography. FIG. 11A shows the purification chromatograph. FIG. 11B shows the analysis of the eluted fractions using SDS-PAGE.

FIGS. 12A and 12B show the purification of complexes comprising an anti-transferrin receptor Fab′ covalently linked to one or more oligonucleotides from a mixture containing the complexes, unlinked oligonucleotides, and unlinked antibody via hydrophobic interaction chromatography. FIG. 12A shows the purification chromatograph. FIG. 12B shows the analysis of the eluted fractions using SDS-PAGE.

FIGS. 13A and 13B show the purification of complexes comprising the RI7217 antibody (full length IgG) covalently linked to DTX-P-60 from a mixture containing the complexes, unlinked DTX-P-60, and unlinked antibody via hydrophobic interaction chromatography. FIG. 11A shows the purification chromatograph. FIG. 11B shows the analysis of the eluted fractions using SDS-PAGE.

FIG. 14 is a chromatograph showing further isolation of the complex described in FIGS. 13A-13B from unlinked oligonucleotides with mixed-mode resin (FA resin).

FIG. 15. shows the analysis of the fractions obtained in FIG. 14 using analytical size exclusion chromatography (SEC). Top panel shows that unlinked oligonucleotides dissociated from the mixed-mode resin at a phosphate ion concentration of 10 mM. Bottom panel shows the complexes eluted at a phosphate ion concentration of 100 mM.

### DETAILED DESCRIPTION OF CERTAIN EMBODIMENTS

As described herein, the present disclosure provides methods of purifying complexes, e.g., complexes comprising proteins (e.g., muscle-targeting agents (e.g., an antibody) covalently linked to molecular payloads (e.g., oligonucleotides)). In some embodiments, a complex or plurality of complexes comprising a protein (e.g., an antibody) covalently linked to an oligonucleotide is purified from a mixture comprising said complex and unlinked (e.g., excess) proteins using a hydrophobic resin. In some embodiments, a complex or plurality of complexes comprising a protein (e.g., an antibody) covalently linked to an oligonucleotide is purified from a mixture comprising said complex and unlinked (e.g., excess) oligonucleotide using a mixed-mode resin that comprises positively-charged metal sites and negatively charged ionic sites (e.g., hydroxyapatite resin, ceramic hydroxyapatite resin, hydroxyfluoroapatite resin, fluoroapatite resin, chlorapatite resin). In some embodiments, a complex or plurality of complexes comprising a protein (e.g., an antibody) covalently linked to an oligonucleotide is purified from a mixture comprising said complex, unlinked (e.g., excess) oligonucleotide, and unlinked (e.g., excess protein) using a first purification step involving a hydrophilic interaction chromatographic resin and a second purification step involving a mixed-mode resin that comprises positively-charged metal sites and negatively charged ionic sites (e.g., hydroxyapatite resin, ceramic hydroxyapatite resin, hydroxyfluoroapatite resin, fluoroapatite resin, chlorapatite resin).

In some embodiments, the complexes being purified are particularly useful for delivering molecular payloads (e.g., oligonucleotides) that modulate expression or activity of target genes in muscle cells, e.g., in a subject having or suspected of having a muscle disease. For example, in some embodiments, complexes are useful for treating subjects having rare muscle diseases, including Pompe disease, Centronuclear myopathy, Fibrodysplasia Ossificans Progressiva, Friedreich's ataxia, or Duchenne muscular dystrophy. In some embodiments, depending on the condition to be treated, different oligonucleotides may be used in such complexes.

Further aspects of the disclosure, including a description of defined terms, are provided below.

### I. Definitions

Administering: As used herein, the terms “administering” or “administration” means to provide a complex to a subject in a manner that is physiologically and/or pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody, e.g., a full-length IgG. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (A), epsilon (E), gamma (γ) or mu (p) heavy chain. In some embodiments, the heavy chain of an antibody described herein can comprise a human alpha (a), delta (A), epsilon (e), gamma (γ) or mu (p) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Example linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDR-grafted antibody: The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, N.Y., 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferring receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Disease allele: As used herein, the term “disease allele” refers to any one of alternative forms (e.g., mutant forms) of a gene for which the allele is correlated with and/or directly or indirectly contributes to, or causes, disease. A disease allele may comprise gene alterations including, but not limited to, insertions (e.g., disease-associated repeats described below), deletions, missense mutations, nonsense mutations and splice-site mutations relative to a wild-type (non-disease) allele. In some embodiments, a disease allele has a loss-of-function mutation. In some embodiments, a disease allele has a gain-of-function mutation. In some embodiments, a disease allele encodes an activating mutation (e.g., encodes a protein that is constitutively active). In some embodiments, a disease allele is a recessive allele having a recessive phenotype. In some embodiments, a disease allele is a dominant allele having a dominant phenotype.

Disease-associated-repeat: As used herein, the term “disease-associated-repeat” refers to a repeated nucleotide sequence at a genomic location for which the number of units of the repeated nucleotide sequence is correlated with and/or directly or indirectly contributes to, or causes, genetic disease. Each repeating unit of a disease associated repeat may be 2, 3, 4, 5 or more nucleotides in length. For example, in some embodiments, a disease associated repeat is a dinucleotide repeat. In some embodiments, a disease associated repeat is a trinucleotide repeat. In some embodiments, a disease associated repeat is a tetranucleotide repeat. In some embodiments, a disease associated repeat is a pentanucleotide repeat. In some embodiments, embodiments, the disease-associated-repeat comprises CAG repeats, CTG repeats, CUG repeats, CGG repeats, CCTG repeats, or a nucleotide complement of any thereof. In some embodiments, a disease-associated-repeat is in a non-coding portion of a gene. However, in some embodiments, a disease-associated-repeat is in a coding region of a gene. In some embodiments, a disease-associated-repeat is expanded from a normal state to a length that directly or indirectly contributes to, or causes, genetic disease. In some embodiments, a disease-associated-repeat is in RNA (e.g., an RNA transcript). In some embodiments, a disease-associated-repeat is in DNA (e.g., a chromosome, a plasmid). In some embodiments, a disease-associated-repeat is expanded in a chromosome of a germline cell. In some embodiments, a disease-associated-repeat is expanded in a chromosome of a somatic cell. In some embodiments, a disease-associated-repeat is expanded to a number of repeating units that is associated with congenital onset of disease. In some embodiments, a disease-associated-repeat is expanded to a number of repeating units that is associated with childhood onset of disease. In some embodiments, a disease-associated-repeat is expanded to a number of repeating units that is associated with adult onset of disease.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Hydrophobic interaction chromatographic resin: As used herein, the term “hydrophobic interaction chromatographic resin” or “HIC resin” refers to a chromatographic resin or material that functions in use of purification, separation, and/or isolation of molecules based on hydrophobic interactions between the resin and the molecule (e.g., protein). In some embodiments, HIC resins comprise hydrophobic functional groups, e.g. silica bonded with hydrophobic functional groups. In some embodiments, hydrophobic functional groups of HIC resins are butyl, t-butyl, phenyl, ether, amide, or propyl groups. In some embodiments, hydrophobic functional groups of HIC resins are straight chain alkyl groups or aryl groups.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or an extracellular domain, which may optionally further comprise a ligand-binding domain. In some embodiments, a cell surface receptor becomes internalized by a cell after ligand binding. In some embodiments, a ligand may be a muscle-targeting protein or a muscle-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Mixed-mode resin: As used herein, the term “mixed-mode resin” refers to a chromatographic resin or material for use in purification, separation, and/or isolation of biomolecules that comprises positively-charged metal sites and negatively charged ionic sites. In some embodiments, the metal sites comprise calcium. In some embodiments, the negatively charged ionic sites comprise phosphate, sulfate, fluoride, or chloride. In some embodiments, the metal sites comprise calcium and the negatively charged ionic sites comprise phosphate, and optionally sulfate, fluoride, or chloride. In some embodiments, a mixed-mode resin is an apatite resin. In some embodiments, an apatite resin is hydroxyapatite resin, ceramic hydroxyapatite resin, hydroxyfluoroapatite resin, fluoroapatite resin, or chlorapatite resin. In some embodiments, apatite resin comprises minerals of the formula: Ca₁₀(PO₄)₆(OH)₂. In some embodiments, apatite resin comprises minerals of the formula: Ca₁₀(PO₄)₆F₂. In some embodiments, apatite resin comprises minerals of the formula: Ca₁₀(PO₄)₆Cl₂.

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide, e.g., an oligonucleotide that comprises a strand having a region of complementarity to a target gene.

Muscle Disease Gene: As used herein, the term “muscle disease gene” refers to a gene having a least one disease allele correlated with and/or directly or indirectly contributing to, or causing, a muscle disease. In some embodiments, the muscle disease is a rare disease, e.g., as defined by the Genetic and Rare Diseases Information Center (GARD), which is a program of the National Center for Advancing Translational Sciences (NCATS). In some embodiments, the muscle disease is a rare disease that is characterized as affecting fewer than 200,000 people. In some embodiments, the muscle disease is a single-gene disease. In some embodiments, a muscle disease gene is a gene listed in Table 1.

Muscle-targeting agent: As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload. In some embodiments, the muscle-targeting agent is a muscle targeting protein (e.g., an antibody)

Muscle-targeting antibody: As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidite morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleotides (e.g. 2′-O-methyl sugar modifications, purine or pyrimidine modifications). In some embodiments, an oligonucleotide may comprise one or more modified internucleotide linkage. In some embodiments, an oligonucleotide may comprise one or more phosphorothioate linkages, which may be in the Rp or Sp stereochemical conformation.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of a oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a muscle disease (e.g., any of the diseases provided in Table 1).

Transferrin receptor: As used herein, the term, “transferrin receptor” (also known as TFRC, CD71, p90, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

Unlinked oligonucleotide: As used herein, the term “unlinked oligonucleotide” refers to free oligonucleotide or excess oligonucleotide that is present in solution, e.g., following a conjugation reaction to generate complexes comprising protein linked to oligonucleotide.

Unlinked protein: As used herein, the term “unlinked protein” refers to free protein or excess protein (e.g., free antibody or excess antibody) that is present in solution, e.g., following a conjugation reaction to generate complexes comprising protein linked to oligonucleotide.

### II. Methods of Purification

Provided herein are methods of purifying or isolating complexes that comprise a protein, e.g. an antibody, covalently linked to one or more oligonucleotide (e.g., a single stranded oligonucleotide). In some aspects, the present disclosure provide methods of isolating a complex or plurality of complexes comprising an antibody covalently linked to one or more oligonucleotides from a mixture comprising the complexes, unlinked antibodies, and unlinked oligonucleotides using one or more steps of adsorption chromatography. In some embodiments, the adsorption chromatography steps comprise hydrophobic interaction chromatography (HIC) and mixed-mode resin (e.g., apatide resin) chromatography. In some aspects, the present disclosure provides methods of separating the complexes from unlinked antibodies, e.g., via hydrophobic interaction chromatography (HIC). In some aspects, the present disclosure provides methods of separating the complexes from unlinked oligonucleotides, e.g., via mixed-mode resin (e.g., apatite resin) chromatography. In some embodiments, the methods of purifying a complex or plurality of complexes described herein involve isolating the complexes and the unlinked oligonucleotides by removing the unlinked antibodies by hydrophobic interaction chromatographic (HIC), and isolating the complexes from unlinked oligonucleotide from by mixed-mode resin chromatography.

In some aspects, the methods of purifying a complex or plurality of complexes described herein involving contacting a mixture comprising the complex or plurality of complexes and unlinked proteins (e.g., unlinked antibodies) with a hydrophobic resin, removing the unlinked antibodies that do not adsorb to the hydrophobic resin, and eluting the adsorbed complexes from the hydrophobic resin. In some embodiments, the hydrophobic resin comprises a hydrophobic moiety selected from butyl, t-butyl, phenyl, ether, amide, or propyl groups.

In some aspects, the methods of purifying a complex or plurality of complexes described herein involving contacting a mixture comprising the complex or plurality of complexes and unlinked oligonucleotide with a mixed-mode resin comprising positively-charged metal sites and negatively charged ionic sites, removing the unlinked oligonucleotides, and eluting the adsorbed complexes from the mixed-mode resin. In some embodiments, the mixed-mode resin is an apatite resin. In some embodiments, an apatite resin is a hydroxyapatite resin, a ceramic hydroxyapatite resin, a hydroxyfluoroapatite resin, a fluoroapatite resin, or a chlorapatite resin.

In some embodiments, complexes are substantially purified away from unlinked oligonucleotide and unlinked protein. In some embodiments, compositions of complexes following purification from unlinked oligonucleotide and/or unlinked protein using the methods described herein do not comprise any detectable levels of unlinked oligonucleotide or unlinked protein.

In some embodiments, the methods described herein are suitable for isolating complexes comprising an antibody covalently linked to one or more oligonucleotides. In some embodiments, the antibody may be a full-length IgG, a Fab fragment, a Fab′ fragment, a F(ab′)2 fragment, a scFv, or a Fv fragment. The specific antibody sequences do not affect the purification outcome. In some embodiments, the antibody is an anti-transferrin receptor antibody (e.g., any of the anti-transferrin receptor antibodies listed in Table 2) or any antigen binding fragments thereof (e.g., a Fab fragment, a Fab′ fragment, a F(ab′)2 fragment, a scFv, or a Fv fragment)

In some embodiments, the oligonucleotide is a single stranded oligonucleotide. In some embodiments, the single stranded oligonucleotide is an antisense oligonucleotide. In some embodiments, the antisense oligo nucleotide is a gapmer or a phosphorodiamidate Morpholino oligomer (PMO). In some embodiments, the antibody is covalently linked to the 5′ end of the single stranded oligonucleotide. In some embodiments, the antibody is covalently linked to the 3′ end of the single stranded oligonucleotide. In some embodiments, the antibody is covalently linked to the 5′ end of an antisense oligonucleotide (e.g., gapmer or PMO).

In some embodiments, the single stranded oligonucleotide is one strand of a double stranded oligonucleotide. The conditions of hydrophobic interaction chromatography does not allow annealing of the two strands of a double stranded oligonucleotide. However, one strand of the double stranded oligonucleotide can be covalently conjugated to the antibody and the complexes can be isolated using the method described herein, followed by annealing of the other strand of the double stranded oligonucleotide. In some embodiments, the double stranded oligonucleotide is a siRNA and the sense strand is covalently linked to the antibody (e.g., at the 3′ end or at the 5′ end). In some embodiments, the complexes purified using the methods described herein comprise an antibody covalently linked to the 3′ end of the sense strand of a siRNA. The antisense strand of the siRNA can be annealed to the sense strand post purification.

In some embodiments, the oligonucleotide comprises at least one modified internucleotide linkage. In some embodiments, the at least one modified internucleotide linkage is a phosphorothioate linkage. In some embodiments, the oligonucleotide comprises one or more modified nucleotides. In some embodiments, the modified nucleotide comprises 2′-O-methoxyethylribose (MOE), locked nucleic acid (LNA), a 2′-fluoro modification, or a morpholino modification.

In some embodiments, the oligonucleotide is a single stranded oligonucleotide (e.g., an antisense oligonucleotide) comprising a modified nucleotide comprising 2′-O-methoxyethylribose (MOE), locked nucleic acid (LNA), a 2′-fluoro modification, or a morpholino modification. In some embodiments, the antisense oligonucleotide is a gapmer comprising a modified nucleotide comprising 2′-O-methoxyethylribose (MOE), locked nucleic acid (LNA), or a 2′-fluoro modification. In some embodiments, the antisense oligonucleotide is a phosphorodiamidate Morpholino oligomer (PMO). The antisense oligonucleotide may comprise more than one type of modifications described herein, e.g., having MOE and 2′-fluoro modifications.

In some embodiments, the oligonucleotide is a single stranded oligonucleotide (e.g., one strand of a double stranded RNA such as siRNA) comprising a modified nucleotide comprising 2′-O-methoxyethylribose (MOE), locked nucleic acid (LNA), or a 2′-fluoro modification. In some embodiments, the oligonucleotide is the sense strand of a siRNA comprising a modified nucleotide comprising 2′-O-methoxyethylribose (MOE), locked nucleic acid (LNA), or a 2′-fluoro modification. In some embodiments, the oligonucleotide is the antisense strand of a siRNA comprising a modified nucleotide comprising 2′-O-methoxyethylribose (MOE), locked nucleic acid (LNA), or a 2′-fluoro modification. The sense and antisense strand of the siRNA may comprises the same types or different types of modifications described herein. One or both strands of the siRNA may comprise more than one type of modifications described herein, e.g., having MOE and 2′-fluoro modifications.

In some embodiments, the oligonucleotide is 10-50 (e.g., 10-50, 10-40, 10-30, 10-20, 20-50, 20-40, 20-30, 30-50, 30-40, or 40-50 nucleotides in length), In some embodiments, the oligonucleotide is 15-25 (e.g., 10-25, 10-20, 10-25, 15-25, 15-20, or 20-25) nucleotides in length.

In some embodiments, the oligonucleotide is covalently linked to the antibody via a lysine or a cysteine. In some embodiments, the oligonucleotide is covalently linked to the antibody via a linker (e.g., a linker that comprises a Val-cit linker).

A. Removal of Unlinked Protein (e.g., Antibodies) from a Mixture of Complex and Unlinked Oligonucleotide

It was shown herein that the use of HIC resins are effective in purifying a mixture of complex and unlinked oligonucleotide away from unlinked protein (e.g., unlinked antibodies). In some aspects, the present disclosure provide method of isolating a complex or plurality of complexes each comprising a protein (e.g., an antibody) covalently linked to one or more oligonucleotides, the method comprising: (i) contacting a mixture comprising the complexes and unlinked proteins (e.g., antibodies) with a hydrophobic resin under conditions (e.g., pH, ionic strength, conductivity) in which the complexes but not the unlinked antibodies adsorb to the hydrophobic resin, thus separating the unlinked proteins (e.g., antibodies) from the complexes adsorbed to the hydrophobic resin; and (ii) eluting the complexes from the hydrophobic resin under conditions in which the complex dissociate from the hydrophobic resin. In some embodiments, the mixture in step (i) further comprises unlinked oligonucleotides. In some embodiments, under the conditions of step (i), the unlinked oligonucleotide also adsorb to the hydrophobic resin, and under the conditions of step (ii), the unlinked oligonucleotides co-elute with the complexes.

In some embodiments, conditions in step (i) that allow for binding of the complexes and optionally the unlinked oligonucleotides, but not the unlinked protein, comprise a conductivity of at least 70 mS/cm (e.g., at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 mS/cm). In some embodiments, conditions in step (i) that allow for binding of the complexes and optionally the unlinked oligonucleotides, but not the unlinked protein, comprise a conductivity of 70-300 mS/cm (e.g., 70-300 mS/cm, 70-200 mS/cm, 70-100 mS/cm, 100-300 mS/cm, 100-200 mS/cm, or 200-300 mS/cm). In some embodiments, conditions in step (i) that allow for binding of the complex and optionally the unlinked oligonucleotides, but not the unlinked protein, comprise a conductivity of 70, 80, 90, 100, 110, 120, 130, 140, 150, 160, 170, 180, 190, 200, 210, 220, 230, 240, 250, 260, 270, 280, 290, or 300 mS/cm. In some embodiments, under conditions that comprises a conductivity of least 70 mS/cm, unlinked antibodies (e.g., unlinked full length IgGs or unlinked Fabs) do not adsorb to the hydrophobic resin (i.e., are in the flow through), thus separating the antibodies from the complexes and optionally the unlinked oligonucleotides adsorbed to the hydrophobic resin. In some embodiments, the method described herein further comprises washing the hydrophobic resin between step (i) and step (ii) with a solution having a conductivity that allows for adsorption of the complexes and optionally the unlinked oligonucleotides, but not the unlinked protein (e.g., unlinked antibodies), to remove the loosely bound but not adsorbed unlinked proteins (e.g., unlinked antibodies).

In some embodiments, the condition is steps (i) and (ii) are achieved using different concentration of an anti-chaotropic salt or molecular agent. In some embodiments, an anti-chaotropic salt or molecular agent is a salt or molecular agent that causes water molecules to favorably interact and stabilizes intramolecular interactions in macro- and bio-molecules, e.g., proteins. Examples of anti-chaotropic salts and molecular agents include sulfates, e.g., ammonium sulfate or sodium sulfate, carbohydrates, e.g., trehalose and glucose, proline, and tert-butanol. In some embodiments, the anti-chaotropic salt used in the methods described herein is ammonium sulfate.

In some embodiments, the mixture in step (i) further comprises at least 500 mM of an anti-chaotropic salt (e.g., ammonium sulfate). For example, the mixture in step (i) may further comprise at least 500 mM, at least 600 mM, at least 700 mM, at least 800 mM, at least 900 mM, at least 1 M, at least 1.1 M, at least 1.2 M, at least 1.3 M, at least 1.4 M, at least 1.5 M, at least 1.6 M, at least 1.7 M, at least 1.8 M, at least 1.9 M, or at least 2 M of an anti-chaotropic salt (e.g., ammonium sulfate). In some embodiments, the mixture in step (i) further comprises 500 mM-1 M of an anti-chaotropic salt (e.g., ammonium sulfate). For example, the mixture in step (i) may further comprises 500 mM-1 M, 500 mM-900 mM, 500 mM-800 mM, 500 mM-700 mM, 500 mM-600 mM, 600 mM-1 M, 600 mM-900 mM, 600 mM-800 mM, 600 mM-700 mM, 700 mM-1 M, 700 mM-900 mM, 700 mM-800 mM, 800 mM-1 M, 800 mM-900 mM, or 900 mM-1 M of an anti-chaotropic salt (e.g., ammonium sulfate). In some embodiments, the mixture in step (i) may further comprises 500 mM, 600 mM, 700 mM, 800 mM, 900 mM, or 1 M of an anti-chaotropic salt (e.g., ammonium sulfate). In some embodiments, the mixture in step (i) further comprises 600 mM, 800 mM, or 1 M of an anti-chaotropic salt (e.g., ammonium sulfate). Under these conditions, unlinked antibodies (e.g., unlinked full length IgG, Fab′, or (Fab′)₂) do not adsorb to the hydrophobic resin (i.e., are in the flow through) while the complexes and optionally the unlinked oligonucleotides adsorb to the hydrophobic resin, thus separating the antibodies from the complexes and optionally the unlinked oligonucleotides.

In some embodiments, the hydrophobic resin is equilibrated prior to step (i) with a solution. In some embodiments, the solution for requilibrating the hydrophobic resin has a conductivity that allows for adsorption of the complexes and optionally the unlinked oligonucleotides (e.g., at least 70 mS/cm), but not the unlinked protein (e.g., unlinked antibodies). In some embodiments, the solution for requilibrating the hydrophobic resin comprises at least 500 mM of an anti-chaotropic salt (e.g., ammonium sulfate). In some embodiments, the solution used for equilibration of the hydrophobic resin comprises at least 500 mM, at least 600 mM, at least 700 mM, at least 800 mM, at least 900 mM, at least 1 M, at least 1.1 M, at least 1.2 M, at least 1.3 M, at least 1.4 M, at least 1.5 M, at least 1.6 M, at least 1.7 M, at least 1.8 M, at least 1.9 M, or at least 2 M of an anti-chaotropic salt (e.g., ammonium sulfate). In some embodiments, the solution used for equilibration of the hydrophobic resin comprises 500 mM-1 M of an anti-chaotropic salt (e.g., ammonium sulfate). For example, the solution used for equilibration of the hydrophobic resin may comprise 500 mM-1 M, 500 mM-900 mM, 500 mM-800 mM, 500 mM-700 mM, 500 mM-600 mM, 600 mM-1 M, 600 mM-900 mM, 600 mM-800 mM, 600 mM-700 mM, 700 mM-1 M, 700 mM-900 mM, 700 mM-800 mM, 800 mM-1 M, 800 mM-900 mM, or 900 mM-1 M of an anti-chaotropic salt (e.g., ammonium sulfate). In some embodiments, the solution used for equilibration of the hydrophobic resin comprises 500 mM, 600 mM, 700 mM, 800 mM, 900 mM, or 1 M of an anti-chaotropic salt (e.g., ammonium sulfate). In some embodiments, the solution used for equilibration of the hydrophobic resin comprises 600 mM, 800 mM, or 1 M of an anti-chaotropic salt (e.g., ammonium sulfate).

In some embodiments, the method described herein further comprising washing the hydrophobic resin between step (i) and step (ii) with a solution having a conductivity that allows for adsorption of the complexes and optionally the unlinked oligonucleotides (e.g., at least 70 mS/cm), but not the unlinked protein (e.g., unlinked antibodies). In some embodiments, the solution used for washing the hydrophobic resin between step (i) and step (ii) comprises at least 500 mM of an anti-chaotropic salt (e.g., ammonium sulfate)) to remove the loosely bound but not adsorbed unlinked proteins (e.g., unlinked antibodies). In some embodiments, the solution used for washing the hydrophobic resin between step (i) and step (ii) comprises at least 500 mM, at least 600 mM, at least 700 mM, at least 800 mM, at least 900 mM, at least 1 M, at least 1.1 M, at least 1.2 M, at least 1.3 M, at least 1.4 M, at least 1.5 M, at least 1.6 M, at least 1.7 M, at least 1.8 M, at least 1.9 M, or at least 2 M of an anti-chaotropic salt (e.g., ammonium sulfate). In some embodiments, the solution used for washing the hydrophobic resin between step (i) and step (ii) comprises 500 mM-1 M of an anti-chaotropic salt (e.g., ammonium sulfate). For example, the solution used for washing the hydrophobic resin between step (i) and step (ii) may comprise 500 mM-1 M, 500 mM-900 mM, 500 mM-800 mM, 500 mM-700 mM, 500 mM-600 mM, 600 mM-1 M, 600 mM-900 mM, 600 mM-800 mM, 600 mM-700 mM, 700 mM-1 M, 700 mM-900 mM, 700 mM-800 mM, 800 mM-1 M, 800 mM-900 mM, or 900 mM-1 M of an anti-chaotropic salt (e.g., ammonium sulfate). In some embodiments, the solution used for washing the hydrophobic resin between step (i) and step (ii) comprises 500 mM, 600 mM, 700 mM, 800 mM, 900 mM, or 1 M of an anti-chaotropic salt (e.g., ammonium sulfate).

In some embodiments, to elute the complexes and optionally the unlinked oligonucleotides from the hydrophobic resin in step (ii), the hydrophobic resin and the adsorbed complexes and optionally unlinked oligonucleotides are subjected to conditions that allow the dissociation of the complexes and optionally the unlinked oligonucleotides from the hydrophobic resin. In some embodiments, conditions in step (ii) that allow dissociation of the complexes and optionally the unlinked oligonucleotides comprises a conductivity of 10-70 mS/cm. For example, conditions in step (ii) that allow dissociation of the complexes and optionally the unlinked oligonucleotides may comprise a conductivity of 10-70 mS/cm, 10-60 mS/cm, 10-50 mS/cm, 10-40 mS/cm, 10-30 mS/cm, 10-20 mS/cm, 20-70 mS/cm, 20-60 mS/cm, 20-50 mS/cm, 20-40 mS/cm, 20-30 mS/cm, 30-70 mS/cm, 30-60 mS/cm, 30-50 mS/cm, 30-40 mS/cm, 40-70 mS/cm, 40-60 mS/cm, 40-50 mS/cm, 50-70 mS/cm, 50-60 mS/cm, or 60-70 mS/cm. In some embodiments, conditions in step (ii) that allow dissociation of the complexes and optionally the unlinked oligonucleotides comprises a conductivity of 10, 15, 20, 25, 30, 35, 40, 45, 50, 55, 60, 65, or 70 mS/cm.

In some embodiments, step (ii) comprises applying an elution solution comprising up to 200 mM (e.g., 10 mM, 25 mM, 50 mM, 100 mM, 150 mM, or 200 mM) of chloride ions and up to 100 mM (e.g., 10 mM, 15 mM, 50 mM, or 100 mM) of an anti-chaotropic salt (e.g., ammonium sulfate) to the hydrophobic resin to elute the complexes. In some embodiments, step (ii) comprises applying an elution solution comprising 0-200 mM (e.g., 0-200 mM, 0-150 mM, 0-100 mM, 0-50 mM, 50-200 mM, 50-150 mM, 50-100 mM, 100-200 mM, 100-150 mM, or 150-200 mM) of chloride ions and 0-100 mM (e.g., 0-100 mM, 0-80 mM, 0-60 mM, 0-40 mM, 0-20 mM, 20-100 mM, 20-80 mM, 20-60 mM, 20-40 mM, 40-100 mM, 40-80 mM, 40-60 mM, 60-100 mM, 60-80 mM, or 80-100 mM) of an anti-chaotropic salt (e.g., ammonium sulfate) to the hydrophobic resin to elute the complexes. In some embodiments, the elution solution used in step (ii) comprises no ammonium sulfate. In some embodiments, the elution solution is Phosphate-buffered saline (PBS, comprising 137 mM NaCl, 2.7 mM KCl, 10 mM Na₂HPO₄, and 1.8 mM KH₂PO₄). In some embodiments, the elution solution comprises up to 25 mM (e.g., 5, 10, 15, 20, or 25 mM) chloride ions and no anti-chaotropic salt (e.g., ammonium sulfate), and optionally further comprises up to 10 mM (e.g., 2, 5, or 10 mM) of phosphate ions. In some embodiments, the elution solution comprises 0-25 mM (e.g., 0-25 mM, 0-20 mM, 0-15 mM, 0-10 mM, 0-5 mM, 5-25 mM, 5-20 mM, 5-15 mM, 5-10 mM, 10-25 mM, 10-20 mM, 10-15 mM, 15-25 mM, 15-20 mM, or 20-25 mM) chloride ions and no anti-chaotropic salt (e.g., ammonium sulfate), and optionally further comprises 0-10 mM (e.g., 0-10 mM, 0-5 mM, or 5-10 mM) of phosphate ions. In some embodiments, the elution solution comprises 25 mM chlorides and no ammonium sulfate, and further comprises 10 mM of phosphate ions.

In some embodiments, the elution solution further comprises counter ions for the phosphate ions and the chloride ions. In some embodiments, the counter ion for phosphate is a calcium, sodium, magnesium, potassium, or manganese. In some embodiments, a source of phosphate ions is NaH₂PO₄, Na₂HPO₄, or Na₃PO₄. In some embodiments, the counter ion for chloride is a calcium, sodium, magnesium, potassium, or manganese. In some embodiments, the counter ion used in the methods described herein is sodium. One of skill in the art would readily understand that many other equivalent salts and ions may be used for the methods described herein.

The elution step may be done using an elution solution comprising a salt (e.g., an anti-chaotropic salt such as ammonium sulfate) having a single/constant concentration, or using an elution solution have a gradient of decreasing salt (e.g., ammonium sulfate) concentration. Using a gradient of decreasing salt (e.g., ammonium sulfate) concentration during elution (step (ii)) allows for separation of complexes with different number of drug:antibody ratio (DAR). For example, as the salt (e.g., ammonium sulfate) concentration decreases in the elution solution, complexes with a lower DAR elute first, then complexes with higher DAR. In some embodiments, complexes having a DAR of 1 (one oligonucleotide conjugated to one antibody) elute at a conductivity of about 35-45 mS/cm (e.g., 35-45 mS/cm, 35-40 mS/cm, or 40-45 mS/cm). In some embodiments, complexes having a DAR of 1 elute at a conductivity of about 35, 36, 37, 38, 39, 40, 41, 42, 43, 44, or 45 mS/cm. In some embodiments, complexes having a DAR of 2 (two oligonucleotide conjugated to one antibody) elute at a conductivity of about 20-35 mS/cm (e.g., 20-35 mS/cm, 20-30 mS/cm, 20-25 mS/cm, 25-35 mS/cm, 25-30 mS/cm, or 30-35 mS/cm). In some embodiments, complexes having a DAR of 2 elute at a conductivity of about 20, 21, 22, 23, 24, 25, 26, 27, 28, 29, 30, 31, 32, 33, 34, or 35 mS/cm. In some embodiments, complexes having a DAR of 3 (three oligonucleotide conjugated to one antibody) elute at a conductivity of about 15-25 mS/cm (e.g., 15-25 mS/cm, 15-20 mS/cm, or 20-25 mS/cm). In some embodiments, complexes having a DAR of 3 elute at a conductivity of about 15, 16, 17, 18, 19, 20, 21, 22, 23, 24, or 25 mS/cm.

In some embodiments, step (ii) comprises applying a gradually decreasing concentration of an anti-chaotropic salt (e.g., ammonium sulfate) to the hydrophobic resin to elute the complexes and optionally the unlinked oligonucleotides. In some embodiments, the concentration of the anti-chaotropic salt (e.g., ammonium sulfate) decreases from at least 500 mM to less than 100 mM. For example, the anti-chaotropic salt (e.g., ammonium sulfate) concentration across the gradient may be 1 M-0 mM, 1 M-50 mM, 1 M-100 mM, 900 mM-0 mM, 900 mM-50 mM, 900 mM-100 mM, 800 mM-0 mM, 800 mM-50 mM, 800 mM-100 mM, 700 mM-0 mM, 700 mM-50 mM, 700 mM-100 mM, 600 mM-0 mM, 600 mM 50 mM, 600 mM-100 mM, 500 mM-0 mM, 500 mM-50 mM, or 500 mM-100 mM. In some embodiments, the anti-chaotropic salt (e.g., ammonium sulfate) concentration across the gradient is 600 mM-100 mM. In some embodiments, the anti-chaotropic salt (e.g., ammonium sulfate) concentration across the gradient is 800 mM-0 mM. In some embodiments, the anti-chaotropic salt (e.g., ammonium sulfate) concentration gradient may be applied over 5-12 (e.g., 5, 6, 7, 8, 9, 10, 11, or 12) column volumes (CVs). In some embodiments, the anti-chaotropic salt (e.g., ammonium sulfate) concentration gradient may be applied over 6-8 column volumes (CVs).

In some embodiments, a wash solution and/or an eluent solution may further comprise a buffering agent in order to maintain a consistent pH. In some embodiments, a wash buffer and/or an eluent buffer comprises a neutral pH. In some embodiments, a wash buffer and/or an eluent buffer comprises a pH of about 6, about 6.5, about 7, about 7.5, about 8, or about 6-8. Examples of buffering agents for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis[2-hydroxyethyl]amino)-2-hydroxypropanesulfonic acid (DIPSO), N-(2-hydroxyethyl)piperazine-N′-(2-hydroxypropanesulfonic acid) (HEPPSO), 4-(2-hydroxyethyl)-1-piperazine propanesulfonic acid (EPPS), N-[tris(hydroxymethyl)-methyl]glycine (Tricine), N,N-bis(2-hydroxyethyl)glycine (Bicine), [(2-hydroxy-1,1-bis(hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris(hydroxymethyl)aminomethane (Tris), and bis[2-hydroxyethyl]iminotris-[hydroxymethyl]methane (Bis-Tris). Other buffers compositions, buffer concentrations, and additional components of a solution for use herein will be apparent to those skilled in the art.

The methods described here may comprise any hydrophobic interaction chromatography (HIC) resin. In some embodiments, a HIC resin comprises butyl, t-butyl, methyl, and/or ethyl functional groups. Typically, a HIC resin comprises hydrophobic functional groups that may interact with biomolecules using hydrophobic interactions.

In some embodiments, a HIC resin comprises one or more hydrophobic functional groups. In some embodiments, a HIC media is a HiTrap Butyl HP resin, CaptoPhenyl resin, Phenyl Sepharose™ 6 resin, Phenyl Sepharose™ High Performance resin, Octyl Sepharose™ High Performance resin, Fractogel™ EMD Propyl resin, Fractogel™ EMD Phenyl resin, Macro-Prep™ Methyl resin, HiScreen Butyl FF, HiScreen Octyl FF, or Tosoh Hexyl.

In some embodiments, a HIC resin may be equilibrated prior to being contacted with a mixture of complex, unlinked oligonucleotide, and unlinked protein. In some embodiments, a HIC resin is equilibrated using a wash solution, as described above. In some embodiments, a HIC resin is equilibrated to bring the pH of the resin to a neutral pH, a pH of 6-8, a pH of about 6.5, a pH of about 7.0, or a pH of about 7.5.

In some embodiments, a HIC resin is packed into a column, e.g., a vertical column. In some embodiments, a column may be used under pressure, optionally pressure from top to bottom or bottom to top. In some embodiments, a column may be used without external pressure, e.g., using gravity flow only. In some embodiments, a HIC resin is used as free resin, e.g., using a batch method. In some embodiments, a batch method may further comprises centrifugation and/or filtration steps following contacting of the resin with the mixture of complex, unlinked oligonucleotide, and unlinked protein.

In some embodiments, the complexes eluted in step (ii) of the hydrophobic chromatography described herein comprises an antibody covalently linked to 1, 2, or 3 oligonucleotides. In some embodiments, the complexes having different numbers of linked oligonucleotides (e.g., 1, 2, or 3) are separated in different elution fractions, e.g., when a decreasing concentration of salt (e.g., ammonium sulfate) is used for elution in step (ii). In some embodiments, the eluent obtained from step (ii) comprises undetectable levels undetectable levels of unlinked antibodies. In some embodiments, the eluent obtained from step (ii) further comprises unlinked oligonucleotides. In some embodiments, the method described herein further comprises isolating the complexes from the unlinked oligonucleotides.

B. Removal of Unlinked Oligonucleotide from Complex Using Mixed-Mode Resin

Purification of complexes comprising protein covalently linked to oligonucleotide, particularly on a large-scale has long been a challenge for scientists in the field. The primary obstacle in purification processes is the substantial removal of unlinked oligonucleotide (e.g., excess oligonucleotide following the conjugation reaction between unlinked protein and unlinked oligonucleotide) from the complex comprising protein covalently linked to oligonucleotide.

It was shown herein that the use of mixed-mode resins that comprise positively-charged metal sites and negatively charged ionic sites (e.g., apatite resin, e.g., hydroxyapatite resin) are effective in purifying complex away from unlinked oligonucleotide. This was a surprising finding in large part because no other purification strategy alternatives were able to remove essentially all unlinked oligonucleotide from compositions comprising protein-oligonucleotide complexes. Further, the mixed-mode resin purification method described herein is advantageous compared to other known methods of removing unlinked oligonucleotides and/or excess salt (desalting). One such known method is size exclusion chromatography (SEC). The mixed-mode resin purification method is advantageous over SEC at least due to its scalability, and higher recovery date. An recovery of at least 90% complexes were achieved using the mixed-mode resin method described herein, while SEC can only achieve 20-30% recovery of the complexes.

In some embodiments, the method of isolating a complex or plurality of complexes each comprising an antibody covalently linked to one or more oligonucleotides described herein comprises: (i) contacting a mixture comprising the complexes and unlinked oligonucleotides with a mixed-mode resin that comprises positively-charged metal sites and negatively charged ionic sites, under conditions in which the complexes adsorb to the mixed-mode resin, and (ii) eluting the complexes from the mixed-mode resin under conditions in which the complexes dissociate from the mixed-mode resin. In some embodiments, wherein the mixture in step (i) was isolated from a hydrophobic interaction chromatography resin prior to step (i). As described herein, in some embodiments, the conditions in step (i) under which the complexes adsorb to the mixed-mode resin may be adjusted to allow or exclude the unlinked oligonucleotides from adsorbing to the mixed-mode resin.

In some embodiments, the conditions in step (i) under which the complexes adsorb to the mixed-mode resin does not allow the unlinked oligonucleotides from adsorbing to the mixed-mode resin, thus separating the complexes from the unlinked oligonucleotides. In some embodiments, the conditions are achieved by including phosphate ions and/or chloride ions in the mixture in step (i) at a concentration that allows the complexes but not the unlinked oligonucleotides to adsorb to the mixed-mode resin. In some embodiments, the mixture comprising the complexes and unlinked oligonucleotides further comprises up to 20 mM phosphate ion and/or up to 30 mM chloride ions. In some embodiments, the mixture comprising the complexes and unlinked oligonucleotides further comprises up to 20 mM (e.g., up to 20 mM, up to 15 mM, up to 10 mM, or up to 5 mM) phosphate ion. Additionally, in some embodiments, the mixture comprising the complexes and unlinked oligonucleotides further comprises up to 30 mM (e.g., up to 30 mM, up to 25 mM, up to 20 mM, up to 15 mM, up to 10 mM, or up to 5 mM) chloride ion. In some embodiments, the mixture comprising the complexes and unlinked oligonucleotides further comprises 5-20 mM (e.g, 5-20 mM, 5-15 mM, 5-10 mM, 10-20 mM, 10-15 mM, or 15-20 mM) phosphate ion and/or 5-30 mM chloride ions (e.g., 5-30 mM, 5-25 mM, 5-20 mM, 5-15 mM, 5-10 mM, 10-30 mM, 10-25 mM, 10-20 mM, 10-15 mM, 15-30 mM, 15-25 mM, 15-20 mM, 20-30 mM, 20-25 mM, or 25-30 mM). In some embodiments, the mixture comprising the complexes and unlinked oligonucleotides further comprises 20 mM, 15 mM, 10 mM, 5 mM, or 1 mM phosphate ion and/or 30 mM, 25 mM, 20 mM, 15 mM, 10 mM, or 5 mM chloride ion. In some embodiments, the mixture comprising the complexes and unlinked oligonucleotides further comprises 20 mM phosphate ion and/or 30 mM chloride ion, e.g. 20 mM phosphate ion and 30 mM chloride ion. In some embodiments, the mixture comprising the complexes and unlinked oligonucleotides further comprises up to 10 mM phosphate ions and/or up to 25 mM chloride ions. In some embodiments, the mixture comprising the complexes and unlinked oligonucleotides further comprises 5-10 mM phosphate ions and/or 5-25 mM chloride ions. In some embodiments, the mixture comprising the complexes and unlinked oligonucleotides further comprises 10 mM phosphate ions and/or 25 mM chloride ions, e.g., 10 mM phosphate ions and 25 mM chloride ions. Under these conditions, the unlinked oligonucleotides remain in the flow through and do not adsorb to the mixed-mode resin. In some embodiments, the mixed-mode resin may further be washed between step (i) and step (ii) under these same conditions to remove unlinked oligonucleotides that are loosely bound but not adsorbed to the mixed-mode resin.

In some embodiments, the conditions in step (i) under which the complexes adsorb to the mixed-mode resin also allow some or all (e.g., at least 10%, at least 20%, at least 30%, at least 40%, at least 50%, at least 60%, at least 70%, at least 80%, at least 90%, or 100%) of the unlinked oligonucleotides to adsorb to the mixed-mode resin, In some embodiments, the conditions are achieved by including phosphate ions and/or chloride ions in the mixture in step (i) at a concentration that allows both the complexes and the unlinked oligonucleotides to adsorb to the mixed-mode resin. In some embodiments, the mixture comprising the complexes and unlinked oligonucleotides further comprises up to 5 mM phosphate ion and/or up to 10 mM chloride ions. In some embodiments, the mixture comprising the complexes and unlinked oligonucleotides further comprises up to 5 mM (e.g., up to 5 mM, up to 4 mM, up to 3 mM, up to 2 mM, or up to 1 mM) phosphate ion. Additionally, in some embodiments, the mixture comprising the complexes and unlinked oligonucleotides further comprises up to 10 mM (e.g., up to 10 mM, up to 9 mM, up to 8 mM, up to 7 mM, up to 6 mM, up to 5 mM, up to 4 mM, up to 3 mM, up to 2 mM, or up to 1 mM) chloride ion. In some embodiments, the mixture comprising the complexes and unlinked oligonucleotides further comprises 1-5 mM (e.g., 1-5 mM, 1-4 mM, 1-3 mM, 1-2 mM, 2-5 mM, 2-4 mM, 2-3 mM, 3-5 mM, 3-4 mM, or 4-5 mM) phosphate ion, and/or 1-10 mM (e.g., 1-10 mM, 1-8 mM, 1-6 mM, 1-4 mM, 1-2 mM, 2-10 mM, 2-8 mM, 2-6 mM, 2-4 mM, 4-10 mM, 4-8 mM, 4-6 mM, 6-10 mM, 6-8 mM, or 8-10 mM) chloride ion. In some embodiments, the mixture comprising the complexes and unlinked oligonucleotides further comprises 5 mM, 4 mM, 3 mM, 2 mM, or 1 mM phosphate ion and/or 10 mM, 9 mM, 8 mM, 7 mM, 6 mM, 5 mM, 4 mM, 3 mM, 2 mM, or 1 mM chloride ion. In some embodiments, the mixture comprising the complexes and unlinked oligonucleotides further comprises up to 3 mM phosphate ions and/or up to 8 mM chloride ions. In some embodiments, the mixture comprising the complexes and unlinked oligonucleotides further comprises 1-3 mM (e.g., 1, 2, or 3 mM) phosphate ions and/or 1-8 mM (e.g., 1, 2, 3, 4, 5, 6, 7, or 8 mM) chloride ions. In some embodiments, the mixture comprising the complexes and unlinked oligonucleotides further comprises 3 mM phosphate ions and/or 8 mM chloride ions, e.g., 3 mM phosphate ions and 8 mM chloride ions. Under these conditions, some of all of the unlinked oligonucleotides also adsorb to the mixed-mode resin.

In some embodiments, when some or all of the unlinked oligonucleotides also adsorb to the mix-mode resin, the methods described herein further comprises washing the mixed-mode resin between step (i) and step (ii) with a solution that would dissociate the unlinked oligonucleotides but not the complexes from the mixed-mode resin. In some embodiments, the solution used for washing comprises up to 20 mM phosphate ions and/or up to 30 mM chloride ions, e.g. 20 mM phosphate ion and 30 mM chloride ion. In some embodiments, the solution used for washing comprises up to 20 mM (e.g., up to 20 mM, up to 15 mM, up to 10 mM, or up to 5 mM) phosphate ion. Additionally, in some embodiments, the solution used for washing comprises up to 30 mM (e.g., up to 30 mM, up to 25 mM, up to 20 mM, up to 15 mM, up to 10 mM, or up to 5 mM) chloride ion. In some embodiments, the solution used for washing comprises 5-20 mM (e.g, 5-20 mM, 5-15 mM, 5-10 mM, 10-20 mM, 10-15 mM, or 15-20 mM) phosphate ion and/or 5-30 mM chloride ions (e.g., 5-30 mM, 5-25 mM, 5-20 mM, 5-15 mM, 5-10 mM, 10-30 mM, 10-25 mM, 10-20 mM, 10-15 mM, 15-30 mM, 15-25 mM, 15-20 mM, 20-30 mM, 20-25 mM, or 25-30 mM). In some embodiments, the solution used for washing comprises 20 mM, 15 mM, 10 mM, 5 mM, or 1 mM phosphate ion and/or 30 mM, 25 mM, 20 mM, 15 mM, 10 mM, or 5 mM chloride ion. In some embodiments, the solution used for washing comprises 20 mM phosphate ion and/or 30 mM chloride ion, e.g., 20 mM phosphate ion and 30 mM chloride ion. In some embodiments, the solution used for washing comprises up to 10 mM phosphate ions and/or up to 25 mM chloride ions, e.g., 10 mM phosphate ions and up to 25 mM chloride ions.

In some embodiments, to elute the complexes from the mixed-mode resin, in step (ii), the mixed-mode and the bound complexes are subject to conditions that allow the dissociation of the complexes from the mixed-mode resin. In some embodiments, conditions in step (ii) that allow dissociation of the complexes are achieved by applying to the mixed-mode resin an elution solution comprising a higher concentration of phosphate ions and/or chloride ions. The elution step may be done using an elution solution comprising a single phosphate ion concentration, or using an elution solution have a gradient of increasing phosphate ion concentration. Using a gradient of increasing phosphate ion concentration during elution (step (ii)) allows for separation of complexes with different number of drug:antibody ratio (DAR). For example, as the increasing phosphate ion concentration increases in the elution solution, complexes with a lower DAR elutes first, then complexes with higher DAR.

In some embodiments, step (ii) comprises applying an elution solution comprising at least 30 mM phosphate ions and/or at least 50 mM chloride ions to the mixed-mode resin to elute the complexes. In some embodiments, the elution solution comprises at least 30 mM (e.g., at least 30 mM, at least 40 mM, at least 50 mM, at least 60 mM, at least 70 mM, at least 80 mM, at least 90 mM, at least 100 mM, at least 110 mM, at least 120 mM, at least 130 mM, at least 140 mM, or at least 150 mM) phosphate ions. Additionally, in some embodiments, the elution solution comprises at least 50 mM (e.g., at least 50 mM, at least 60 mM, at least 70 mM, at least 80 mM, at least 90 mM, at least 100 mM, at least 110 mM, at least 120 mM, at least 130 mM, at least 140 mM, at least 150 mM, at least 160 mM, at least 170 mM, at least 180 mM, at least 190 mM, or at least 200 mM) chloride ions. In some embodiments, the elution solution comprises at least 100 mM phosphate ions and/or at least 100 mM chloride ions. In some embodiments, the elution solution comprises 100 mM phosphate ions and 100 mM chloride ions.

In some embodiments, the mixtures and solutions used in the method described herein further comprises counter ions for the phosphate ion and/or the chloride ions. In some embodiments, the counter ion for phosphate is a calcium, sodium, magnesium, potassium, or manganese. In some embodiments, the counter ion used in the methods described herein is sodium. In some embodiments, a source of phosphate ions is NaH₂PO₄, Na₂HPO₄, or Na₃PO₄. In some embodiments, the counter ion for chloride is a calcium, sodium, magnesium, potassium, or manganese. In some embodiments, a source of chloride ions is NaCl. One of skill in the art would readily understand that many other equivalent salts and ions may be used for the methods described herein.

In some embodiments, a wash solution and/or an eluent solution may further comprise a buffering agent in order to maintain a consistent pH. In some embodiments, a wash buffer and/or an eluent buffer comprises a neutral pH. In some embodiments, a wash buffer and/or an eluent buffer comprises a pH of about 6, about 6.5, about 7, about 7.5, about 8, or about 6-8. Examples of buffering agents for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis[2-hydroxyethyl]amino)-2-hydroxypropanesulfonic acid (DIPSO), N-(2-hydroxyethyl)piperazine-N′-(2-hydroxypropanesulfonic acid) (HEPPSO), 4-(2-hydroxyethyl)-1-piperazine propanesulfonic acid (EPPS), N-[tris(hydroxymethyl)-methyl]glycine (Tricine), N,N-bis(2-hydroxyethyl)glycine (Bicine), [(2-hydroxy-1,1-bis(hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris(hydroxymethyl)aminomethane (Tris), and bis[2-hydroxyethyl]iminotris-[hydroxymethyl]methane (Bis-Tris). Other buffers compositions, buffer concentrations, and additional components of a solution for use herein will be apparent to those skilled in the art.

Any mixed-mode resin that comprises positively-charged metal sites and negatively charged ionic sites may be used in accordance with the present disclosure. In some embodiments, the mixed-mode resin used in the methods described herein is an apatite resin. In some embodiments, the apatite resin is a hydroxyapatite resin, a ceramic hydroxyapatite resin, a hydroxyfluoroapatite resin, a fluoroapatite resin, or a chlorapatite resin. An apatite resin may comprise apatite in any form and is typically used as a chromatographic solid phase in the separation and purification of biomolecules, e.g., complexes described herein, using affinity, ion exchange, hydrophobic interactions, or combinations thereof.

In some embodiments, a hydroxyapatite resin is a Bio-Gel HT resin, e.g., from Bio-Rad Laboratories, Inc. (Hercules, Calif., USA). In some embodiments, a ceramic hydroxyapatite resin is a Bio-Scale Mini CHT resin, e.g., from Bio-Rad Laboratories, Inc. In some embodiments, an apatite resin, e.g., ceramic hydroxyapatite, comprises spherical particles of apatite. In some embodiments, the spherical particles of apatite are about 10 microns to about 100 microns, about 25 microns to about 50 microns, about 20 microns, about 30 microns, about 40 microns, about 50 microns, about 60 microns, or about 80 microns in diameter. In some embodiments, an apatite resin, e.g., ceramic hydroxyapatite, is Type I (medium porosity and a high binding capacity) or Type II (larger porosity and a lower binding capacity). In some embodiments, an apatite particle may be used in admixture with another separation medium or support.

In some embodiments, a mixed-mode resin may be equilibrated prior to being contacted with a mixture of complex and unlinked oligonucleotide. In some embodiments, a mixed-mode resin is equilibrated using a wash solution, as described above. In some embodiments, a mixed-mode resin is equilibrated to bring the pH of the resin to a neutral pH, a pH of 6-8, a pH of about 6.5, a pH of about 7.0, or a pH of about 7.5.

In some embodiments, a mixed-mode resin is packed into a column, e.g., a vertical column. In some embodiments, a column may be used under pressure, optionally pressure from top to bottom or bottom to top. In some embodiments, a column may be used without external pressure, e.g., using gravity flow only. In some embodiments, a mixed-mode resin is used as free resin, e.g., using a batch method. In some embodiments, a batch method may further comprises centrifugation and/or filtration steps following contacting of the resin with the mixture of complex and unlinked oligonucleotide.

In some embodiments, the complexes eluted in step (ii) of the mixed-mode resin chromatography described herein comprises an antibody covalently linked to 1, 2, or 3 oligonucleotides. In some embodiments, the complexes having different numbers of linked oligonucleotides (e.g., 1, 2, or 3) are separated in different elution fractions. In some embodiments, the eluent obtained from step (ii) comprises undetectable levels of unlinked oligonucleotide.

C. Removal of Unlinked Proteins (e.g., Antibodies) and Unlinked Oligonucleotide from Complex Using Hydrophobic Resin Followed by Mixed-Mode Resin

In some aspects, the present disclosure provide methods of isolating a complex or plurality of complexes each comprising an antibody covalently linked to one or more oligonucleotides, the method comprising: (i) contacting a first mixture comprising the complexes, unlinked antibodies, and unlinked oligonucleotides with a hydrophobic resin under conditions in which the complexes and the unlinked oligonucleotides but not the unlinked antibodies adsorb to the hydrophobic resin, thus separating the unlinked antibodies from the complexes and the unlinked oligonucleotides adsorbed to the hydrophobic resin; and (ii) obtaining a second mixture comprising the complexes and the unlinked oligonucleotides by eluting the complexes and the unlinked oligonucleotides from the hydrophobic resin under conditions in which the complexes dissociate from the hydrophogic resin; (iii) contacting the second mixture obtained in step (ii) with a mixed-mode resin that comprises positively-charged metal sites and negatively charged ionic sites, under conditions in which the complexes adsorb to the mixed-mode resin, and (iv) eluting the complexes from the mixed-mode resin under conditions in which the complexes dissociate from the mixed-mode resin.

In some embodiments, the hydrophobic resin comprises a hydrophobic moiety selected from butyl, t-butyl, phenyl, ether, amide, or propyl groups. In some embodiments, the mixed-mode resin is an apatite resin, optionally wherein the apatite resin is a hydroxyapatite resin, a ceramic hydroxyapatite resin, a hydroxyfluoroapatite resin, a fluoroapatite resin, or a chlorapatite resin. In some embodiments, the conditions in step (i) comprise a conductivity of at least 70 mS/cm (e.g., 70-300 mS/cm, 70-200 mS/cm, 70-100 mS/cm, 100-300 mS/cm, 100-200 mS/cm, or 200-300 mS/cm), and the conditions in step (ii) comprises a conductivity of 10-70 mS/cm (e.g., 10-70 mS/cm, 10-60 mS/cm, 10-50 mS/cm, 10-40 mS/cm, 10-30 mS/cm, 10-20 mS/cm, 20-70 mS/cm, 20-60 mS/cm, 20-50 mS/cm, 20-40 mS/cm, 20-30 mS/cm, 30-70 mS/cm, 30-60 mS/cm, 30-50 mS/cm, 30-40 mS/cm, 40-70 mS/cm, 40-60 mS/cm, 40-50 mS/cm, 50-70 mS/cm, 50-60 mS/cm, or 60-70 mS/cm).

In some embodiments, the conditions in step (i) and or step (ii) are achieved using an anti-chaotropic salt, e.g., ammonium sulfate. In some embodiments, the hydrophobic resin is equilibrated prior to step (i), e.g., equilibrated with a solution comprising at least 500 mM (e.g., at least 500 mM, at least 600 mM, at least 700 mM, at least 800 mM, at least 900 mM, at least 1 M, at least 1.1 M, at least 1.2 M, at least 1.3 M, at least 1.4 M, at least 1.5 M, at least 1.6 M, at least 1.7 M, at least 1.8 M, at least 1.9 M, or at least 2 M) of ammonium sulfate.

In some embodiments, the mixture in step (i) further comprises at least 500 mM (e.g., at least 500 mM, at least 600 mM, at least 700 mM, at least 800 mM, at least 900 mM, at least 1 M, at least 1.1 M, at least 1.2 M, at least 1.3 M, at least 1.4 M, at least 1.5 M, at least 1.6 M, at least 1.7 M, at least 1.8 M, at least 1.9 M, or at least 2 M) of ammonium sulfate. In some embodiments, the mixture in step (i) further comprises 500 mM-1 M (500 mM-1 M, 500 mM-900 mM, 500 mM-800 mM, 500 mM-700 mM, 500 mM-600 mM, 600 mM-1 M, 600 mM-900 mM, 600 mM-800 mM, 600 mM-700 mM, 700 mM-1 M, 700 mM-900 mM, 700 mM-800 mM, 800 mM-1 M, 800 mM-900 mM, or 900 mM-1 M) of ammonium sulfate.

In some embodiments, the method further comprises washing the hydrophobic resin between step (i) and step (ii) with a solution comprising at least 500 mM (e.g., at least 500 mM, at least 600 mM, at least 700 mM, at least 800 mM, at least 900 mM, at least 1 M, at least 1.1 M, at least 1.2 M, at least 1.3 M, at least 1.4 M, at least 1.5 M, at least 1.6 M, at least 1.7 M, at least 1.8 M, at least 1.9 M, or at least 2 M) of ammonium sulfate.

In some embodiments, step (ii) comprises applying a first elution solution comprising up to 200 mM (e.g., 0-200 mM, 0-150 mM, 0-100 mM, 0-50 mM, 50-200 mM, 50-150 mM, 50-100 mM, 100-200 mM, 100-150 mM, or 150-200 mM) of chloride ions and up to 100 mM (e.g., 0-100 mM, 0-80 mM, 0-60 mM, 0-40 mM, 0-20 mM, 20-100 mM, 20-80 mM, 20-60 mM, 20-40 mM, 40-100 mM, 40-80 mM, 40-60 mM, 60-100 mM, 60-80 mM, or 80-100 mM) of ammonium sulfate to the hydrophobic resin to elute the complexes and the unlinked oligonucleotides.

In some embodiments, the first elution solution does not contain ammonium sulfate. In some embodiments, the first elution solution is PBS. In some embodiments, the first elution solution comprises up to 25 mM chloride ions (e.g., 0-25 mM, 0-20 mM, 0-15 mM, 0-10 mM, 0-5 mM, 5-25 mM, 5-20 mM, 5-15 mM, 5-10 mM, 10-25 mM, 10-20 mM, 10-15 mM, 15-25 mM, 15-20 mM, or 20-25 mM) and no ammonium sulfate.

In some embodiments, step (ii) comprises applying a gradually decreasing concentration of ammonium sulfate to the hydrophobic resin to elute the complexes and the unlinked oligonucleotides, optionally wherein the concentration of ammonium sulfate decreases from at least 500 mM to less than 100 mM and/or the gradually decreasing concentration of ammonium sulfate is applied over 5-12 column volumes (CVs), optionally 6-8 CVs. For example, the ammonium sulfate concentration across the gradient may be 1 M-0 mM, 1 M-50 mM, 1 M-100 mM, 900 mM-0 mM, 900 mM-50 mM, 900 mM-100 mM, 800 mM-0 mM, 800 mM-50 mM, 800 mM-100 mM, 700 mM-0 mM, 700 mM-50 mM, 700 mM-100 mM, 600 mM-0 mM, 600 mM-50 mM, 600 mM-100 mM, 500 mM-0 mM, 500 mM 50 mM, or 500 mM-100 mM. In some embodiments, ammonium sulfate concentration across the gradient is 600 mM-100 mM. In some embodiments, ammonium sulfate concentration across the gradient is 800 mM-0 mM. In some embodiments, the anti-chaotropic salt (e.g., ammonium sulfate) concentration gradient may be applied over 5-12 (e.g., 5, 6, 7, 8, 9, 10, 11, or 12) column volumes (CVs). In some embodiments, the anti-chaotropic salt (e.g., ammonium sulfate) concentration gradient may be applied over 6-8 column volumes (CVs).

In some embodiments, the second mixture in step (iii) further comprises up to 20 mM (e.g, 5-20 mM, 5-15 mM, 5-10 mM, 10-20 mM, 10-15 mM, or 15-20 mM) phosphate ions and up to 30 mM (e.g., 5-30 mM, 5-25 mM, 5-20 mM, 5-15 mM, 5-10 mM, 10-30 mM, 10-25 mM, 10-20 mM, 10-15 mM, 15-30 mM, 15-25 mM, 15-20 mM, 20-30 mM, 20-25 mM, or 25-30 mM) chloride ions, In some embodiments, the second mixture in step (iii) further comprises up to 10 mM (e.g., 5-10 mM) phosphate ions and up to 25 mM (e.g., 5-25 mM) chloride ions. In some embodiments, the second mixture in step (iii) further comprises 20 mM phosphate ions and 30 mM chloride ions, In some embodiments, the second mixture in step (iii) further comprises 10 mM phosphate ions and 25 mM chloride ions. Under these conditions the unlinked oligonucleotide does not adsorb to the mixed-mode resin in step (iii).

In some embodiments, the second mixture in step (iii) further comprises up to 5 mM (e.g., 1-5 mM) phosphate ions and/or up to 10 mM (e.g., 5-10 mM) chloride ions, optionally wherein the second mixture in step (iii) further comprises up to 3 mM (e.g., 1-3 mM) phosphate ions and/or up to 8 mM (e.g., 5-8 mM) chloride ions. In some embodiments, the second mixture in step (iii) further comprises up to 5 mM phosphate ions and 10 mM chloride ions. In some embodiments, the second mixture in step (iii) further comprises up to 3 mM phosphate ions and 8 mM chloride ions. Under these conditions, some or all of the unlinked oligonucleotide adsorb to the mixed-mode resin in step (iii).

In some embodiments, when some or all of the unlinked oligonucleotides also adsorb to the mix-mode resin, the method further comprising washing the mixed-mode resin between step (iii) and step (iv) with a solution comprising up to 20 mM (e.g, 5-20 mM, 5-15 mM, 5-10 mM, 10-20 mM, 10-15 mM, or 15-20 mM) phosphate ions and up to 30 mM (e.g., 5-30 mM, 5-25 mM, 5-20 mM, 5-15 mM, 5-10 mM, 10-30 mM, 10-25 mM, 10-20 mM, 10-15 mM, 15-30 mM, 15-25 mM, 15-20 mM, 20-30 mM, 20-25 mM, or 25-30 mM) chloride ions to remove the unlinked oligonucleotide from the mixed mode resin. In some embodiments, the solution for washing comprises up to 10 mM (e.g., 5-10 mM) phosphate ions and up to 25 mM (e.g., 5-25 mM) chloride ions.

In some embodiments, step (iv) comprises applying a second elution solution comprising at least 30 mM (e.g., at least 30 mM, at least 40 mM, at least 50 mM, at least 60 mM, at least 70 mM, at least 80 mM, at least 90 mM, at least 100 mM, at least 110 mM, at least 120 mM, at least 130 mM, at least 140 mM, or at least 150 mM) phosphate ions and at least 50 mM (e.g., at least 50 mM, at least 60 mM, at least 70 mM, at least 80 mM, at least 90 mM, at least 100 mM, at least 110 mM, at least 120 mM, at least 130 mM, at least 140 mM, at least 150 mM, at least 160 mM, at least 170 mM, at least 180 mM, at least 190 mM, or at least 200 mM) chloride ions to the mixed-mode resin to elute the complexes. In some embodiments, wherein the second elution solution comprises 100 mM phosphate ions and 100 mM chloride.

In some embodiments, the mixtures and solutions used in the method described herein further comprises counter ions for the phosphate ion and/or the chloride ions. In some embodiments, the counter ion for phosphate is a calcium, sodium, magnesium, potassium, or manganese. In some embodiments, the counter ion used in the methods described herein is sodium. In some embodiments, a source of phosphate ions is NaH₂PO₄, Na₂HPO₄, or Na₃PO₄. In some embodiments, the counter ion for chloride is a calcium, sodium, magnesium, potassium, or manganese. In some embodiments, a source of chloride ions is NaCl. One of skill in the art would readily understand that many other equivalent salts and ions may be used for the methods described herein.

In some embodiments, a wash solution and/or an eluent solution may further comprise a buffering agent in order to maintain a consistent pH. In some embodiments, a wash buffer and/or an eluent buffer comprises a neutral pH. In some embodiments, a wash buffer and/or an eluent buffer comprises a pH of about 6, about 6.5, about 7, about 7.5, about 8, or about 6-8. Examples of buffering agents for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis[2-hydroxyethyl]amino)-2-hydroxypropanesulfonic acid (DIPSO), N-(2-hydroxyethyl)piperazine-N′-(2-hydroxypropanesulfonic acid) (HEPPSO), 4-(2-hydroxyethyl)-1-piperazine propanesulfonic acid (EPPS), N-[tris(hydroxymethyl)-methyl]glycine (Tricine), N,N-bis(2-hydroxyethyl)glycine (Bicine), [(2-hydroxy-1,1-bis(hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris(hydroxymethyl)aminomethane (Tris), and bis[2-hydroxyethyl]iminotris-[hydroxymethyl]methane (Bis-Tris). Other buffers compositions, buffer concentrations, and additional components of a solution for use herein will be apparent to those skilled in the art.

D. Compositions of Purified Complexes

The methods described herein may produce substantially purified complexes, wherein a composition of purified complexes do not comprise detectable quantities of unlinked oligonucleotide or unlinked protein. In some embodiments, a composition of purified complexes comprises a molar or weight ratio of complex:unlinked oligonucleotide that is at least 9:1, at least 95:5, 96:4, 97:3, 98:2, 99:1, 99.5:5, or higher. In some embodiments, a composition of purified complex comprises a molar or weight ratio of complex:unlinked protein that is at least 9:1, at least 95:5, 96:4, 97:3, 98:2, 99:1, 99.5:5, or higher.

In some embodiments, a composition of purified complexes does not comprise detectable levels (e.g., detectable quantities) of unlinked protein. In some embodiments, a composition of purified complexes does not comprise detectable levels (e.g., detectable quantities) of unlinked oligonucleotide.

In some embodiments, a composition of purified complexes comprises less than 10%, less than 9%, less than 8%, less than 7%, less than 6%, less than 5%, less than 4%, less than 3%, less than 2%, less than 5%, or less than 0.5% of unlinked protein (e.g., antibody) by molar ratio. In some embodiments, a composition of purified complexes comprises less than 10%, less than 9%, less than 8%, less than 7%, less than 6%, less than 5%, less than 4%, less than 3%, less than 2%, less than 5%, or less than 0.5% of unlinked oligonucleotides by molar ratio. In some embodiments, a composition of purified complexes comprises less than 10%, less than 9%, less than 8%, less than 7%, less than 6%, less than 5%, less than 4%, less than 3%, less than 2%, less than 5%, or less than 0.5% of unlinked protein (e.g., antibody) by molar ratio, and comprises less than 10%, less than 9%, less than 8%, less than 7%, less than 6%, less than 5%, less than 4%, less than 3%, less than 2%, less than 5%, or less than 0.5% of unlinked oligonucleotides by molar ratio.

In some embodiments, a composition of purified complexes comprises at least 90% (e.g., at least 90%, at least 91%, at least 92%, at least 93%, at least 94%, at least 95%, at least 96%, at least 97%, at least 98%, at least 99%, at least 99.5%, or at least 99.9%) complexes (i.e., protein (e.g., antibody) linked to one or more oligonucleotides) by molar ratio.

In some embodiments, a composition of purified complexes comprises protein (e.g., antibody) linked to one oligonucleotide, two oligonucleotides, three oligonucleotides and/or more oligonucleotides. In some embodiments, in a composition of purified complexes, at least 50% (e.g., at least 60%, at least 70%, at least 80%, at least 90%, at least 95%) or more of the complexes comprise a protein (e.g., antibody) linked to one oligonucleotide (DAR1). In some embodiments, in a composition of purified complexes, about 50%, 60%, 70%, 80%, 90%, or 95% complexes comprise a protein (e.g., antibody) linked to one oligonucleotide (DAR1).

In some embodiments, in a composition of purified complexes, about 5%, 10%, 15%, 20%, 25%, 30%, or more of complexes comprise a protein (e.g., antibody) linked to two oligonucleotides (DAR2). In some embodiments, in a composition of purified complexes, about 1%, 2%, 3%, 5%, 7%, 10%, 20%, or more of complexes comprise a protein (e.g., antibody) linked to three or more oligonucleotides (DAR3+).

### III. Complexes

Provided herein are complexes that comprise a targeting agent, e.g. an antibody, covalently linked to a molecular payload, e.g., an oligonucleotide. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens. A complex may be used to modulate the activity or function of at least one gene, protein, and/or nucleic acid. In some embodiments, the molecular payload present with a complex is responsible for the modulation of a gene, protein, and/or nucleic acids. A molecular payload may be a small molecule, protein, nucleic acid, oligonucleotide, or any molecular entity capable of modulating the activity or function of a gene, protein, and/or nucleic acid in a cell. In some embodiments, a molecular payload is an oligonucleotide that targets a muscle disease allele in muscle cells.

In some embodiments, a complex comprises a muscle-targeting agent, e.g. an anti-transferrin receptor antibody, covalently linked to a molecular payload, e.g. an antisense oligonucleotide that targets a muscle disease allele.

In some embodiments, a complex is useful for treating a muscle disease, in which a molecular payload affects the activity of the corresponding gene provided in Table 1. For example, depending on the condition, a molecular payload may modulate (e.g., decrease, increase) transcription or expression of the gene, modulate the expression of a protein encoded by the gene, or to modulate the activity of the encoded protein. In some embodiments, the molecular payload is an oligonucleotide that comprises a strand having a region of complementarity to a target gene provided in Table 1.

TABLE 1
List of muscle diseases and corresponding genes.

| | Gene | |
|---|---|---|
| Disease | Symbol | GenBank Accession No. |
| Adult Pompe | GAA | NM_000152; NM_01079803; |
| | | NM_001079804 |
| Adult Pompe | GYS1 | NM_001161587; NM_002103 |
| Centronuclear myopathy (CNM) | DNM2 | NM_001190716; NM_004945; |
| | | NM_001005362; |
| | | NM_001005360; |
| | | NM_001005361; NM_007871 |
| Duchenne muscular dystrophy | DMD | NM_004023; NM_04020; |
| | | NM_004018; NM_04012 |
| Facioscapulohumeral muscular | DUX4 | NM_001306068; |
| dystrophy (FSHD) | | NM_001363820; |
| | | NM_001205218; NM_001293798 |
| Familial hypertrophic | MYBPC3 | NM_000256 |
| cardiomyopathy | | |
| Familial hypertrophic | MYH6 | NM_002471; NM_01164171; |
| cardiomyopathy | | NM_010856 |
| Familial hypertrophic | MYH7 | NM_000257; NM_80728 |
| cardiomyopathy | | |
| Familial hypertrophic | TNNI3 | NM_000363 |
| cardiomyopathy | | |
| Familial hypertrophic | TNNT2 | NM_001001432; |
| cardiomyopathy | | NM_001001431; NM_000364; |
| | | NM_01001430; |
| | | NM_01276347; |
| | | NM_01276346; NM_01276345 |
| Fibrodysplasia Ossificans | ACVR1 | NM_01105; NM_01347663; |
| Progressiva (FOP) | | NM_01347664; |
| | | NM_01347665; |
| | | NM_01347666; |
| | | NM_01347667; NM_01111067 |
| Friedreich’s ataxia (FRDA) | FXN | NM_01161706; NM_181425; |
| | | NM_00144 |
| Inclusion body myopathy 2 | GNE | NM_01190383; |
| | | NM_01190384; |
| | | NM_01128227; NM_05476; |
| | | NM_01190388 |
| Laing distal myopathy | MYH7 | NM_00257; NM_80728 |
| Myofibrillar myopathy | BAG3 | NM_04281 |
| Myofibrillar myopathy | CRYAB | NM_01885; NM_01330379; |
| | | NM_01289807; NM_01289808 |
| Myofibrillar myopathy | DES | NM_01927 |
| Myofibrillar myopathy | DNAJB6 | NM_05494; NM_58246 |
| Myofibrillar myopathy | FHL1 | NM_01159701; |
| | | NM_01159699; |
| | | NM_01159702; |
| | | NM_01159703; |
| | | NM_01159704; NM_01159700; |
| | | NM_01167819; |
| | | NM_01330659; NM_01449; |
| | | NM_01077362 |
| Myofibrillar myopathy | FLNC | NM_01458; NM_01127487 |
| Myofibrillar myopathy | LDB3 | NM_07078; NM_01171611; |
| | | NM_01171610; |
| | | NM_01080114; |
| | | NM_01080115; NM_01080116 |
| Myofibrillar myopathy | MYOT | NM_01300911; NM_06790; |
| | | NM_01135940 |
| Myofibrillar myopathy | PLEC | NM_201378; NM_201379; |
| | | NM_201380; NM_201381; |
| | | NM_201382; NM_201383; |
| | | NM_201384; NM_00445 |
| Myofibrillar myopathy | TTN | NM_133432; NM_133379; |
| | | NM_133437; NM_03319; |
| | | NM_01256850; |
| | | NM_01267550; NM_133378 |
| Myotonia congenita (autosomal | CLCN1 | NM_00083; NM_13491 |
| dominant form, Thomsen Disease) | | |
| Myotonic dystrophy type I | DMPK | NM_01081563; NM_04409; |
| | | NM_01081560; |
| | | NM_01081562; |
| | | NM_01288764; |
| | | NM_01288765; NM_01288766 |
| Myotonic dystrophy type II | CNBP | NM_001127192; |
| | | NM_001127193; |
| | | NM_001127194; |
| | | NM_001127195; |
| | | NM_001127196; NM_003418 |
| Myotubular myopathy | MTM1 | NM_000252 |
| Oculopharyngeal muscular dystrophy | PABPN1 | NM_004643 |
| Paramyotonia congenita | SCN4A | NM_000334 |

A. Cell-Targeting Agents

Some aspects of the disclosure provide cell-targeting agents, e.g., muscle-targeting proteins, e.g., for delivering a oligonucleotide to a muscle cell. In some embodiments, such cell-targeting proteins are capable of binding to a specific cell, e.g., via specifically binding to an antigen on said cell, and delivering an associated oligonucleotide to the cell. In some embodiments, the oligonucleotide is bound (e.g., covalently bound) to the cell-targeting agent and is internalized into said cell upon binding of the cell-targeting agent to an antigen on the cell, e.g., via endocytosis.

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload (e.g., oligonucleotide) into muscle tissue. Binding to muscle surface recognition elements followed by endocytosis can allow even large molecules such as antibodies to enter muscle cells. As another example oligonucleotides conjugated to transferrin or anti-transferrin receptor antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

Muscle cells encompassed by the present disclosure include, but are not limited to, skeletal muscle cells, smooth muscle cells, cardiac muscle cells, myoblasts and myocytes.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 March, 39(13):78309; the entire contents of each of which are incorporated herein by reference.

a. Anti-Transferrin Receptor Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload (e.g., oligonucleotide), into a muscle cell. As used herein, an antibody that binds to a transferrin receptor may be referred to as an anti-transferrin receptor antibody. Antibodies that bind, e.g. specifically bind, to a transferrin receptor may be internalized into the cell, e.g. through receptor-mediated endocytosis, upon binding to a transferrin receptor.

It should be appreciated that anti-transferrin receptor antibodies may be produced, synthesized, and/or derivatized using several known methodologies, e.g. library design using phage display. Exemplary methodologies have been characterized in the art and are incorporated by reference (Diez, P. et al. “High-throughput phage-display screening in array format”, Enzyme and microbial technology, 2015, 79, 34-41; Christoph M. H. and Stanley, J. R. “Antibody Phage Display: Technique and Applications” J Invest Dermatol. 2014, 134:2; Engleman, Edgar (Ed.) “Human Hybridomas and Monoclonal Antibodies.” 1985, Springer.). In other embodiments, an anti-transferrin antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052).

Any appropriate anti-transferrin receptor antibodies may be used in the complexes disclosed herein. Examples of anti-transferrin receptor antibodies, including associated references and binding epitopes, are listed in Table 2. In some embodiments, the anti-transferrin receptor antibody comprises the complementarity determining regions (CDR-H1, CDR-H2, CDR-H3, CDR-L1, CDR-L2, and CDR-L3) of any of the anti-transferrin receptor antibodies provided herein, e.g., anti-transferrin receptor antibodies listed in Table 2.

TABLE 2
List of anti-transferrin receptor antibody clones, including
associated references and binding epitope information.

| Antibody | | |
|---|---|---|
| Clone Name | Reference(s) | Epitope/Notes |
| OKT9 | U.S. Pat. No. 4,364,934, filed Dec. 4, 1979, | Apical domain of TfR |
| | entitled “MONOCLONAL ANTIBODY | (residues 305-366 of |
| | TO A HUMAN EARLY THYMOCYTE | human TfR sequence |
| | ANTIGEN AND METHODS FOR | XM_052730.3, |
| | PREPARING SAME” | available in GenBank) |
| | Schneider C. et al. “Structural features of | |
| | the cell surface receptor for transferrin that | |
| | is recognized by the monoclonal antibody | |
| | OKT9.” J Biol Chem. 1982, | |
| | 257:14, 8516-8522. | |
| (From JCR) | WO 2015/098989, filed | Apical domain |
| Clone M11 | Dec. 24, 2014, “Novel anti-Transferrin | (residues 230-244 and |
| Clone M23 | receptor antibody that passes through | 326-347 of TfR) and |
| Clone M27 | blood-brain barrier” | protease-like domain |
| Clone B84 | U.S. Pat. No. 9,994,641, filed | (residues 461-473) |
| | Feb. 24, 2014, “Novel anti-Transferrin | |
| | receptor antibody that passes through | |
| | blood-brain barrier” | |
| (From | WO 2016/081643, filed May 26, 2016, | Apical domain and |
| Genentech) | entitled “ANTI-TRANSFERRIN | non-apical regions |
| 7A4, 8A2, | RECEPTOR ANTIBODIES AND | |
| 15D2, 10D11, | METHODS OF USE” | |
| 7B10, 15G11, | U.S. Pat. No. 9,708,406, filed | |
| 16G5, 13C3, | May 20, 2014, “Anti-transferrin receptor | |
| 16G4, 16F6, | antibodies and methods of use” | |
| 7G7, 4C2, | | |
| 1B12, and | | |
| 13D4 | | |
| (From | Lee et al. “Targeting Rat Anti- | |
| Armagen) | Mouse Transferrin Receptor Monoclonal | |
| 8D3 | Antibodies through Blood-Brain Barrier in | |
| | Mouse” 2000, J Pharmacol. Exp. Ther., | |
| | 292:1048-1052. | |
| | U.S. patent application 2010/077498, filed | |
| | Sept. 11, 2008, entitled “COMPOSITIONS | |
| | AND METHODS FOR BLOOD-BRAIN | |
| | BARRIER DELIVERY IN THE MOUSE” | |
| OX26 | Haobam, B. et al. 2014. Rab17- | |
| | mediated recycling endosomes contribute | |
| | to autophagosome formation in response to | |
| | Group A Streptococcus invasion. Cellular | |
| | microbiology. 16:1806-21. | |
| DF1513 | Ortiz-Zapater E et al. Trafficking | |
| | of the human transferrin receptor in plant | |
| | cells: effects of tyrphostin A23 and | |
| | brefeldin A. Plant J 48:757-70 (2006). | |
| 1A1B2, | Commercially available anti- | Novus Biologicals |
| 66IG10, | transferrin receptor antibodies. | 8100 Southpark Way, |
| MEM-189, | | A-8 Littleton CO |
| JF0956, 29806, | | 80120 |
| 1A1B2, | | |
| TFRC/1818, | | |
| 1E6, 66Ig10, | | |
| TFRC/1059, | | |
| Q1/71, 23D10, | | |
| 13E4, | | |
| TFRC/1149, | | |
| ER-MP21, | | |
| YTA74.4, | | |
| BU54, 2B6, | | |
| RI7 217 | | |
| (From | U.S. patent application 2011/0311544A1, | Does not compete |
| INSERM) | filed Jun. 15, 2005, entitled “ANTI-CD71 | with OKT9 |
| BA120g | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 epitope” |
| | Jun. 7, 2004, entitled “TRANSFERRIN | |
| | RECEPTOR ANTIBODIES” | |
| (Salk Institute) | Trowbridge, I.S. et al. “Anti-transferrin | |
| B3/25 | receptor monoclonal antibody and | |
| T58/30 | toxin-antibody conjugates affect | |
| | growth of human tumour cells.” | |
| | Nature, 1981, volume 294, | |
| | pages 171-173 | |
| R17 217.1.3, | Commercially available anti- | BioXcell |
| 5E9C11, | transferrin receptor antibodies. | 10 Technology Dr., |
| OKT9 | | Suite 2B |
| (BE0023 | | West Lebanon, NH |
| clone) | | 03784-1671 USA |
| BK19.9, | Gatter, K.C. et al. “Transferrin | |
| B3/25, T56/14 | receptors in human tissues: their | |
| and T58/1 | distribution and possible clinical | |
| | relevance.” J Clin Pathol. 1983 | |
| | May;36(5):539-45. | |

In some embodiments, the muscle-targeting agent is an anti-transferrin receptor antibody. In some embodiment, an anti-transferrin receptor antibody specifically binds to a transferrin protein having an amino acid sequence as disclosed herein. In some embodiments, an anti-transferrin receptor antibody may specifically bind to any extracellular epitope of a transferrin receptor or an epitope that becomes exposed to an antibody, including the apical domain, the transferrin binding domain, and the protease-like domain. In some embodiments, an anti-transferrin receptor antibody binds to an amino acid segment of a human or non-human primate transferrin receptor, as provided in SEQ ID Nos. 1-3 in the range of amino acids C89 to F760. In some embodiments, an anti-transferrin receptor antibody specifically binds with binding affinity of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. Anti-transferrin receptor antibodies used herein may be capable of competing for binding with other anti-transferrin receptor antibodies, e.g. OKT9, 8D3, that bind to transferrin receptor with 10⁻³ M, 10⁻⁴ M, 10⁻⁵ M, 10⁻⁶ M, 10⁻⁷ M, or less.

An example human transferrin receptor amino acid sequence, corresponding to NCBI sequence NP_003225.2 (transferrin receptor protein 1 isoform 1, Homo sapiens) is as follows:

(SEQ ID NO: 1)
MMDQARSAFSNLFGGEPLSYTRFSLARQVDGDNSH
VEMKLAVDEEENADNNTKANVTKPKRCSGSICYGT
IAVIVFFLIGFMIGYLGYCKGVEPKTECERLAGTE
SPVREEPGEDFPAARRLYWDDLKRKLSEKLDSTDF
TGTIKLLNENSYVPREAGSQKDENLALYVENQFRE
FKLSKVWRDQHFVKIQVKDSAQNSVIIVDKNGRLV
YLVENPGGYVAYSKAATVTGKLVHANFGTKKDFED
LYTPVNGSIVIVRAGKITFAEKVANAESLNAIGVL
IYMDQTKFPIVNAELSFFGHAHLGTGDPYTPGFPS
FNHTQFPPSRSSGLPNIPVQTISRAAAEKLFGNME
GDCPSDWKTDSTCRMVTSESKNVKLTVSNVLKEIK
ILNIFGVIKGFVEPDHYVVVGAQRDAWGPGAAKSG
VGTALLLKLAQMFSDMVLKDGFQPSRSIIFASWSA
GDFGSVGATEWLEGYLSSLHLKAFTYINLDKAVLG
TSNFKVSASPLLYTLIEKTMQNVKHPVTGQFLYQD
SNWASKVEKLTLDNAAFPFLAYSGIPAVSFCFCED
TDYPYLGTTMDTYKELIERIPELNKVARAAAEVAG
QFVIKLTHDVELNLDYERYNSQLLSFVRDLNQYRA
DIKEMGLSLQWLYSARGDFFRATSRLTTDFGNAEK
TDRFVMKKLNDRVMRVEYHFLSPYVSPKESPFRHV
FWGSGSHTLPALLENLKLRKQNNGAFNETLFRNQL
ALATWTIQGAANALSGDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 2)
MMDQARSAFSNLFGGEPLSYTRFSLARQVDGDNSH
VEMKLGVDEEENTDNNTKPNGTKPKRCGGNICYGT
IAVIIFFLIGFMIGYLGYCKGVEPKTECERLAGTE
SPAREEPEEDFPAAPRLYWDDLKRKLSEKLDTTDF
TSTIKLLNENLYVPREAGSQKDENLALYIENQFRE
FKLSKVWRDQHFVKIQVKDSAQNSVIIVDKNGGLV
YLVENPGGYVAYSKAATVTGKLVHANFGTKKDFED
LDSPVNGSIVIVRAGKITFAEKVANAESLNAIGVL
IYMDQTKFPIVKADLSFFGHAHLGTGDPYTPGFPS
FNHTQFPPSQSSGLPNIPVQTISRAAAEKLFGNME
GDCPSDWKTDSTCKMVTSENKSVKLTVSNVLKETK
ILNIFGVIKGFVEPDHYVVVGAQRDAWGPGAAKSS
VGTALLLKLAQMFSDMVLKDGFQPSRSIIFASWSA
GDFGSVGATEWLEGYLSSLHLKAFTYINLDKAVLG
TSNFKVSASPLLYTLIEKTMQDVKHPVTGRSLYQD
SNWASKVEKLTLDNAAFPFLAYSGIPAVSFCFCED
TDYPYLGTTMDTYKELVERIPELNKVARAAAEVAG
QFVIKLTHDTELNLDYERYNSQLLLFLRDLNQYRA
DVKEMGLSLQWLYSARGDFFRATSRLTTDFRNAEK
RDKFVMKKLNDRVMRVEYYFLSPYVSPKESPFRHV
FWGSGSHTLSALLESLKLRRQNNSAFNETLFRNQL
ALATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 3)
MMDQARSAFSNLFGGEPLSYTRFSLARQVDGDNSH
VEMKLGVDEEENTDNNTKANGTKPKRCGGNICYGT
IAVIIFFLIGFMIGYLGYCKGVEPKTECERLAGTE
SPAREEPEEDFPAAPRLYWDDLKRKLSEKLDTTDF
TSTIKLLNENLYVPREAGSQKDENLALYIENQFRE
FKLSKVWRDQHFVKIQVKDSAQNSVIIVDKNGGLV
YLVENPGGYVAYSKAATVTGKLVHANFGTKKDFED
LDSPVNGSIVIVRAGKITFAEKVANAESLNAIGVL
IYMDQTKFPIVKADLSFFGHAHLGTGDPYTPGFPS
FNHTQFPPSQSSGLPNIPVQTISRAAAEKLFGNME
GDCPSDWKTDSTCKMVTSENKSVKLTVSNVLKETK
ILNIFGVIKGFVEPDHYVVVGAQRDAWGPGAAKSS
VGTALLLKLAQMFSDMVLKDGFQPSRSIIFASWSA
GDFGSVGATEWLEGYLSSLHLKAFTYINLDKAVLG
TSNFKVSASPLLYTLIEKTMQDVKHPVTGRSLYQD
SNWASKVEKLTLDNAAFPFLAYSGIPAVSFCFCED
TDYPYLGTTMDTYKELVERIPELNKVARAAAEVAG
QFVIKLTHDTELNLDYERYNSQLLLFLRDLNQYRA
DVKEMGLSLQWLYSARGDFFRATSRLTTDFRNAEK
RDKFVMKKLNDRVMRVEYYFLSPYVSPKESPFRHV
FWGSGSHTLSALLESLKLRRQNNSAFNETLFRNQL
ALATWTIQGAANALSGDVWDIDNEF.

An example mouse transferrin receptor amino acid sequence, corresponding to NCBI sequence NP_001344227.1 (transferrin receptor protein 1, Mus musculus) is as follows:

(SEQ ID NO: 4)
MMDQARSAFSNLFGGEPLSYTRFSLARQVDGDNSH
VEMKLAADEEENADNNMKASVRKPKRFNGRLCFAA
IALVIFFLIGFMSGYLGYCKRVEQKEECVKLAETE
ETDKSETMETEDVPTSSRLYWADLKTLLSEKLNSI
EFADTIKQLSQNTYTPREAGSQKDESLAYYIENQF
HEFKFSKVWRDEHYVKIQVKSSIGQNMVTIVQSNG
NLDPVESPEGYVAFSKPTEVSGKLVHANFGTKKDF
EELSYSVNGSLVIVRAGEITFAEKVANAQSFNAIG
VLIYMDKNKFPVVEADLALFGHAHLGTGDPYTPGF
PSFNHTQFPPSQSSGLPNIPVQTISRAAAEKLFGK
MEGSCPARWNIDSSCKLELSQNQNVKLIVKNVLKE
RRILNIFGVIKGYEEPDRYVVVGAQRDALGAGVAA
KSSVGTGLLLKLAQVFSDMISKDGFRPSRSIIFAS
WTAGDFGAVGATEWLEGYLSSLHLKAFTYINLDKV
VLGTSNFKVSASPLLYTLMGKIMQDVKHPVDGKSL
YRDSNWISKVEKLSFDNAAYPFLAYSGIPAVSFCF
CEDADYPYLGTRLDTYEALTQKVPQLNQMVRTAAE
VAGQLIIKLTHDVELNLDYEMYNSKLLSFMKDLNQ
FKTDIRDMGLSLQWLYSARGDYFRATSRLTTDFHN
AEKTNRFVMREINDRIMKVEYHFLSPYVSPRESPF
RHIFWGSGSHTLSALVENLKLRQKNITAFNETLFR
NQLALATWTIQGVANALSGDIWNIDNEF

In some embodiments, an anti-transferrin receptor antibody binds to an amino acid segment of the receptor as follows:

(SEQ ID NO: 5)
FVKIQVKDSAQNSVIIVDKNGRLVYLVENPGGYVA
YSKAATVTGKLVHANFGTKKDFEDLYTPVNGSIVI
VRAGKITFAEKVANAESLNAIGVLIYMDQTKFPIV
NAELSFFGHAHLGTGDPYTPGFPSFNHTQFPPSRS
SGLPNIPVQTISRAAAEKLFGNMEGDCPSDWKTDS
TCRMVTSESKNVKLTVSNVLKE

and does not inhibit the binding interactions between transferrin receptors and transferrin and/or human hemochromatosis protein (also known as HFE).

Appropriate methodologies may be used to obtain and/or produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g. ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988).

In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

Some aspects of the disclosure provide proteins that bind to transferrin receptor (e.g., an extracellular portion of the transferrin receptor). In some embodiments, transferrin receptor antibodies provided herein bind specifically to transferrin receptor (e.g., human transferrin receptor). Transferrin receptors are internalizing cell surface receptors that transport transferrin across the cellular membrane and participate in the regulation and homeostasis of intracellular iron levels. In some embodiments, transferrin receptor antibodies provided herein bind specifically to transferrin receptor from human, non-human primates, mouse, rat, etc. In some embodiments, transferrin receptor antibodies provided herein bind to human transferrin receptor. In some embodiments, transferrin receptor antibodies provided herein specifically bind to human transferrin receptor. In some embodiments, transferrin receptor antibodies provided herein bind to an apical domain of human transferrin receptor. In some embodiments, transferrin receptor antibodies provided herein specifically bind to an apical domain of human transferrin receptor.

In some embodiments, transferrin receptor antibodies of the present disclosure include one or more of the CDR-H (e.g., CDR-H1, CDR-H2, and CDR-H3) amino acid sequences from any one of the anti-transferrin receptor antibodies selected from Table 2. In some embodiments, transferrin receptor antibodies include the CDR-H1, CDR-H2, and CDR-H3 as provided for any one of the anti-transferrin receptor antibodies selected from Table 2. In some embodiments, anti-transferrin receptor antibodies include the CDR-L1, CDR-L2, and CDR-L3 as provided for any one of the anti-transferrin receptor antibodies selected from Table 2. In some embodiments, anti-transferrin antibodies include the CDR-H1, CDR-H2, CDR-H3, CDR-L1, CDR-L2, and CDR-L3 as provided for any one of the anti-transferrin receptor antibodies selected from Table 2. The disclosure also includes any nucleic acid sequence that encodes a molecule comprising a CDR-H1, CDR-H2, CDR-H3, CDR-L1, CDR-L2, or CDR-L3 as provided for any one of the anti-transferrin receptor antibodies selected from Table 2. In some embodiments, antibody heavy and light chain CDR3 domains may play a particularly important role in the binding specificity/affinity of an antibody for an antigen. Accordingly, anti-transferrin receptor antibodies of the disclosure may include at least the heavy and/or light chain CDR3s of any one of the anti-transferrin receptor antibodies selected from Table 2.

In some examples, any of the anti-transferrin receptor antibodies of the disclosure have one or more CDR (e.g., CDR-H or CDR-L) sequences substantially similar to any of the CDR-H1, CDR-H2, CDR-H3, CDR-L1, CDR-L2, and/or CDR-L3 sequences from one of the anti-transferrin receptor antibodies selected from Table 2. In some embodiments, the position of one or more CDRs along the VH (e.g., CDR-H1, CDR-H2, or CDR-H3) and/or VL (e.g., CDR-L1, CDR-L2, or CDR-L3) region of an antibody described herein can vary by one, two, three, four, five, or six amino acid positions so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDR-H1, CDR-H2, or CDR-H3) and/or VL (e.g., CDR-L1, CDR-L2, or CDR-L3) region of an antibody described herein can vary (e.g., be shorter or longer) by one, two, three, four, five, or more amino acids, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived).

Accordingly, in some embodiments, a CDR-L1, CDR-L2, CDR-L3, CDR-H1, CDR-H2, and/or CDR-H3 described herein may be one, two, three, four, five or more amino acids shorter than one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, a CDR-L1, CDR-L2, CDR-L3, CDR-H1, CDR-H2, and/or CDR-H3 described herein may be one, two, three, four, five or more amino acids longer than one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or CDR-H3 described herein can be extended by one, two, three, four, five or more amino acids compared to one or more of the CDRs described herein (e.g., CDRS from any of the anti-transferrin receptor antibodies selected from Table 2) so long as immunospecific binding to transferrin receptor (e.g., human transferrin receptor is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or CDR-H3 described herein can be extended by one, two, three, four, five or more amino acids compared to one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or CDR-H3 described herein can be shortened by one, two, three, four, five or more amino acids compared to one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or CDR-H3 described herein can be shortened by one, two, three, four, five or more amino acids compared to one or more of the CDRs described herein (e.g., CDRS from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to transferrin receptor (e.g., human transferrin receptor) is maintained, for example, using binding assays and conditions described in the art.

In some examples, any of the anti-transferrin receptor antibodies of the disclosure have one or more CDR (e.g., CDR-H or CDR-L) sequences substantially similar to any one of the anti-transferrin receptor antibodies selected from Table 2. For example, the antibodies may include one or more CDR sequence(s) from any of the anti-transferrin receptor antibodies selected from Table 2 containing up to 5, 4, 3, 2, or 1 amino acid residue variations as compared to the corresponding CDR region in any one of the CDRs provided herein (e.g., CDRs from any of the anti-transferrin receptor antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a transferrin receptor protein (e.g., a human transferrin receptor protein), for example, as determined based on a crystal structure. Some aspects of the disclosure provide transferrin receptor antibodies that comprise one or more of the heavy chain variable (VH) and/or light chain variable (VL) domains provided herein. In some embodiments, any of the VH domains provided herein include one or more of the CDR-H sequences (e.g., CDR-H1, CDR-H2, and CDR-H3) provided herein, for example, any of the CDR-H sequences provided in any one of the anti-transferrin receptor antibodies selected from Table 2. In some embodiments, any of the VL domains provided herein include one or more of the CDR-L sequences (e.g., CDR-L1, CDR-L2, and CDR-L3) provided herein, for example, any of the CDR-L sequences provided in any one of the anti-transferrin receptor antibodies selected from Table 2.

In some embodiments, anti-transferrin receptor antibodies of the disclosure include any antibody that includes a heavy chain variable domain and/or a light chain variable domain of any anti-transferrin receptor antibody, such as any one of the anti-transferrin receptor antibodies selected from Table 2. In some embodiments, anti-transferrin receptor antibodies of the disclosure include any antibody that includes the heavy chain variable and light chain variable pairs of any anti-transferrin receptor antibody, such as any one of the anti-transferrin receptor antibodies selected from Table 2.

Aspects of the disclosure provide anti-transferrin receptor antibodies having a heavy chain variable (VH) and/or a light chain variable (VL) domain amino acid sequence homologous to any of those described herein. In some embodiments, the anti-transferrin receptor antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-transferrin receptor antibody, such as any one of the anti-transferrin receptor antibodies selected from Table 2. In some embodiments, the homologous heavy chain variable and/or a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein. In some embodiments, any of the anti-transferrin receptor antibodies provided herein comprise a heavy chain variable sequence and a light chain variable sequence that comprises a framework sequence that is at least 75%, 80%, 85%, 90%, 95%, 98%, or 99% identical to the framework sequence of any anti-transferrin receptor antibody, such as any one of the anti-transferrin receptor antibodies selected from Table 2.

In some embodiments, an anti-transferrin receptor antibody, which specifically binds to transferrin receptor (e.g., human transferrin receptor), comprises a light chain variable VL domain comprising any of the CDR-L domains (CDR-L1, CDR-L2, and CDR-L3), or CDR-L domain variants provided herein, of any of the anti-transferrin receptor antibodies selected from Table 2. In some embodiments, an anti-transferrin receptor antibody, which specifically binds to transferrin receptor (e.g., human transferrin receptor), comprises a light chain variable VL domain comprising the CDR-L1, the CDR-L2, and the CDR-L3 of any anti-transferrin receptor antibody, such as any one of the anti-transferrin receptor antibodies selected from Table 2. In some embodiments, the anti-transferrin receptor antibody comprises a light chain variable (VL) region sequence comprising one, two, three or four of the framework regions of the light chain variable region sequence of any anti-transferrin receptor antibody, such as any one of the anti-transferrin receptor antibodies selected from Table 2. In some embodiments, the anti-transferrin receptor antibody comprises one, two, three or four of the framework regions of a light chain variable region sequence which is at least 75%, 80%, 85%, 90%, 95%, or 100% identical to one, two, three or four of the framework regions of the light chain variable region sequence of any anti-transferrin receptor antibody, such as any one of the anti-transferrin receptor antibodies selected from Table 2. In some embodiments, the light chain variable framework region that is derived from said amino acid sequence consists of said amino acid sequence but for the presence of up to 10 amino acid substitutions, deletions, and/or insertions, preferably up to 10 amino acid substitutions. In some embodiments, the light chain variable framework region that is derived from said amino acid sequence consists of said amino acid sequence with 1, 2, 3, 4, 5, 6, 7, 8, 9 or 10 amino acid residues being substituted for an amino acid found in an analogous position in a corresponding non-human, primate, or human light chain variable framework region.

In some embodiments, an anti-transferrin receptor antibody that specifically binds to transferrin receptor comprises the CDR-L1, the CDR-L2, and the CDR-L3 of any anti-transferrin receptor antibody, such as any one of the anti-transferrin receptor antibodies selected from Table 2. In some embodiments, the antibody further comprises one, two, three or all four VL framework regions derived from the VL of a human or primate antibody. The primate or human light chain framework region of the antibody selected for use with the light chain CDR sequences described herein, can have, for example, at least 70% (e.g., at least 75%, 80%, 85%, 90%, 95%, 98%, or at least 99%) identity with a light chain framework region of a non-human parent antibody. The primate or human antibody selected can have the same or substantially the same number of amino acids in its light chain complementarity determining regions to that of the light chain complementarity determining regions of any of the antibodies provided herein, e.g., any of the anti-transferrin receptor antibodies selected from Table 2. In some embodiments, the primate or human light chain framework region amino acid residues are from a natural primate or human antibody light chain framework region having at least 75% identity, at least 80% identity, at least 85% identity, at least 90% identity, at least 95% identity, at least 98% identity, at least 99% (or more) identity with the light chain framework regions of any anti-transferrin receptor antibody, such as any one of the anti-transferrin receptor antibodies selected from Table 2. In some embodiments, an anti-transferrin receptor antibody further comprises one, two, three or all four VL framework regions derived from a human light chain variable kappa subfamily. In some embodiments, an anti-transferrin receptor antibody further comprises one, two, three or all four VL framework regions derived from a human light chain variable lambda subfamily.

In some embodiments, any of the anti-transferrin receptor antibodies provided herein comprise a light chain variable domain that further comprises a light chain constant region. In some embodiments, the light chain constant region is a kappa, or a lambda light chain constant region. In some embodiments, the kappa or lambda light chain constant region is from a mammal, e.g., from a human, monkey, rat, or mouse. In some embodiments, the light chain constant region is a human kappa light chain constant region. In some embodiments, the light chain constant region is a human lambda light chain constant region. It should be appreciated that any of the light chain constant regions provided herein may be variants of any of the light chain constant regions provided herein. In some embodiments, the light chain constant region comprises an amino acid sequence that is at least 75%, 80%, 85%, 90%, 95%, 98%, or 99% identical to any of the light chain constant regions of any anti-transferrin receptor antibody, such as any one of the anti-transferrin receptor antibodies selected from Table 2.

In some embodiments, the anti-transferrin receptor antibody is any anti-transferrin receptor antibody, such as any one of the anti-transferrin receptor antibodies selected from Table 2.

In some embodiments, an anti-transferrin receptor antibody comprises a VL domain comprising the amino acid sequence of any anti-transferrin receptor antibody, such as any one of the anti-transferrin receptor antibodies selected from Table 2, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In some embodiments, an anti-transferrin receptor antibody comprises any of the VL domains, or VL domain variants, and any of the VH domains, or VH domain variants, wherein the VL and VH domains, or variants thereof, are from the same antibody clone, and wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, an antibody of the disclosure can bind to a target antigen (e.g., transferrin receptor) with relatively high affinity, e.g., with a K_{D} less than 10⁻⁶ M, 10⁻⁷ M, 10⁻⁸ M, 10⁻⁹ M, 10⁻¹⁰ M, 10⁻¹¹ M or lower. For example, anti-transferrin receptor antibodies can bind to a transferrin receptor protein (e.g., human transferrin receptor) with an affinity between 5 pM and 500 nM, e.g., between 50 pM and 100 nM, e.g., between 500 pM and 50 nM. The disclosure also includes antibodies that compete with any of the antibodies described herein for binding to a transferrin receptor protein (e.g., human transferrin receptor) and that have an affinity of 50 nM or lower (e.g., 20 nM or lower, 10 nM or lower, 500 pM or lower, 50 pM or lower, or 5 pM or lower). The affinity and binding kinetics of the anti-transferrin receptor antibody can be tested using any suitable method including but not limited to biosensor technology (e.g., OCTET or BIACORE).

In some embodiments, an antibody of the disclosure can bind to a target antigen (e.g., transferrin receptor) with relatively high affinity, e.g., with a K_{D} less than 10⁻⁶ M, 10⁻⁷ M, 10⁻⁸ M, 10⁻⁹ M, 10⁻¹⁰ M, 10⁻¹¹ M or lower. For example, anti-transferrin receptor antibodies can bind to a transferrin receptor protein (e.g., human transferrin receptor) with an affinity between 5 pM and 500 nM, e.g., between 50 pM and 100 nM, e.g., between 500 pM and 50 nM. The disclosure also includes antibodies that compete with any of the antibodies described herein for binding to a transferrin receptor protein (e.g., human transferrin receptor) and that have an affinity of 50 nM or lower (e.g., 20 nM or lower, 10 nM or lower, 500 pM or lower, 50 pM or lower, or 5 pM or lower). The affinity and binding kinetics of the anti-transferrin receptor antibody can be tested using any suitable method including but not limited to biosensor technology (e.g., OCTET or BIACORE).

In some embodiments, the muscle-targeting agent is a transferrin receptor antibody (e.g., the antibody and variants thereof as described in International Application Publication WO 2016/081643, incorporated herein by reference).

The heavy chain and light chain CDRs of the antibody according to different definition systems are provided in Table 3. The different definition systems, e.g., the Kabat definition, the Chothia definition, and/or the contact definition have been described. See, e.g., (e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

TABLE 3
Heavy chain and light chain CDRs of a
mouse transferrin receptor antibody

| | | | |
|---|---|---|---|
| CDRs | Kabat | Chothia | Contact |
| CDR-H1 | SYWMH | GYTFTSY | TSYWMH |
| | (SEQ ID | (SEQ ID | (SEQ ID |
| | NO: 17) | NO: 23) | NO: 25) |
| CDR-H2 | EINPTNGRT | NPTNGR | WIGEINP |
| | NYIEKFKS | | TNGRTN |
| | (SEQ ID | (SEQ ID | (SEQ ID |
| | NO: 18) | NO: 24) | NO: 26) |
| CDR-H3 | GTRAYHY | GTRAYHY | ARGTRA |
| | (SEQ ID | (SEQ ID | (SEQ ID |
| | NO: 19) | NO: 19) | NO: 27) |
| CDR-L1 | RASDNLY | RASDNLY | YSNLAWY |
| | SNLA | SNLA | |
| | (SEQ ID | (SEQ ID | (SEQ ID |
| | NO: 20) | NO: 20) | NO: 28) |
| CDR-L2 | DATNLAD | DATNLAD | LLVYDAT |
| | | | NLA |
| | (SEQ ID | (SEQ ID | (SEQ ID |
| | NO: 21) | NO: 21) | NO: 29) |
| CDR-L3 | QHFWGTPLT | QHFWGTPLT | QHFWGTPL |
| | (SEQ ID | (SEQ ID | (SEQ ID |
| | NO: 22) | NO: 22) | NO: 30) |

The heavy chain variable domain (VH) and light chain variable domain sequences are also provided:

VH
(SEQ ID NO: 33)
QVQLQQPGAELVKPGASVKLSCKASGYTFTSYWMH
WVKQRPGQGLEWIGEINPTNGRTNYIEKFKSKATL
TVDKSSSTAYMQLSSLTSEDSAVYYCARGTRAYHY
WGQGTSVTVSS
VL
(SEQ ID NO: 34)
DIQMTQSPASLSVSVGETVTITCRASDNLYSNLAW
YQQKQGKSPQLLVYDATNLADGVPSRFSGSGSGTQ
YSLKINSLQSEDFGTYYCQHFWGTPLTFGAGTKLE
LK

In some embodiments, the transferrin receptor antibody of the present disclosure comprises a CDR-H1, a CDR-H2, and a CDR-H3 that are the same as the CDR-H1, CDR-H2, and CDR-H3 shown in Table 3. Alternatively or in addition, the transferrin receptor antibody of the present disclosure comprises a CDR-L1, a CDR-L2, and a CDR-L3 that are the same as the CDR-L1, CDR-L2, and CDR-L3 shown in Table 3.

In some embodiments, the transferrin receptor antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1, CDR-H2, and CDR-H3 as shown in Table 3. “Collectively” means that the total number of amino acid variations in all of the three heavy chain CDRs is within the defined range. Alternatively or in addition, the transferrin receptor antibody of the present disclosure may comprise a CDR-L1, a CDR-L2, and a CDR-L3, which collectively contains no more than 5 amino acid variations (e.g., no more than 5, 4, 3, 2 or 1 amino acid variation) as compared with the CDR-L1, CDR-L2, and CDR-L3 as shown in Table 3.

In some embodiments, the transferrin receptor antibody of the present disclosure comprises a CDR-H1, a CDR-H2, and a CDR-H3, at least one of which contains no more than 3 amino acid variations (e.g., no more than 3, 2, or 1 amino acid variation) as compared with the counterpart heavy chain CDR as shown in Table 3. Alternatively or in addition, the transferrin receptor antibody of the present disclosure may comprise CDR-L1, a CDR-L2, and a CDR-L3, at least one of which contains no more than 3 amino acid variations (e.g., no more than 3, 2, or 1 amino acid variation) as compared with the counterpart light chain CDR as shown in Table 3.

In some embodiments, the transferrin receptor antibody of the present disclosure comprises a CDR-L3, which contains no more than 3 amino acid variations (e.g., no more than 3, 2, or 1 amino acid variation) as compared with the CDR-L3 as shown in Table 3. In some embodiments, the transferrin receptor antibody of the present disclosure comprises a CDR-L3 containing one amino acid variation as compared with the CDR-L3 as shown in Table 3. In some embodiments, the transferrin receptor antibody of the present disclosure comprises a CDR-L3 of QHFAGTPLT (SEQ ID NO: 31 according to the Kabat and Chothia definition system) or QHFAGTPL (SEQ ID NO: 32 according to the Contact definition system). In some embodiments, the transferrin receptor antibody of the present disclosure comprises a CDR-H1, a CDR-H2, a CDR-H3, a CDR-L1 and a CDR-L2 that are the same as the CDR-H1, CDR-H2, and CDR-H3 shown in Table 3, and comprises a CDR-L3 of QHFAGTPLT (SEQ ID NO: 31 according to the Kabat and Chothia definition system) or QHFAGTPL (SEQ ID NO: 32 according to the Contact definition system).

In some embodiments, the transferrin receptor antibody of the present disclosure comprises heavy chain CDRs that collectively are at least 80% (e.g., 80%, 85%, 90%, 95%, or 98%) identical to the heavy chain CDRs as shown in Table 3. Alternatively or in addition, the transferrin receptor antibody of the present disclosure comprises light chain CDRs that collectively are at least 80% (e.g., 80%, 85%, 90%, 95%, or 98%) identical to the light chain CDRs as shown in Table 3.

In some embodiments, the transferrin receptor antibody of the present disclosure comprises a VH comprising the amino acid sequence of SEQ ID NO: 33. Alternatively or in addition, the transferrin receptor antibody of the present disclosure comprises a VL comprising the amino acid sequence of SEQ ID NO: 34.

In some embodiments, the transferrin receptor antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 33. Alternatively or in addition, the transferrin receptor antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 34.

In some embodiments, the transferrin receptor antibody of the present disclosure comprises a VH comprising an amino acid sequence that is at least 80% (e.g., 80%, 85%, 90%, 95%, or 98%) identical to the VH as set forth in SEQ ID NO: 33. Alternatively or in addition, the transferrin receptor antibody of the present disclosure comprises a VL comprising an amino acid sequence that is at least 80% (e.g., 80%, 85%, 90%, 95%, or 98%) identical to the VL as set forth in SEQ ID NO: 34.

In some embodiments, the transferrin receptor antibody of the present disclosure is a humanized antibody (e.g., a humanized variant of an antibody). In some embodiments, the transferrin receptor antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 shown in Table 3, and comprises a humanized heavy chain variable region and/or a humanized light chain variable region.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Table 3) into the IGKV1-NL1*01 and IGHV1-3*01 human variable domains. In some embodiments, the transferrin receptor antibody of the present disclosure is a humanized variant comprising one or more amino acid substitutions at positions 9, 13, 17, 18, 40, 45, and 70 as compared with the VL as set forth in SEQ ID NO: 34, and/or one or more amino acid substitutions at positions 1, 5, 7, 11, 12, 20, 38, 40, 44, 66, 75, 81, 83, 87, and 108 as compared with the VH as set forth in SEQ ID NO: 33. In some embodiments, the transferrin receptor antibody of the present disclosure is a humanized variant comprising amino acid substitutions at all of positions 9, 13, 17, 18, 40, 45, and 70 as compared with the VL as set forth in SEQ ID NO: 34, and/or amino acid substitutions at all of positions 1, 5, 7, 11, 12, 20, 38, 40, 44, 66, 75, 81, 83, 87, and 108 as compared with the VH as set forth in SEQ ID NO: 33.

In some embodiments, the transferrin receptor antibody of the present disclosure is a humanized antibody and contains the residues at positions 43 and 48 of the VL as set forth in SEQ ID NO: 34. Alternatively or in addition, the transferrin receptor antibody of the present disclosure is a humanized antibody and contains the residues at positions 48, 67, 69, 71, and 73 of the VH as set forth in SEQ ID NO: 33.

The VH and VL amino acid sequences of an example humanized antibody that may be used in accordance with the present disclosure are provided:

Humanized VH
(SEQ ID NO: 35)
EVQLVQSGAEVKKPGASVKVSCKASGYTFTSYWMH
WVRQAPGQRLEWIGEINPTNGRTNYIEKFKSRATL
TVDKSASTAYMELSSLRSEDTAVYYCARGTRAYHY
WGQGTMVTVSS
Humanized VL
(SEQ ID NO: 36)
DIQMTQSPSSLSASVGDRVTITCRASDNLYSNLAWY
QQKPGKSPKLLVYDATNLADGVPSRFSGSGSGTDY
TLTISSLQPEDFATYYCQHFWGTPLTFGQGTKVEI
K

In some embodiments, the transferrin receptor antibody of the present disclosure comprises a VH comprising the amino acid sequence of SEQ ID NO: 35. Alternatively or in addition, the transferrin receptor antibody of the present disclosure comprises a VL comprising the amino acid sequence of SEQ ID NO: 36.

In some embodiments, the transferrin receptor antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 35. Alternatively or in addition, the transferrin receptor antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 36.

In some embodiments, the transferrin receptor antibody of the present disclosure comprises a VH comprising an amino acid sequence that is at least 80% (e.g., 80%, 85%, 90%, 95%, or 98%) identical to the VH as set forth in SEQ ID NO: 35. Alternatively or in addition, the transferrin receptor antibody of the present disclosure comprises a VL comprising an amino acid sequence that is at least 80% (e.g., 80%, 85%, 90%, 95%, or 98%) identical to the VL as set forth in SEQ ID NO: 36.

In some embodiments, the transferrin receptor antibody of the present disclosure is a humanized variant comprising amino acid substitutions at one or more of positions 43 and 48 as compared with the VL as set forth in SEQ ID NO: 34, and/or amino acid substitutions at one or more of positions 48, 67, 69, 71, and 73 as compared with the VH as set forth in SEQ ID NO: 33. In some embodiments, the transferrin receptor antibody of the present disclosure is a humanized variant comprising a S43A and/or a V48L mutation as compared with the VL as set forth in SEQ ID NO: 34, and/or one or more of A67V, L69I, V71R, and K73T mutations as compared with the VH as set forth in SEQ ID NO: 33

In some embodiments, the transferrin receptor antibody of the present disclosure is a humanized variant comprising amino acid substitutions at one or more of positions 9, 13, 17, 18, 40, 43, 48, 45, and 70 as compared with the VL as set forth in SEQ ID NO: 34, and/or amino acid substitutions at one or more of positions 1, 5, 7, 11, 12, 20, 38, 40, 44, 48, 66, 67, 69, 71, 73, 75, 81, 83, 87, and 108 as compared with the VH as set forth in SEQ ID NO: 33.

In some embodiments, the transferrin receptor antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the transferrin receptor antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the heavy chain of any of the transferrin receptor antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An exemplary human IgG1 constant region is given below:

(SEQ ID NO: 37)
ASTKGPSVFPLAPSSKSTSGGTAALGCLVKDYFPE
PVTVSWNSGALTSGVHTFPAVLQSSGLYSLSSVVT
VPSSSLGTQTYICNVNHKPSNTKVDKKVEPKSCDK
THTCPPCPAPELLGGPSVFLFPPKPKDTLMISRTP
EVTCVVVDVSHEDPEVKFNWYVDGVEVHNAKTKPR
EEQYNSTYRVVSVLTVLHQDWLNGKEYKCKVSNKA
LPAPIEKTISKAKGQPREPQVYTLPPSRDELTKNQ
VSLTCLVKGFYPSDIAVEWESNGQPENNYKTTPPV
LDSDGSFFLYSKLTVDKSRWQQGNVFSCSVMHEAL
HNHYTQKSLSLSPGK

In some embodiments, the light chain of any of the transferrin receptor antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 38)
RTVAAPSVFIFPPSDEQLKSGTASVVCLLNNFYPR
EAKVQWKVDNALQSGNSQESVTEQDSKDSTYSLSS
TLTLSKADYEKHKVYACEVTHQGLSSPVTKSFNRG
EC

Other antibody heavy and light chain constant regions are well known in the art, e.g., those provided in the IMGT database (www.imgt.org) or at www.vbase2.org/vbstat.php., both of which are incorporated by reference herein.

Exemplary heavy chain and light chain amino acid sequences of the transferrin receptor antibodies described are provided below:

Heavy Chain (VH + human IgG1
constant region)
(SEQ ID NO: 39)
QVQLQQPGAELVKPGASVKLSCKASGYTFTSYWMH
WVKQRPGQGLEWIGEINPTNGRTNYIEKFKSKATL
TVDKSSSTAYMQLSSLTSEDSAVYYCARGTRAYHY
WGQGTSVTVSSASTKGPSVFPLAPSSKSTSGGTAA
LGCLVKDYFPEPVTVSWNSGALTSGVHTFPAVLQS
SGLYSLSSVVTVPSSSLGTQTYICNVNHKPSNTKV
DKKVEPKSCDKTHTCPPCPAPELLGGPSVFLFPPK
PKDTLMISRTPEVTCVVVDVSHEDPEVKFNWYVDG
VEVHNAKTKPREEQYNSTYRVVSVLTVLHQDWLNG
KEYKCKVSNKALPAPIEKTISKAKGQPREPQVYTL
PPSRDELTKNQVSLTCLVKGFYPSDIAVEWESNGQ
PENNYKTTPPVLDSDGSFFLYSKLTVDKSRWQQGN
VFSCSVMHEALHNHYTQKSLSLSPGK
Light Chain (VL + kappa light chain)
(SEQ ID NO: 40)
DIQMTQSPASLSVSVGETVTITCRASDNLYSNLAW
YQQKQGKSPQLLVYDATNLADGVPSRFSGSGSGTQ
YSLKINSLQSEDFGTYYCQHFWGTPLTFGAGTKLE
LKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
Heavy Chain (humanized VH + human
IgG1 constant region)
(SEQ ID NO: 41)
EVQLVQSGAEVKKPGASVKVSCKASGYTFTSYWMH
WVRQAPGQRLEWIGEINPTNGRTNYIEKFKSRATL
TVDKSASTAYMELSSLRSEDTAVYYCARGTRAYHY
WGQGTMVTVSSASTKGPSVFPLAPSSKSTSGGTAA
LGCLVKDYFPEPVTVSWNSGALTSGVHTFPAVLQS
SGLYSLSSVVTVPSSSLGTQTYICNVNHKPSNTKV
DKKVEPKSCDKTHTCPPCPAPELLGGPSVFLFPPK
PKDTLMISRTPEVTCVVVDVSHEDPEVKFNWYVDG
VEVHNAKTKPREEQYNSTYRVVSVLTVLHQDWLNG
KEYKCKVSNKALPAPIEKTISKAKGQPREPQVYTL
PPSRDELTKNQVSLTCLVKGFYPSDIAVEWESNGQ
PENNYKTTPPVLDSDGSFFLYSKLTVDKSRWQQGN
VFSCSVMHEALHNHYTQKSLSLSPGK
Light Chain (humanized VL + kappa
light chain)
(SEQ ID NO: 42)
DIQMTQSPSSLSASVGDRVTITCRASDNLYSNLAW
YQQKPGKSPKLLVYDATNLADGVPSRFSGSGSGTD
YTLTISSLQPEDFATYYCQHFWGTPLTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC

In some embodiments, the transferrin receptor antibody described herein comprises a heavy chain comprising an amino acid sequence that is at least 80% (e.g., 80%, 85%, 90%, 95%, or 98%) identical to SEQ ID NO: 39. Alternatively or in addition, the transferrin receptor antibody described herein comprises a light chain comprising an amino acid sequence that is at least 80% (e.g., 80%, 85%, 90%, 95%, or 98%) identical to SEQ ID NO: 40. In some embodiments, the transferrin receptor antibody described herein comprises a heavy chain comprising the amino acid sequence of SEQ ID NO: 39. Alternatively or in addition, the transferrin receptor antibody described herein comprises a light chain comprising the amino acid sequence of SEQ ID NO: 40.

In some embodiments, the transferrin receptor antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 39. Alternatively or in addition, the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 40.

In some embodiments, the transferrin receptor antibody described herein comprises a heavy chain comprising an amino acid sequence that is at least 80% (e.g., 80%, 85%, 90%, 95%, or 98%) identical to SEQ ID NO: 41. Alternatively or in addition, the transferrin receptor antibody described herein comprises a light chain comprising an amino acid sequence that is at least 80% (e.g., 80%, 85%, 90%, 95%, or 98%) identical to SEQ ID NO: 42. In some embodiments, the transferrin receptor antibody described herein comprises a heavy chain comprising the amino acid sequence of SEQ ID NO: 41. Alternatively or in addition, the transferrin receptor antibody described herein comprises a light chain comprising the amino acid sequence of SEQ ID NO: 42.

In some embodiments, the transferrin receptor antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain of humanized antibody as set forth in SEQ ID NO: 39. Alternatively or in addition, the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain of humanized antibody as set forth in SEQ ID NO: 40.

In some embodiments, the transferrin receptor antibody is an antigen binding fragment (FAB) of an intact antibody (full-length antibody). Antigen binding fragment of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Exemplary FABs amino acid sequences of the transferrin receptor antibodies described herein are provided below:

(SEQ ID NO: 43)
Heavy Chain FAB (VH + a portion
of human IgG1 constant region)
QVQLQQPGAELVKPGASVKLSCKASGYTFTSYWMH
WVKQRPGQGLEWIGEINPTNGRTNYIEKFKSKATL
TVDKSSSTAYMQLSSLTSEDSAVYYCARGTRAYHY
WGQGTSVTVSSASTKGPSVFPLAPSSKSTSGGTAA
LGCLVKDYFPEPVTVSWNSGALTSGVHTFPAVLQS
SGLYSLSSVVTVPSSSLGTQTYICNVNHKPSNTKV
DKKVEPKSCDKTHTCP
Heavy Chain FAB (humanized VH + a
portion of human IgG1 constant region)
(SEQ ID NO: 44)
EVQLVQSGAEVKKPGASVKVSCKASGYTFTSYWMH
WVRQAPGQRLEWIGEINPTNGRTNYIEKFKSRATL
TVDKSASTAYMELSSLRSEDTAVYYCARGTRAYHY
WGQGTMVTVSSASTKGPSVFPLAPSSKSTSGGTAA
LGCLVKDYFPEPVTVSWNSGALTSGVHTFPAVLQS
SGLYSLSSVVTVPSSSLGTQTYICNVNHKPSNTKV
DKKVEPKSCDKTHTCP

The transferrin receptor antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, the transferrin receptor antibody described herein is a scFv. In some embodiments, the transferrin receptor antibody described herein is a scFv-Fab (e.g., scFv fused to a portion of a constant region). In some embodiments, the transferrin receptor antibody described herein is a scFv fused to a constant region (e.g., human IgG1 constant region as set forth in SEQ ID NO: 39).

b. Other Muscle-Targeting Antibodies

In some embodiments, the muscle-targeting antibody is an antibody that specifically binds hemojuvelin, caveolin-3, Duchenne muscular dystrophy peptide, myosin Jib or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered Clq binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like Cx or Ck. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g. a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 August 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 6) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 6). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for DMD. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 7) was identified and this muscle-targeting peptide showed improved binding to C2C12 cells relative to the ASSLNIA (SEQ ID NO: 6) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 8) appeared most frequently. Accordingly, in some embodiments, the muscle-targeting agent comprises the amino acid sequence TARGEHKEEELI (SEQ ID NO: 8).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081; Samoylova, T. I. and Smith, B. F. “Elucidation of muscle-binding peptides by phage display screening.” Muscle Nerve, 1999, 22:4. 460-6). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7; Zhang, L. “Molecular profiling of heart endothelial cells.” Circulation, 2005, 112:11, 1601-11; McGuire, M. J. et al. “In vitro selection of a peptide with high selectivity for cardiomyocytes in vivo.” J Mol Biol. 2004, 342:1, 171-82). Exemplary muscle-targeting peptides comprise an amino acid sequence of the following group: CQAQGQLVC (SEQ ID NO: 9), CSERSMNFC (SEQ ID NO: 10), CPKTRRVPC (SEQ ID NO: 11), WLSEAGPVVTVRALRGTGSW (SEQ ID NO: 12), ASSLNIA (SEQ ID NO: 6), CMQHSMRVC (SEQ ID NO: 13), and DDTRHWG (SEQ ID NO: 14). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147). A muscle-targeting agent may be an aptamer, e.g. an peptide aptamer, which preferentially targets muscle cells relative to other cell types.

iii. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

B. Molecular Payloads

Some aspects of the disclosure provide molecular payloads, e.g., for modulating a biological outcome, e.g., the transcription of a DNA sequence, the expression of a protein, or the activity of a protein. In some embodiments, a molecular payload is linked to, or otherwise associated with a muscle-targeting agent. It should be appreciated that various types of muscle-targeting agents may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, such molecular payloads are capable of targeting to a muscle cell, e.g., via specifically binding to a nucleic acid or protein in the muscle cell following delivery to the muscle cell by an associated muscle-targeting agent. In some embodiments, the molecular payload is an oligonucleotide that comprises a strand having a region of complementarity to a gene provided in Table 1. Exemplary molecular payloads are described in further detail herein, however, it should be appreciated that the exemplary molecular payloads provided herein are not meant to be limiting.

In some embodiments at least one (e.g., at least 2, at least 3, at least 4, at least 5, at least 10) molecular payload (e.g., oligonucleotides) is linked to a muscle-targeting agent. In some embodiments, all molecular payloads attached to a muscle-targeting agent are the same, e.g. target the same gene. In some embodiments, all molecular payloads attached to a muscle-targeting agent are different, for example the molecular payloads may target different portions of the same target gene, or the molecular payloads may target at least two different target genes. In some embodiments, a muscle-targeting agent may be attached to some molecular payloads that are the same and some molecular payloads that are different.

The present disclosure also provides a composition comprising a plurality of complexes, for which at least 80% (e.g., at least 85%, at least 90%, at least 91%, at least 92%, at least 93%, at least 94%, at least 95%, at least 96%, at least 97%, at least 98%, or at least 99%) of the complexes comprise a muscle-targeting agent linked to the same number of molecular payloads (e.g., oligonucleotides).

i. Oligonucleotides

Any suitable oligonucleotide may be used as a molecular payload, as described herein. In some embodiments, the oligonucleotide may be designed to cause degradation of an mRNA (e.g., the oligonucleotide may be a gapmer, an siRNA, a ribozyme or an aptamer that causes degradation). In some embodiments, the oligonucleotide may be designed to block translation of an mRNA (e.g., the oligonucleotide may be a mixmer, an siRNA or an aptamer that blocks translation). In some embodiments, an oligonucleotide may be designed to caused degradation and block translation of an mRNA. In some embodiments, an oligonucleotide may be a guide nucleic acid (e.g., guide RNA) for directing activity of an enzyme (e.g., a gene editing enzyme). Other examples of oligonucleotides are provided herein. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

In some embodiments, an oligonucleotide may comprise a region of complementarity to a target gene provided in Table 1. Further non-limiting examples are provided below for selected genes of Table 1.

DMPK/DM1

In some embodiments, examples of oligonucleotides useful for targeting DMPK, e.g., for the treatment of DM1, are provided in US Patent Application Publication 20100016215A1, published on Jan. 1, 2010, entitled Compound And Method For Treating Myotonic Dystrophy; US Patent Application Publication 20130237585A1, published Jul. 19, 2010, Modulation Of Dystrophia Myotonica-Protein Kinase (DMPK) Expression; US Patent Application Publication 20150064181A1, published on Mar. 5, 2015, entitled “Antisense Conjugates For Decreasing Expression Of Dmpk”; US Patent Application Publication 20150238627A1, published on Aug. 27, 2015, entitled “Peptide-Linked Morpholino Antisense Oligonucleotides For Treatment Of Myotonic Dystrophy”; Pandey, S. K. et al. “Identification and Characterization of Modified Antisense Oligonucleotides Targeting DMPK in Mice and Nonhuman Primates for the Treatment of Myotonic Dystrophy Type 1” J. of Pharmacol Exp Ther, 2015, 355:329-340; Langlois, M. et al. “Cytoplasmic and Nuclear Retained DMPK mRNAs Are Targets for RNA Interference in Myotonic Dystrophy Cells” J. Biological Chemistry, 2005, 280:17, 16949-16954; Jauvin, D. et al. “Targeting DMPK with Antisense Oligonucleotide Improves Muscle Strength in Myotonic Dystrophy Type 1 Mice”, Mol. Ther: Nucleic Acids, 2017, 7:465-474; Mulders, S. A. et al. “Triplet-repeat oligonucleotide-mediated reversal of RNA toxicity in myotonic dystrophy” PNAS, 2009, 106:33, 13915-13920; Wheeler, T. M. et al., “Targeting nuclear RNA for in vivo correction of myotonic dystrophy” Nature, 2012, 488(7409):111-115; and US Patent Application Publication 20160304877A1, published on Oct. 20, 2016, entitled “Compounds And Methods For Modulation Of Dystrophia Myotonica-Protein Kinase (Dmpk) Expression,” the contents of each of which are incorporated herein by reference in their entireties.

Examples of oligonucleotides for promoting DMPK gene editing include US Patent Application Publication 20170088819A1, published on Mar. 3, 2017, entitled “Genetic Correction Of Myotonic Dystrophy Type 1”; and International Patent Application Publication WO18002812A1, published on Apr. 1, 2018, entitled “Materials And Methods For Treatment Of Myotonic Dystrophy Type 1 (DM1) And Other Related Disorders,” the contents of each of which are incorporated herein by reference in their entireties.

In some embodiments, the oligonucleotide may have region of complementarity to a mutant form of DMPK, for example, a mutant form as reported in Botta A. et al. “The CTG repeat expansion size correlates with the splicing defects observed in muscles from myotonic dystrophy type 1 patients.” J Med Genet. 2008 October; 45(10):639-46; and Machuca-Tzili L. et al. “Clinical and molecular aspects of the myotonic dystrophies: a review.” Muscle Nerve. 2005 July; 32(1):1-18; the contents of each of which are incorporated herein by reference in their entireties.

In some embodiments, an oligonucleotide provided herein is an antisense oligonucleotide targeting DMPK. In some embodiments, the oligonucleotide targeting is any one of the antisense oligonucleotides (e.g., a Gapmer) targeting DMPK as described in US Patent Application Publication US20160304877A1, published on Oct. 20, 2016, entitled “Compounds And Methods For Modulation Of Dystrophia Myotonica-Protein Kinase (DMPK) Expression,” incorporated herein by reference. In some embodiments, the DMPK targeting oligonucleotide targets a region of the DMPK gene sequence as set forth in Genbank accession No. NM_001081560.2 or as set forth in Genbank accession No. NG_009784.1.

In some embodiments, the DMPK targeting oligonucleotide comprises a nucleotide sequence comprising a region complementary to a target region that is at least 10 continuous nucleotides (e.g., at least 10, at least 12, at least 14, at least 16, or more continuous nucleotides) in Genbank accession No. NM_001081560.2.

In some embodiments, the DMPK targeting oligonucleotide comprise a gapmer motif. “Gapmer” means a chimeric antisense compound in which an internal region having a plurality of nucleotides that support RNase H cleavage is positioned between external regions having one or more nucleotides, wherein the nucleotides comprising the internal region are chemically distinct from the nucleotide or nucleotides comprising the external regions. The internal region can be referred to as a “gap segment” and the external regions can be referred to as “wing segments.” In some embodiments, the DMPK targeting oligonucleotide comprises one or more modified nucleotides, and/or one or more modified internucleotide linkages. In some embodiments, the internucleotide linkage is a phosphorothioate linkage. In some embodiments, the oligonucleotide comprises a full phosphorothioate backbone. In some embodiments, the oligonucleotide is a DNA gapmer with cET ends (e.g., 3-10-3; cET-DNA-cET). In some embodiments, the DMPK targeting oligonucleotide comprises one or more 6′-(S)—CH₃ biocyclic nucleotides, one or more β-D-2′-deoxyribonucleotides, and/or one or more 5-methylcytosine nucleotides.

DUX4/FSHD

In some embodiments, examples of oligonucleotides useful for targeting DUX4, e.g., for the treatment of FSHD, are provided in U.S. Pat. No. 9,988,628, published on Feb. 2, 2017, entitled “AGENTS USEFUL IN TREATING FACIOSCAPULOHUMERAL MUSCULAR DYSTROPHY”; U.S. Pat. No. 9,469,851, published Oct. 30, 2014, entitled “RECOMBINANT VIRUS PRODUCTS AND METHODS FOR INHIBITING EXPRESSION OF DUX4”; US Patent Application Publication 20120225034, published on Sep. 6, 2012, entitled “AGENTS USEFUL IN TREATING FACIOSCAPULOHUMERAL MUSCULAR DYSTROPHY”; PCT Patent Application Publication Number WO 2013/120038, published on Aug. 15, 2013, entitled “MORPHOLINO TARGETING DUX4 FOR TREATING FSHD”; Chen et al., “Morpholino-mediated Knockdown of DUX4 Toward Facioscapulohumeral Muscular Dystrophy Therapeutics,” Molecular Therapy, 2016, 24:8, 1405-1411; and Ansseau et al., “Antisense Oligonucleotides Used to Target the DUX4 mRNA as Therapeutic Approaches in Facioscapulohumeral Muscular Dystrophy (FSHD),” Genes, 2017, 8, 93; the contents of each of which are incorporated herein in their entireties. In some embodiments, the oligonucleotide is an antisense oligonucleotide, a morpholino, a siRNA, a shRNA, or another nucleotide which hybridizes with the target DUX4 gene or mRNA.

In some embodiments, e.g., for the treatment of FSHD, oligonucleotides may have a region of complementarity to a hypomethylated, contracted D4Z4 repeat, as in Daxinger, et al., “Genetic and Epigenetic Contributors to FSHD,” published in Curr Opin Genet Dev in 2015, Lim J-W, et al., DICER/AGO-dependent epigenetic silencing of D4Z4 repeats enhanced by exogenous siRNA suggests mechanisms and therapies for FSHD Hum Mol Genet. 2015 Sep. 1; 24(17): 4817-4828, the contents of each of which are incorporated in their entireties.

DNM2/CNM

In some embodiments, examples of oligonucleotides useful for targeting DNM2, e.g., for the treatment of CNM, are provided in US Patent Application Publication Number 20180142008, published on May 24, 2018, entitled “DYNAMIN 2 INHIBITOR FOR THE TREATMENT OF DUCHENNE'S MUSCULAR DYSTROPHY”, and in PCT Application Publication Number WO 2018/100010A1, published on Jun. 7, 2018, entitled “ALLELE-SPECIFIC SILENCING THERAPY FOR DYNAMIN 2-RELATED DISEASES”. For example, in some embodiments, the oligonucleotide is a RNAi, an antisense nucleic acid, a siRNA, or a ribozyme that interferes specifically with DNM2 expression. Other examples of oligonucleotides useful for targeting DNM2 are provided in Tasfaout, et al., “Single Intramuscular Injection of AAV-shRNA Reduces DNM2 and Prevents Myotubular Myopathy in Mice,” published in Mol. Ther. on Apr. 4, 2018, and in Tasfaout, et al., “Antisense oligonucleotide-mediated Dnm2 knockdown prevents and reverts myotubular myopathy in mice,” Nature Communications volume 8, Article number: 15661 (2017). In some embodiments, the oligonucleotide is a shRNA or a morpholino that efficiently targets DNM2 mRNA. In some embodiments, the oligonucleotide encodes wild-type DNM2 which is resistant to miR-133 activity, as in Todaka, et al. “Overexpression of NF90-NF45 Represses Myogenic MicroRNA Biogenesis, Resulting in Development of Skeletal Muscle Atrophy and Centronuclear Muscle Fibers,” published in Mol. Cell Biol. in July 2015 Further examples of oligonucleotides useful for targeting DNM2 are provided in Gibbs, et al., “Two Dynamin-2 Genes are Required for Normal Zebrafish Development” published in PLoS One in 2013, the contents of each of which are incorporated herein in their entirety.

In some embodiments, e.g., for the treatment of CNM, the oligonucleotide may have a region of complementarity to a mutant in DNM2 associated with CNM, as in Böhm et al, “Mutation Spectrum in the Large GTPase Dynamin 2, and Genotype-Phenotype Correlation in Autosomal Dominant Centronuclear Myopathy,” as published in Hum. Mutat. in 2012, the contents of which are incorporated herein in its entirety.

Pompe Disease

In some embodiments, e.g., for the treatment of Pompe disease, an oligonucleotide mediates exon 2 inclusion in a GAA disease allele as in van der Wal, et al., “GAA Deficiency in Pompe Disease is Alleviated by Exon Inclusion in iPSC-Derived Skeletal Muscle Cells,” Mol Ther Nucleic Acids. 2017 Jun. 16; 7: 101-115, the contents of which are incorporated herein by reference. Accordingly, in some embodiments, the oligonucleotide may have a region of complementarity to a GAA disease allele.

In some embodiments, e.g., for the treatment of Pompe disease, an oligonucleotide, such as an RNAi or antisense oligonucleotide, is utilized to suppress expression of wild-type GYS1 in muscle cells, as reported, for example, in Clayton, et al., “Antisense Oligonucleotide-mediated Suppression of Muscle Glycogen Synthase 1 Synthesis as an Approach for Substrate Reduction Therapy of Pompe Disease,” published in Mol Ther Nucleic Acids in 2017, or US Patent Application Publication Number 2017182189, published on Jun. 29, 2017, entitled “INHIBITING OR DOWNREGULATING GLYCOGEN SYNTHASE BY CREATING PREMATURE STOP CODONS USING ANTISENSE OLIGONUCLEOTIDES”, the contents of which are incorporated herein by reference. Accordingly, in some embodiments, oligonucleotides may have an antisense strand having a region of complementarity to a sequence a human GYS1 sequence, corresponding to RefSeq number NM_002103.4 and/or a mouse GYS1 sequence, corresponding to RefSeq number NM_030678.3.

ACVR1/FOP

In some embodiments, examples of oligonucleotides useful for targeting ACVR1, e.g., for the treatment of FOP, are provided in US Patent Application 2009/0253132, published Oct. 8, 2009, “Mutated ACVR1 for diagnosis and treatment of fibrodyplasia ossificans progressiva (FOP)”; WO 2015/152183, published Oct. 8, 2015, “Prophylactic agent and therapeutic agent for fibrodysplasia ossificans progressive”; Lowery, J. W. et al, “Allele-specific RNA Interference in FOP—Silencing the FOP gene”, GENE THERAPY, vol. 19, 2012, pages 701-702; Takahashi, M. et al. “Disease-causing allele-specific silencing against the ALK2 mutants, R206H and G356D, in fibrodysplasia ossificans progressiva” Gene Therapy (2012) 19, 781-785; Shi, S. et al. “Antisense-Oligonucleotide Mediated Exon Skipping in Activin-Receptor-Like Kinase 2: Inhibiting the Receptor That Is Overactive in Fibrodysplasia Ossificans Progressiva” Plos One, July 2013, Vol 8:7, e69096; US Patent Application 2017/0159056, published Jun. 8, 2017, “Antisense oligonucleotides and methods of use thereof”; U.S. Pat. No. 8,859,752, issued Oct. 4, 2014, “SIRNA-based therapy of Fibrodyplasia Ossificans Progressiva (FOP)”; WO 2004/094636, published Nov. 4, 2004, “Effective sirna knock-down constructs”, the contents of each of which are incorporated herein in their entireties.

FXN/Friedreich's Ataxia

In some embodiments, examples of oligonucleotides useful for targeting FXN and/or otherwise compensating for frataxin deficiency, e.g., for the treatment of Freidrich Ataxia, are provided in Li, L. et al “Activating frataxin expression by repeat-targeted nucleic acids” Nat. Comm. 2016, 7:10606; WO 2016/094374, published Jun. 16, 2016, “Compositions and methods for treatment of friedreich's ataxia.”; WO 2015/020993, published Feb. 12, 2015, “RNAi COMPOSITIONS AND METHODS FOR TREATMENT OF FRIEDREICH'S ATAXIA”; WO 2017/186815, published Nov. 2, 2017, “Antisense oligonucleotides for enhanced expression of frataxin”; WO 2008/018795, published Feb. 14, 2008, “Methods and means for treating dna repeat instability associated genetic disorders”; US Patent Application 2018/0028557, published Feb. 1, 2018, “Hybrid oligonucleotides and uses thereof”; WO 2015/023975, published Feb. 19, 2015, “Compositions and methods for modulating RNA”; WO 2015/023939, published Feb. 19, 2015, “Compositions and methods for modulating expression of frataxin”; US Patent Application 2017/0281643, published Oct. 5, 2017, “Compounds and methods for modulating frataxin expression”; Li L. et al., “Activating frataxin expression by repeat-targeted nucleic acids” Nature Communications, Published 4 Feb. 2016; and Li L. et al. “Activation of Frataxin Protein Expression by Antisense Oligonucleotides Targeting the Mutant Expanded Repeat” Nucleic Acid Ther. 2018 February; 28(1):23-33, the contents of each of which are incorporated herein in their entireties.

In some embodiments, an oligonucleotide payload is configured (e.g., as a gapmer or RNAi oligonucleotide) for inhibiting expression of a natural antisense transcript that inhibits FXN expression, e.g., as disclosed in U.S. Pat. No. 9,593,330, filed Jun. 9, 2011, “Treatment of frataxin (FXN) related diseases by inhibition of natural antisense transcript to FXN”, the contents of which are incorporated herein by reference in its entirety.

Examples of oligonucleotides for promoting FXN gene editing include WO 2016/094845, published Jun. 16, 2016, “Compositions and methods for editing nucleic acids in cells utilizing oligonucleotides”; WO 2015/089354, published Jun. 18, 2015, “Compositions and methods of use of CRISPR-Cas systems in nucleotide repeat disorders”; WO 2015/139139, published Sep. 24, 2015, “CRISPR-based methods and products for increasing frataxin levels and uses thereof”; and WO 2018/002783, published Jan. 4, 2018, “Materials and methods for treatment of Friedreich ataxia and other related disorders”, the contents of each of which are incorporated herein in their entireties.

Examples of oligonucleotides for promoting FXN gene expression through targeting of non-FXN genes, e.g. epigenetic regulators of FXN, include WO 2015/023938, published Feb. 19, 2015, “Epigenetic regulators of frataxin”, the contents of which are incorporated herein in its entirety.

In some embodiments, oligonucleotides may have a region of complementarity to a sequence set forth as: a FXN gene from humans (Gene ID 2395; NC_000009.12) and/or a FXN gene from mice (Gene ID 14297; NC_000085.6). In some embodiments, the oligonucleotide may have region of complementarity to a mutant form of FXN, for example as reported in e.g., Montermini, L. et al. “The Friedreich ataxia GAA triplet repeat: premutation and normal alleles.” Hum. Molec. Genet., 1997, 6: 1261-1266; Filla, A. et al. “The relationship between trinucleotide (GAA) repeat length and clinical features in Friedreich ataxia.” Am. J. Hum. Genet. 1996, 59: 554-560; Pandolfo, M. Friedreich ataxia: the clinical picture. J. Neurol. 2009, 256, 3-8; the contents of each of which are incorporated herein by reference in their entireties.

DMD/Dystrophinopathies

Examples of oligonucleotides useful for targeting DMD are provided in U.S. Patent Application Publication US20100130591A1, published on May 27, 2010, entitled “MULTIPLE EXON SKIPPING COMPOSITIONS FOR DMD”; U.S. Pat. No. 8,361,979, issued Jan. 29, 2013, entitled “MEANS AND METHOD FOR INDUCING EXON-SKIPPING”; U.S. Patent Application Publication 20120059042, published Mar. 8, 2012, entitled “METHOD FOR EFFICIENT EXON (44) SKIPPING IN DUCHENNE MUSCULAR DYSTROPHY AND ASSOCIATED MEANS; U.S. Patent Application Publication 20140329881, published Nov. 6, 2014, entitled “EXON SKIPPING COMPOSITIONS FOR TREATING MUSCULAR DYSTROPHY”; U.S. Pat. No. 8,232,384, issued Jul. 31, 2012, entitled “ANTISENSE OLIGONUCLEOTIDES FOR INDUCING EXON SKIPPING AND METHODS OF USE THEREOF”; U.S. Patent Application Publication 20120022134A1, published Jan. 26, 2012, entitled “METHODS AND MEANS FOR EFFICIENT SKIPPING OF EXON 45 IN DUCHENNE MUSCULAR DYSTROPHY PRE-MRNA; U.S. Patent Application Publication 20120077860, published Mar. 29, 2012, entitled “ADENO-ASSOCIATED VIRAL VECTOR FOR EXON SKIPPING IN A GENE ENCODING A DISPENSABLE DOMAN PROTEIN”; U.S. Pat. No. 8,324,371, issued Dec. 4, 2012, entitled “OLIGOMERS”; U.S. Pat. No. 9,078,911, issued Jul. 14, 2015, entitled “ANTISENSE OLIGONUCLEOTIDES”; U.S. Pat. No. 9,079,934, issued Jul. 14, 2015, entitled “ANTISENSE NUCLEIC ACIDS”; U.S. Pat. No. 9,034,838, issued May 19, 2015, entitled “MIR-31 IN DUCHENNE MUSCULAR DYSTROPHY THERAPY”; and International Patent Publication WO2017062862A3, published Apr. 13, 2017, entitled “OLIGONUCLEOTIDE COMPOSITIONS AND METHODS THEREOF”; the contents of each of which are incorporated herein in their entireties.

Examples of oligonucleotides for promoting DMD gene editing include International Patent Publication WO2018053632A1, published Mar. 29, 2018, entitled “METHODS OF MODIFYING THE DYSTROPHIN GENE AND RESTORING DYSTROPHIN EXPRESSION AND USES THEREOF”; International Patent Publication WO2017049407A1, published Mar. 30, 2017, entitled “MODIFICATION OF THE DYSTROPHIN GENE AND USES THEREOF”; International Patent Publication WO2016161380A1, published Oct. 6, 2016, entitled “CRISPR/CAS-RELATED METHODS AND COMPOSITIONS FOR TREATING DUCHENNE MUSCULAR DYSTROPHY AND BECKER MUSCULAR DYSTROPHY”; International Patent Publication WO2017095967, published Jun. 8, 2017, entitled “THERAPEUTIC TARGETS FOR THE CORRECTION OF THE HUMAN DYSTROPHIN GENE BY GENE EDITING AND METHODS OF USE”; International Patent Publication WO2017072590A1, published May 4, 2017, entitled “MATERIALS AND METHODS FOR TREATMENT OF DUCHENNE MUSCULAR DYSTROPHY”; International Patent Publication WO2018098480A1, published May 31, 2018, entitled “PREVENTION OF MUSCULAR DYSTROPHY BY CRISPR/CPF1-MEDIATED GENE EDITING”; US Patent Application Publication US20170266320A1, published Sep. 21, 2017, entitled “RNA-Guided Systems for In Vivo Gene Editing”; International Patent Publication WO2016025469A1, published Feb. 18, 2016, entitled “PREVENTION OF MUSCULAR DYSTROPHY BY CRISPR/CAS9-MEDIATED GENE EDITING”; U.S. Patent Application Publication 2016/0201089, published Jul. 14, 2016, entitled “RNA-GUIDED GENE EDITING AND GENE REGULATION”; and U.S. Patent Application Publication 2013/0145487, published Jun. 6, 2013, entitled “MEGANUCLEASE VARIANTS CLEAVING A DNA TARGET SEQUENCE FROM THE DYSTROPHN GENE AND USES THEREOF”, the contents of each of which are incorporated herein in their entireties. In some embodiments, an oligonucleotide may have a region of complementarity to DMD gene sequences of multiple species, e.g., selected from human, mouse and non-human species.

In some embodiments, the oligonucleotide may have region of complementarity to a mutant DMD allele, for example, a DMD allele with at least one mutation in any of exons 1-79 of DMD in humans that leads to a frameshift and improper RNA splicing/processing.

MYH7/Hypertrophic Cardiomyopathy

Examples of oligonucleotides useful as payloads, e.g., for targeting MYH7 are provided in US Patent Application Publication 20180094262, published on Apr. 5, 2018, entitled Inhibitors of MYH7B and Uses Thereof; US Patent Application Publication 20160348103, published on Dec. 1, 2016, entitled Oligonucleotides and Methods for Treatment of Cardiomyopathy Using RNA Interference; US Patent Application Publication 20160237430, published on Aug. 18, 2016, entitled “Allele-specific RNA Silencing for the Treatment of Hypertrophic Cardiomyopathy”; US Patent Application Publication 20160032286, published on Feb. 4, 2016, entitled “Inhibitors of MYH7B and Uses Thereof”; US Patent Application Publication 20140187603, published on Jul. 3, 2014, entitled “MicroRNA Inhibitors Comprising Locked Nucleotides”; US Patent Application Publication 20140179764, published on Jun. 26, 2014, entitled “Dual Targeting of miR-208 and miR-499 in the Treatment of Cardiac Disorders”; US Patent Application Publication 20120114744, published on May 10, 2012, entitled “Compositions and Methods to Treat Muscular and Cardiovascular Disorders”; the contents of each of which are incorporated herein in their entireties.

In some embodiments, the oligonucleotide may target lncRNA or mRNA, e.g., for degradation. In some embodiments, the oligonucleotide may target, e.g., for degradation, a nucleic acid encoding a protein involved in a mismatch repair pathway, e.g., MSH2, MutLalpha, MutSbeta, MutLalpha. Non-limiting examples of proteins involved in mismatch repair pathways, for which mRNAs encoding such proteins may be targeted by oligonucleotides described herein, are described in Iyer, R. R. et al., “DNA triplet repeat expansion and mismatch repair” Annu Rev Biochem. 2015; 84:199-226; and Schmidt M. H. and Pearson C. E., “Disease-associated repeat instability and mismatch repair” DNA Repair (Amst). 2016 February; 38:117-26.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In a some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, etc.

In some embodiments, a complementary nucleic acid sequence of an oligonucleotide for purposes of the present disclosure is specifically hybridizable or specific for the target nucleic acid when binding of the sequence to the target molecule (e.g., mRNA) interferes with the normal function of the target (e.g., mRNA) to cause a loss of activity (e.g., inhibiting translation) or expression (e.g., degrading a target mRNA) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of an target nucleic acid. In some embodiments a complementary nucleotide sequence need not be 100% complementary to that of its target to be specifically hybridizable or specific for a target nucleic acid.

In some embodiments, an oligonucleotide comprises region of complementarity to a target nucleic acid that is in the range of 8 to 15, 8 to 30, 8 to 40, or 10 to 50, or 5 to 50, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

b. Oligonucleotide Modifications:

The oligonucleotides described herein may be modified, e.g., comprise a modified sugar moiety, a modified internucleoside linkage, a modified nucleotide and/or combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are modified nucleotides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are modified nucleotides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are modified nucleotides. Optionally, the oligonucleotides may have every nucleotide except 1, 2, 3, 4, 5, 6, 7, 8, 9, or 10 nucleotides modified. Oligonucleotide modifications are described further herein.

c. Modified Nucleotides

In some embodiments, an oligonucleotide include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some embodiments, an oligonucleotide can include at least one 2′-O-methyl-modified nucleotide, and in some embodiments, all of the nucleotides include a 2′-O-methyl modification. In some embodiments, an oligonucleotide comprises modified nucleotides in which the ribose ring comprises a bridge moiety connecting two atoms in the ring, e.g., connecting the 2′-O atom to the 4′-C atom. In some embodiments, the oligonucleotides are “locked,” e.g., comprise modified nucleotides in which the ribose ring is “locked” by a methylene bridge connecting the 2′-O atom and the 4′-C atom. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety.

Other modifications that may be used in the oligonucleotides disclosed herein include ethylene-bridged nucleic acids (ENAs). ENAs include, but are not limited to, 2′-0,4′-C-ethylene-bridged nucleic acids. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties.

In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. In some embodiments, the oligonucleotide comprises a modified nucleotide disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA.

In some embodiments, the oligonucleotide may have at least one modified nucleotide that results in an increase in Tm of the oligonucleotide in a range of 1° C., 2° C., 3° C., 4° C., or 5° C. compared with an oligonucleotide that does not have the at least one modified nucleotide. The oligonucleotide may have a plurality of modified nucleotides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleotide.

The oligonucleotide may comprise alternating nucleotides of different kinds. For example, an oligonucleotide may comprise alternating deoxyribonucleotides or ribonucleotides and 2′-fluoro-deoxyribonucleotides. An oligonucleotide may comprise alternating deoxyribonucleotides or ribonucleotides and 2′-O-methyl nucleotides. An oligonucleotide may comprise alternating 2′-fluoro nucleotides and 2′-O-methyl nucleotides. An oligonucleotide may comprise alternating bridged nucleotides and 2′-fluoro or 2′-O-methyl nucleotides.

d. Internucleotide Linkages/Backbones

In some embodiments, oligonucleotide may contain a phosphorothioate or other modified internucleotide linkage. In some embodiments, the oligonucleotide comprises phosphorothioate internucleoside linkages. In some embodiments, the oligonucleotide comprises phosphorothioate internucleoside linkages between at least two nucleotides. In some embodiments, the oligonucleotide comprises phosphorothioate internucleoside linkages between all nucleotides. For example, in some embodiments, oligonucleotides comprise modified internucleotide linkages at the first, second, and/or third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides are adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides are provided that comprise nucleoside units that are joined together by either substantially all Sp or substantially all Rp phosphorothioate intersugar linkages. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Gapmers

In some embodiments, the oligonucleotide is a gapmer. A gapmer oligonucleotide generally has the formula 5′-X-Y-Z-3′, with X and Z as flanking regions around a gap region Y. In some embodiments, the Y region is a contiguous stretch of nucleotides, e.g., a region of at least 6 DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleotides, e.g., one to six modified nucleotides. Examples of modified nucleotides include, but are not limited to, 2′ MOE or 2′OMe or Locked Nucleic Acid bases (LNA). The flanking sequences X and Z may be of one to twenty nucleotides, one to eight nucleotides or one to five nucleotides in length, in some embodiments. The flanking sequences X and Z may be of similar length or of dissimilar lengths. The gap-segment Y may be a nucleotide sequence of five to twenty nucleotides, size to twelve nucleotides or six to ten nucleotides in length, in some embodiments.

In some embodiments, the gap region of the gapmer oligonucleotides may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,432,250; and 7,683,036; U.S. patent publication Nos. US20090286969, US20100197762, and US20110112170; and PCT publication Nos. WO2008049085 and WO2009090182, each of which is herein incorporated by reference in its entirety.

i. Mixmers

In some embodiments, an oligonucleotide described herein may be a mixmer or comprise a mixmer sequence pattern. In general, mixmers are oligonucleotides that comprise both naturally and non-naturally occurring nucleotides or comprise two different types of non-naturally occurring nucleotides typically in an alternating pattern. Mixmers generally have higher binding affinity than unmodified oligonucleotides and may be used to specifically bind a target molecule, e.g., to block a binding site on the target molecule. Generally, mixmers do not recruit an RNAse to the target molecule and thus do not promote cleavage of the target molecule. Such oligonucleotides that are incapable of recruiting RNAse H have been described, for example, see WO2007/112754 or WO2007/112753.

In some embodiments, the mixmer comprises or consists of a repeating pattern of nucleotide analogues and naturally occurring nucleotides, or one type of nucleotide analogue and a second type of nucleotide analogue. However, a mixmer need not comprise a repeating pattern and may instead comprise any arrangement of modified nucleotides and naturally occurring nucleotides or any arrangement of one type of modified nucleotide and a second type of modified nucleotide. The repeating pattern, may, for instance be every second or every third nucleotide is a modified nucleotide, such as LNA, and the remaining nucleotides are naturally occurring nucleotides, such as DNA, or are a 2′ substituted nucleotide analogue such as 2′MOE or 2′ fluoro analogues, or any other modified nucleotide described herein. It is recognized that the repeating pattern of modified nucleotide, such as LNA units, may be combined with modified nucleotide at fixed positions—e.g. at the 5′ or 3′ termini.

In some embodiments, a mixmer does not comprise a region of more than 5, more than 4, more than 3, or more than 2 consecutive naturally occurring nucleotides, such as DNA nucleotides. In some embodiments, the mixmer comprises at least a region consisting of at least two consecutive modified nucleotide, such as at least two consecutive LNAs. In some embodiments, the mixmer comprises at least a region consisting of at least three consecutive modified nucleotide units, such as at least three consecutive LNAs.

In some embodiments, the mixmer does not comprise a region of more than 7, more than 6, more than 5, more than 4, more than 3, or more than 2 consecutive nucleotide analogues, such as LNAs. In some embodiments, LNA units may be replaced with other nucleotide analogues, such as those referred to herein.

Mixmers may be designed to comprise a mixture of affinity enhancing modified nucleotides, such as in non-limiting example LNA nucleotides and 2′-O-methyl nucleotides. In some embodiments, a mixmer comprises modified internucleoside linkages (e.g., phosphorothioate internucleoside linkages or other linkages) between at least two, at least three, at least four, at least five or more nucleotides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleotides. For example, in some embodiments, a mixmer may comprise morpholino nucleotides mixed (e.g., in an alternating manner) with one or more other nucleotides (e.g., DNA, RNA nucleotides) or modified nucleotides (e.g., LNA, 2′-O-Methyl nucleotides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′-O-Methyl Mixmer Antisense Oligonucleotide, Molecules 2016, 21, 1582, the contents of each which are incorporated herein by reference.

j. RNA Interference (RNAi)

In some embodiments, oligonucleotides provided herein may be in the form of small interfering RNAs (siRNA), also known as short interfering RNA or silencing RNA. SiRNA, is a class of double-stranded RNA molecules, typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective.

Following selection of an appropriate target RNA sequence, siRNA molecules that comprise a nucleotide sequence complementary to all or a portion of the target sequence, i.e. an antisense sequence, can be designed and prepared using appropriate methods (see, e.g., PCT Publication Number WO 2004/016735; and U.S. Patent Publication Nos. 2004/0077574 and 2008/0081791).

The siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

Double-stranded siRNA may comprise RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). A spacer sequence is may be an unrelated nucleotide sequence that is situated between two complementary nucleotide sequence regions which, when annealed into a double-stranded nucleic acid, comprise a shRNA.

The overall length of the siRNA molecules can vary from about 14 to about 100 nucleotides depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 100 nucleotides.

An siRNA molecule may comprise a 3′ overhang at one end of the molecule, The other end may be blunt-ended or have also an overhang (5′ or 3′). When the siRNA molecule comprises an overhang at both ends of the molecule, the length of the overhangs may be the same or different. In one embodiment, the siRNA molecule of the present disclosure comprises 3′ overhangs of about 1 to about 3 nucleotides on both ends of the molecule.

k. microRNA (miRNAs)

In some embodiments, an oligonucleotide may be a microRNA (miRNA). MicroRNAs (referred to as “miRNAs”) are small non-coding RNAs, belonging to a class of regulatory molecules that control gene expression by binding to complementary sites on a target RNA transcript. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer.

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides. In one embodiment the size range of the miRNA is from 70 to 170 nucleotides in length. In another embodiment, mature miRNAs of from 21 to 25 nucleotides in length can be used.

l. Aptamers

In some embodiments, oligonucleotides provided herein may be in the form of aptamers. Generally, in the context of molecular payloads, aptamer is any nucleic acid that binds specifically to a target, such as a small molecule, protein, nucleic acid in a cell. In some embodiments, the aptamer is a DNA aptamer or an RNA aptamer. In some embodiments, a nucleic acid aptamer is a single-stranded DNA or RNA (ssDNA or ssRNA). It is to be understood that a single-stranded nucleic acid aptamer may form helices and/or loop structures. The nucleic acid that forms the nucleic acid aptamer may comprise naturally occurring nucleotides, modified nucleotides, naturally occurring nucleotides with hydrocarbon linkers (e.g., an alkylene) or a polyether linker (e.g., a PEG linker) inserted between one or more nucleotides, modified nucleotides with hydrocarbon or PEG linkers inserted between one or more nucleotides, or a combination of thereof. Exemplary publications and patents describing aptamers and method of producing aptamers include, e.g., Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823; 6,569,630; 8,318,438 and PCT application WO 99/31275, each incorporated herein by reference.

m. Ribozymes

In some embodiments, oligonucleotides provided herein may be in the form of a ribozyme. A ribozyme (ribonucleic acid enzyme) is a molecule, typically an RNA molecule, that is capable of performing specific biochemical reactions, similar to the action of protein enzymes. Ribozymes are molecules with catalytic activities including the ability to cleave at specific phosphodiester linkages in RNA molecules to which they have hybridized, such as mRNAs, RNA-containing substrates, lncRNAs, and ribozymes, themselves.

Ribozymes may assume one of several physical structures, one of which is called a “hammerhead.” A hammerhead ribozyme is composed of a catalytic core containing nine conserved bases, a double-stranded stem and loop structure (stem-loop II), and two regions complementary to the target RNA flanking regions the catalytic core. The flanking regions enable the ribozyme to bind to the target RNA specifically by forming double-stranded stems I and III. Cleavage occurs in cis (i.e., cleavage of the same RNA molecule that contains the hammerhead motif) or in trans (cleavage of an RNA substrate other than that containing the ribozyme) next to a specific ribonucleotide triplet by a transesterification reaction from a 3′, 5′-phosphate diester to a 2′, 3′-cyclic phosphate diester. Without wishing to be bound by theory, it is believed that this catalytic activity requires the presence of specific, highly conserved sequences in the catalytic region of the ribozyme.

Modifications in ribozyme structure have also included the substitution or replacement of various non-core portions of the molecule with non-nucleotidic molecules. For example, Benseler et al. (J. Am. Chem. Soc. (1993) 115:8483-8484) disclosed hammerhead-like molecules in which two of the base pairs of stem II, and all four of the nucleotides of loop II were replaced with non-nucleoside linkers based on hexaethylene glycol, propanediol, bis(triethylene glycol) phosphate, tris(propanediol)bisphosphate, or bis(propanediol) phosphate. Ma et al. (Biochem. (1993) 32:1751-1758; Nucleic Acids Res. (1993) 21:2585-2589) replaced the six nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length.

Ribozyme oligonucleotides can be prepared using well known methods (see, e.g., PCT Publications WO9118624; WO9413688; WO9201806; and WO 92/07065; and U.S. Pat. Nos. 5,436,143 and 5,650,502) or can be purchased from commercial sources (e.g., US Biochemicals) and, if desired, can incorporate nucleotide analogs to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme may also be produced in recombinant vectors by conventional means. See, Molecular Cloning: A Laboratory Manual, Cold Spring Harbor Laboratory (Current edition). The ribozyme RNA sequences maybe synthesized conventionally, for example, by using RNA polymerases such as T7 or SP6.

n. Guide Nucleic Acids

In some embodiments, oligonucleotides are guide nucleic acid, e.g., guide RNA (gRNA) molecules. Generally, a guide RNA is a short synthetic RNA composed of (1) a scaffold sequence that binds to a nucleic acid programmable DNA binding protein (napDNAbp), such as Cas9, and (2) a nucleotide spacer portion that defines the DNA target sequence (e.g., genomic DNA target) to which the gRNA binds in order to bring the nucleic acid programmable DNA binding protein in proximity to the DNA target sequence. In some embodiments, the napDNAbp is a nucleic acid-programmable protein that forms a complex with (e.g., binds or associates with) one or more RNA(s) that targets the nucleic acid-programmable protein to a target DNA sequence (e.g., a target genomic DNA sequence). In some embodiments, a nucleic acid-programmable nuclease, when in a complex with an RNA, may be referred to as a nuclease:RNA complex. Guide RNAs can exist as a complex of two or more RNAs, or as a single RNA molecule.

Guide RNAs (gRNAs) that exist as a single RNA molecule may be referred to as single-guide RNAs (sgRNAs), though gRNA is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 protein. In some embodiments, domain (2) corresponds to a sequence known as a tracrRNA and comprises a stem-loop structure. In some embodiments, domain (2) is identical or homologous to a tracrRNA as provided in Jinek et al., Science 337:816-821 (2012), the entire contents of which is incorporated herein by reference.

In some embodiments, a gRNA comprises two or more of domains (1) and (2), and may be referred to as an extended gRNA. For example, an extended gRNA will bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csnl) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

o. Splice Altering Oligonucleotides

In some embodiments, a oligonucleotide (e.g., an antisense oligonucleotide including a morpholino) of the present disclosure target splicing. In some embodiments, the oligonucleotide targets splicing by inducing exon skipping and restoring the reading frame within a gene. As a non-limiting example, the oligonucleotide may induce skipping of an exon encoding a frameshift mutation and/or an exon that encodes a premature stop codon. In some embodiments, an oligonucleotide may induce exon skipping by blocking spliceosome recognition of a splice site. In some embodiments, exon skipping results in a truncated but functional protein compared to the reference protein (e.g., truncated but functional DMD protein as described below). In some embodiments, the oligonucleotide promotes inclusion of a particular exon (e.g., exon 7 of the SMN2 gene described below). In some embodiments, an oligonucleotide may induce inclusion of an exon by targeting a splice site inhibitory sequence. RNA splicing has been implicated in muscle diseases, including Duchenne muscular dystrophy (DMD) and spinal muscular atrophy (SMA).

Alterations (e.g., deletions, point mutations, and duplications) in the gene encoding dystrophin (DMD) cause DMD. These alterations can lead to frameshift mutations and/or nonsense mutations. In some embodiments, an oligonucleotide of the present disclosure promotes skipping of one or more DMD exons (e.g., exon 8, exon 43, exon 44, exon 45, exon 50, exon 51, exon 52, exon 53, and/or exon 55) and results in a functional truncated protein. See, e.g., U.S. Pat. No. 8,486,907 published on Jul. 16, 2013 and U.S. 20140275212 published on Sep. 18, 2014.

In SMA, there is loss of functional SMN1. Although the SMN2 gene is a paralog to SMN1, alternative splicing of the SMN2 gene predominantly leads to skipping of exon 7 and subsequent production of a truncated SMN protein that cannot compensate for SMN1 loss. In some embodiments, an oligonucleotide of the present disclosure promotes inclusion of SMN2 exon 7. In some embodiments, an oligonucleotide is an antisense oligonucleotide that targets SMN2 splice site inhibitory sequences (see, e.g., U.S. Pat. No. 7,838,657, which was published on Nov. 23, 2010).

p. Multimers

In some embodiments, molecular payloads may comprise multimers (e.g., concatemers) of 2 or more oligonucleotides connected by a linker. In this way, in some embodiments, the oligonucleotide loading of a complex/conjugate can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via a oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

C. Linkers

Complexes described herein generally comprise a linker that connects a muscle-targeting agent (e.g., muscle-targeting protein) to a molecular payload (e.g., oligonucleotide). A linker comprises at least one covalent bond. In some embodiments, a linker may be a single bond, e.g., a disulfide bond or disulfide bridge, that connects a muscle-targeting agent to a molecular payload. However, in some embodiments, a linker may connect a muscle-targeting agent to a molecular payload through multiple covalent bonds. In some embodiments, a linker may be a cleavable linker. However, in some embodiments, a linker may be a non-cleavable linker. A linker is generally stable in vitro and in vivo, and may be stable in certain cellular environments. Additionally, generally a linker does not negatively impact the functional properties of either the muscle-targeting agent or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493; Jain, N. et al. “Current ADC Linker Chemistry” Pharm Res. 2015, 32:11, 3526-3540; McCombs, J. R. and Owen, S. C. “Antibody Drug Conjugates: Design and Selection of Linker, Payload and Conjugation Chemistry” AAPS J. 2015, 17:2, 339-351).

A precursor to a linker typically will contain two different reactive species that allow for attachment to both the muscle-targeting agent and a molecular payload. In some embodiments, the two different reactive species may be a nucleophile and/or an electrophile. In some embodiments, a linker is connected to a muscle-targeting agent via conjugation to a lysine residue or a cysteine residue of the muscle-targeting agent. In some embodiments, a linker is connected to a cysteine residue of a muscle-targeting agent via a maleimide-containing linker, wherein optionally the maleimide-containing linker comprises a maleimidocaproyl or maleimidomethyl cyclohexane-1-carboxylate group. In some embodiments, a linker is connected to a cysteine residue of a muscle-targeting agent or thiol functionalized molecular payload via a 3-arylpropionitrile functional group. In some embodiments, a linker is connected to a muscle-targeting agent and/or a molecular payload via an amide bond, a hydrazide, a triazole, a thioether or a disulfide bond.

i. Cleavable Linkers

A cleavable linker may be a protease-sensitive linker, a pH-sensitive linker, or a glutathione-sensitive linker. These linkers are generally cleavable only intracellularly and are preferably stable in extracellular environments, e.g. extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include j-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline dipeptide sequence. In some embodiments, a protease-sensitive linker can be cleaved by a lysosomal protease, e.g. cathepsin B, and/or an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by an disulfide exchange reaction with a glutathione species inside a cell. In some embodiments, the disulfide moiety further comprises at least one amino acid, e.g. a cysteine residue. In some embodiments, a cleavable linker is a valine-citrulline linker.

In some embodiments, the linker is a Val-cit linker (e.g., as described in U.S. Pat. No. 6,214,345, incorporated herein by reference). In some embodiments, before conjugation, the val-cit linker has a structure of:

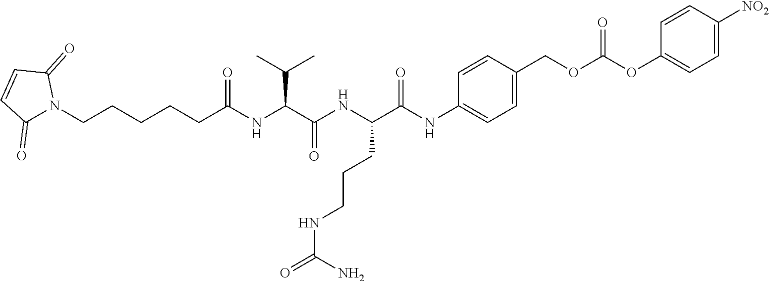 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, the val-cit linker has a structure of:

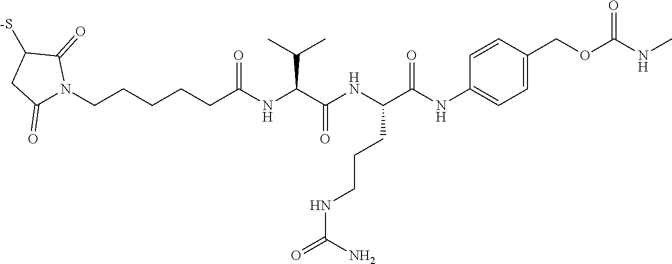

In some embodiments, before conjugation, the val-cit linker has a structure of:

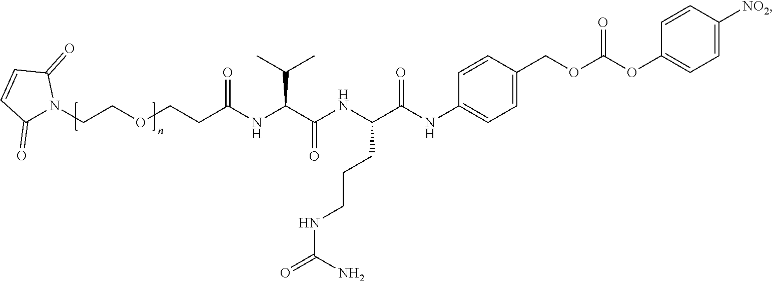

wherein n is any number from 0-10.

In some embodiments, before conjugation, the val-cit linker has a structure of:

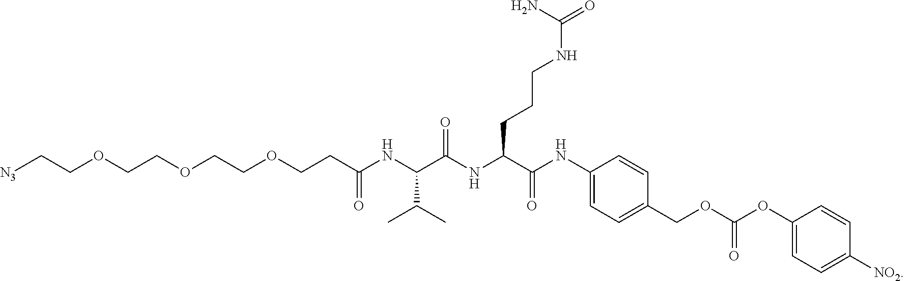 [SMILES: CC(C)[C@H](NC(=O)CCOCCOCCOCCN=[N+]=[N-])C(=O)N[C@@H](CCCNC(N)=O)C(=O)Nc1ccc(COC(=O)Oc2ccc([N+](=O)[O-])cc2)cc1]

In some embodiments, before conjugation, the val-cit linker has a structure of:

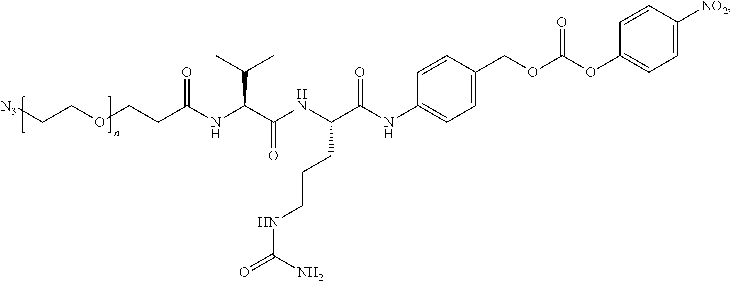

wherein n is any number from 0-10.

In some embodiments, after conjugation to an oligonucleotide, the val-cit linker has a structure of:

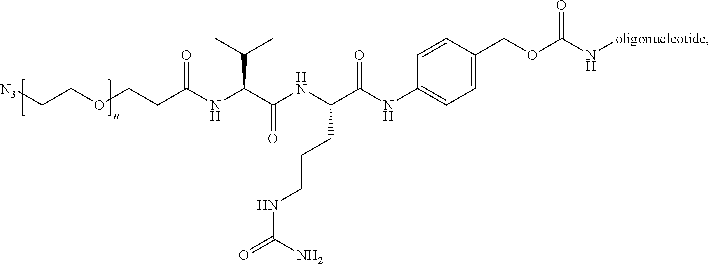

wherein n is any number from 0-10.

In some embodiments, after conjugation to an oligonucleotide, the val-cit linker has a structure of:

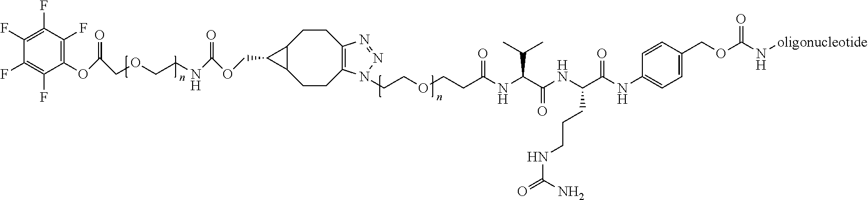

wherein n is any number from 0-10.

ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence (SEQ ID NO: 15), a thioether, a biotin, a biphenyl, repeating units of polyethylene glycol or equivalent compounds, acid esters, acid amides, sulfamides, and/or an alkoxy-amine linker. In some embodiments, sortase-mediated ligation will be utilized to covalently link a muscle-targeting agent comprising a LPXT sequence to a molecular payload comprising a (G)ₙ sequence (see, e.g. Proft T. Sortase-mediated protein ligation: an emerging biotechnology tool for protein modification and immobilization. Biotechnol Lett. 2010, 32(1):1-10). In some embodiments, a linker comprises a LPXTG sequence (SEQ ID NO: 16), where X is any amino acid.

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S; an imino, an optionally substituted nitrogen species, an optionally substituted oxygen species O, an optionally substituted sulfur species, or a poly(alkylene oxide), e.g. polyethylene oxide or polypropylene oxide.

iii. Linker Conjugation

In some embodiments, a linker is connected to a muscle-targeting agent and/or molecular payload via a phosphate, thioether, ether, carbon-carbon, or amide bond. In some embodiments, a linker is connected to an oligonucleotide through a phosphate or phosphorothioate group, e.g. a terminal phosphate of an oligonucleotide backbone. In some embodiments, a linker is connected to an muscle-targeting agent, e.g. an antibody, through a lysine or cysteine residue present on the muscle-targeting agent

In some embodiments, a linker is connected to a muscle-targeting agent and/or molecular payload by a cycloaddition reaction between an azide and an alkyne to form a triazole, wherein the azide and the alkyne may be located on the muscle-targeting agent, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctane is as described in International Patent Application Publication WO2011136645, published on Nov. 3, 2011, entitled, “Fused Cyclooctyne Compounds And Their Use In Metal-free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide and the alkyne may be located on the muscle-targeting agent, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker further comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is connected to a muscle-targeting agent and/or molecular payload by the Diels-Alder reaction between a dienophile and a diene/hetero-diene, wherein the dienophile and the diene/hetero-diene may be located on the muscle-targeting agent, molecular payload, or the linker. In some embodiments a linker is connected to a muscle-targeting agent and/or molecular payload by other pericyclic reactions, e.g. ene reaction. In some embodiments, a linker is connected to a muscle-targeting agent and/or molecular payload by an amide, thioamide, or sulfonamide bond reaction. In some embodiments, a linker is connected to a muscle-targeting agent and/or molecular payload by a condensation reaction to form an oxime, hydrazone, or semicarbazide group existing between the linker and the muscle-targeting agent and/or molecular payload.

In some embodiments, a linker is connected to a muscle-targeting agent and/or molecular payload by a conjugate addition reactions between a nucleophile, e.g. an amine or a hydroxyl group, and an electrophile, e.g. a carboxylic acid or an aldehyde. In some embodiments, a nucleophile may exist on a linker and an electrophile may exist on a muscle-targeting agent or molecular payload prior to a reaction between a linker and a muscle-targeting agent or molecular payload. In some embodiments, an electrophile may exist on a linker and a nucleophile may exist on a muscle-targeting agent or molecular payload prior to a reaction between a linker and a muscle-targeting agent or molecular payload. In some embodiments, an electrophile may be an azide, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, or a thiol group.

D. Examples of Antibody-Molecular Payload Complexes

Other aspects of the present disclosure provide complexes comprising any one the muscle targeting agent (e.g., a transferrin receptor antibodies) described herein covalently linked to any of the molecular payloads (e.g., an oligonucleotide) described herein. In some embodiments, the muscle targeting agent (e.g., a transferrin receptor antibody) is covalently linked to a molecular payload (e.g., an oligonucleotide) via a linker. Any of the linkers described herein may be used. In some embodiments, the linker is linked to the 5′ end, the 3′ end, or internally of the oligonucleotide. In some embodiments, the linker is linked to the antibody via a thiol-reactive linkage (e.g., via a cysteine in the antibody).

An exemplary structure of a complex comprising a transferrin receptor antibody covalently linked to an oligonucleotide via a Val-cit linker is provided below:

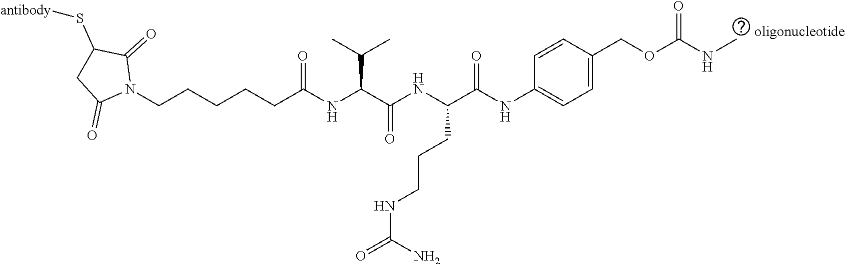

wherein the linker is linked to the 5′ end, the 3′ end, or internally of the oligonucleotide, and wherein the linker is linked to the antibody via a thiol-reactive linkage (e.g., via a cysteine in the antibody).

In some embodiments, a complex comprising a transferrin receptor antibody covalently linked to an oligonucleotide via a Val-cit linker is provided below:

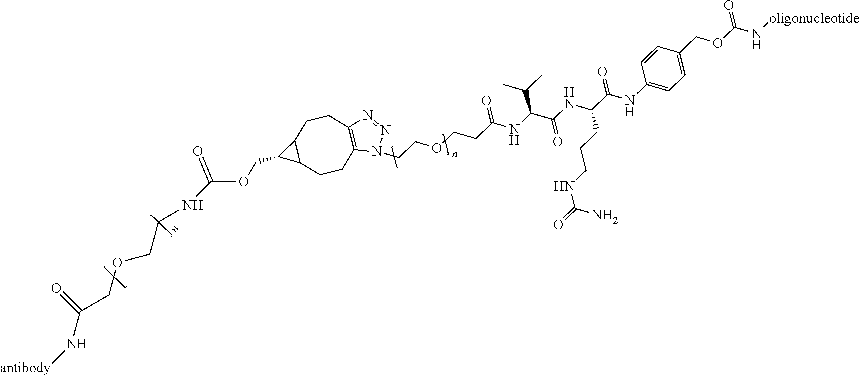

wherein the linker is linked to the 5′ end, the 3′ end, or internally of the oligonucleotide, and wherein the linker is linked to the antibody via a amine-reactive linkage (e.g., via a lysine in the antibody).

It should be appreciated that antibodies can be linked to oligonucleotides with different stochiometries, a property that may be referred to as a drug to antibody ratios (DAR) with the “drug” being the oligonucleotide. In some embodiments, one oligonucleotide is linked to an antibody (DAR=1). In some embodiments, two oligonucleotides are linked to an antibody (DAR=2). In some embodiments, three oligonucleotides are linked to an antibody (DAR=3). In some embodiments, four oligonucleotides are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. DAR may be increased by conjugating oligonucleotides to different sites on an antibody and/or by conjugating multimers to one or more sites on antibody. For example, a DAR of 2 may be achieved by conjugating a single oligonucleotide to two different sites on an antibody or by conjugating a dimer oligonucleotide to a single site of an antibody.

In some embodiments, the complex described herein comprises a transferrin receptor antibody (e.g., an antibody or any variant thereof as described herein) covalently linked to an oligonucleotide. In some embodiments, the complex described herein comprises a transferrin receptor antibody (e.g., an antibody or any variant thereof as described herein) covalently linked to an oligonucleotide via a linker (e.g., a Val-cit linker). In some embodiments, the linker (e.g., a Val-cit linker) is linked to the 5′ end, the 3′ end, or internally of the oligonucleotide. In some embodiments, the linker (e.g., a Val-cit linker) is linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide, wherein the transferrin receptor antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3.

In some embodiments, the complex described herein comprises a transferrin receptor antibody covalently linked to an oligonucleotide, wherein the transferrin receptor antibody comprises a VH having the amino acid sequence of SEQ ID NO: 33 and a VL having the amino acid sequence of SEQ ID NO: 34. In some embodiments, the complex described herein comprises a transferrin receptor antibody covalently linked to an oligonucleotide, wherein the transferrin receptor antibody comprises a VH having the amino acid sequence of SEQ ID NO: 35 and a VL having the amino acid sequence of SEQ ID NO: 36.

In some embodiments, the complex described herein comprises a transferrin receptor antibody covalently linked to an oligonucleotide, wherein the transferrin receptor antibody comprises a heavy chain having the amino acid sequence of SEQ ID NO: 39 and a light chain having the amino acid sequence of SEQ ID NO: 40. In some embodiments, the complex described herein comprises a transferrin receptor antibody covalently linked to an oligonucleotide, wherein the transferrin receptor antibody comprises a heavy chain having the amino acid sequence of SEQ ID NO: 41 and a light chain having the amino acid sequence of SEQ ID NO: 42.

In some embodiments, the complex described herein comprises a transferrin receptor antibody covalently linked to an oligonucleotide via a linker (e.g., a Val-cit linker), wherein the transferrin receptor antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3.

In some embodiments, the complex described herein comprises a transferrin receptor antibody covalently linked to an oligonucleotide via a linker (e.g., a Val-cit linker), wherein the transferrin receptor antibody comprises a VH having the amino acid sequence of SEQ ID NO: 33 and a VL having the amino acid sequence of SEQ ID NO: 34. In some embodiments, the complex described herein comprises a transferrin receptor antibody covalently linked to an oligonucleotide via a linker (e.g., a Val-cit linker), wherein the transferrin receptor antibody comprises a VH having the amino acid sequence of SEQ ID NO: 35 and a VL having the amino acid sequence of SEQ ID NO: 36.

In some embodiments, the complex described herein comprises a transferrin receptor antibody covalently linked to an oligonucleotide via a linker (e.g., a Val-cit linker), wherein the transferrin receptor antibody comprises a heavy chain having the amino acid sequence of SEQ ID NO: 39 and a light chain having the amino acid sequence of SEQ ID NO: 40. In some embodiments, the complex described herein comprises a transferrin receptor antibody covalently linked to an oligonucleotide via a linker (e.g., a Val-cit linker), wherein the transferrin receptor antibody comprises a heavy chain having the amino acid sequence of SEQ ID NO: 41 and a light chain having the amino acid sequence of SEQ ID NO: 42.

In some embodiments, the complex described herein comprises a transferrin receptor antibody covalently linked to an oligonucleotide via a Val-cit linker, wherein the transferrin receptor antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3, and wherein the complex comprises the structure of:

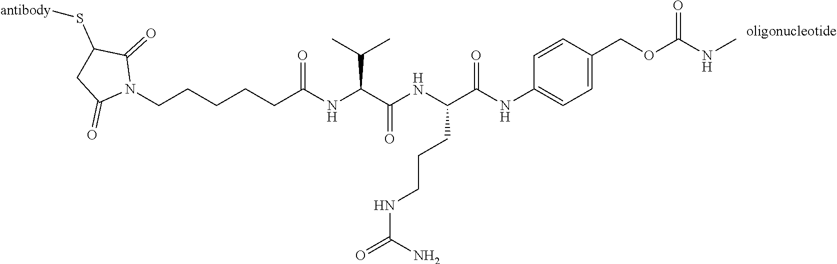

wherein the linker Val-cit linker is linked to the 5′ end, the 3′ end, or internally of the oligonucleotide, and wherein the Val-cit linker is linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide via a Val-cit linker, wherein the transferrin receptor antibody comprises a VH having the amino acid sequence of SEQ ID NO: 33 and a VL having the amino acid sequence of SEQ ID NO: 34, and wherein the complex comprises the structure of:

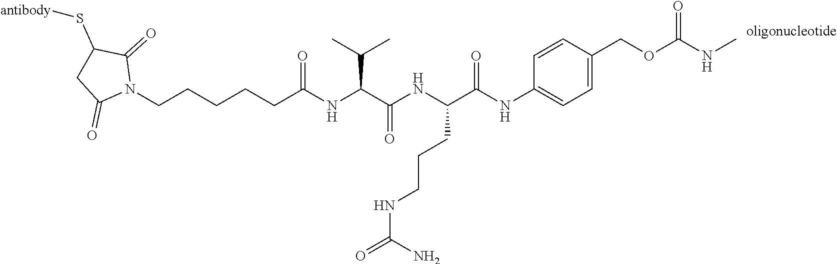

wherein the linker Val-cit linker is linked to the 5′ end, the 3′ end, or internally of the oligonucleotide, and wherein the Val-cit linker is linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide via a Val-cit linker, wherein the transferrin receptor antibody comprises a VH having the amino acid sequence of SEQ ID NO: 35 and a VL having the amino acid sequence of SEQ ID NO: 36, and wherein the complex comprises the structure of:

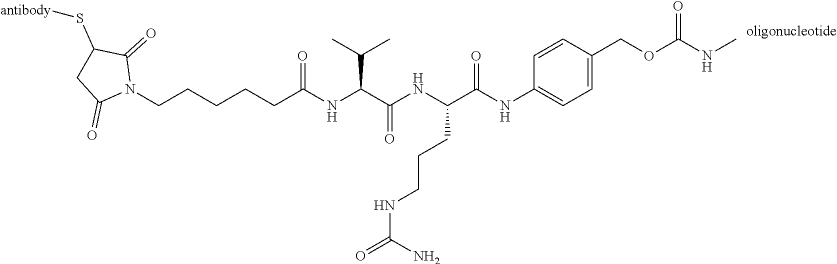

wherein the linker Val-cit linker is linked to the 5′ end, the 3′ end, or internally of the oligonucleotide, and wherein the Val-cit linker is linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide via a Val-cit linker, wherein the transferrin receptor antibody comprises a heavy chain having the amino acid sequence of SEQ ID NO: 39 and a light chain having the amino acid sequence of SEQ ID NO: 40, and wherein the complex comprises the structure of:

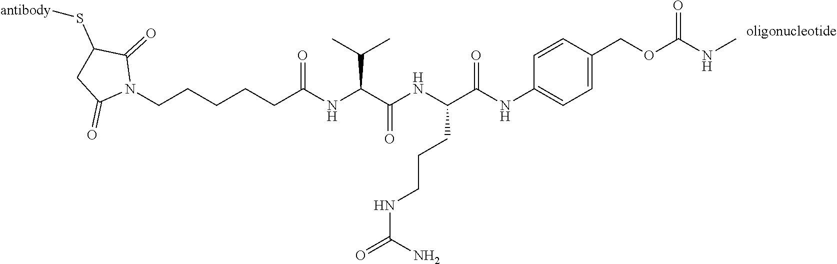

wherein the linker Val-cit linker is linked to the 5′ end, the 3′ end, or internally of an oligonucleotide, and wherein the Val-cit linker is linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide via a Val-cit linker, wherein the transferrin receptor antibody comprises a heavy chain having the amino acid sequence of SEQ ID NO: 41 and a light chain having the amino acid sequence of SEQ ID NO: 42, and wherein the complex comprises the structure of:

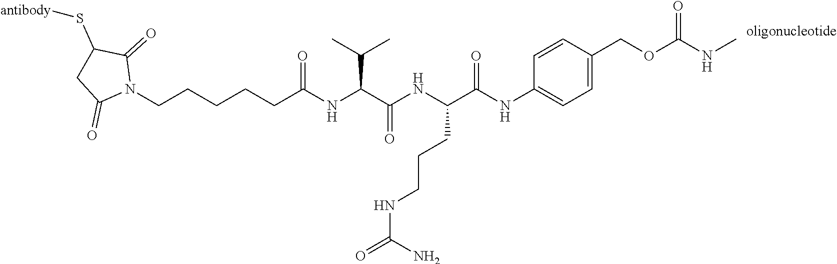

wherein the linker Val-cit linker is linked to the 5′ end, the 3′ end, or internally of an oligonucleotide, and wherein the Val-cit linker is linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to an oligonucleotide via a Val-cit linker, wherein the transferrin receptor antibody comprises a CDR-H1, a CDR-H2, and a CDR-H3 that are the same as the CDR-H1, CDR-H2, and CDR-H3 shown in Table 3; and a CDR-L1, a CDR-L2, and a CDR-L3 that are the same as the CDR-L1, CDR-L2, and CDR-L3 shown in Table 3, and wherein the complex comprises the structure of:

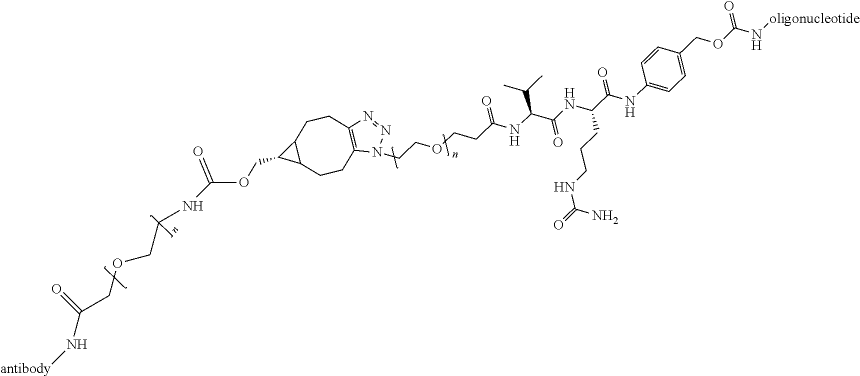

wherein the linker is linked to the 5′ end, the 3′ end, or internally of the oligonucleotide, and wherein the linker is linked to the antibody via a amine-reactive linkage (e.g., via a lysine in the antibody).

In some embodiments, the complex described herein comprises a transferrin receptor antibody covalently linked to an oligonucleotide via a Val-cit linker, wherein the transferrin receptor antibody comprises a VH having the amino acid sequence of SEQ ID NO: 33 and a VL having the amino acid sequence of SEQ ID NO: 34, and wherein the complex comprises the structure of:

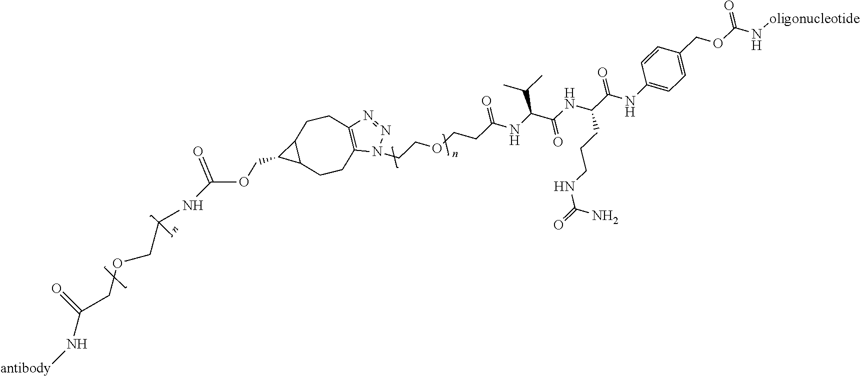

wherein the linker is linked to the 5′ end, the 3′ end, or internally of the oligonucleotide, and wherein the linker is linked to the antibody via a amine-reactive linkage (e.g., via a lysine in the antibody).

In some embodiments, the complex described herein comprises a transferrin receptor antibody covalently linked to an oligonucleotide via a Val-cit linker, wherein the transferrin receptor antibody comprises a VH having the amino acid sequence of SEQ ID NO: 35 and a VL having the amino acid sequence of SEQ ID NO: 36, and wherein the complex comprises the structure of:

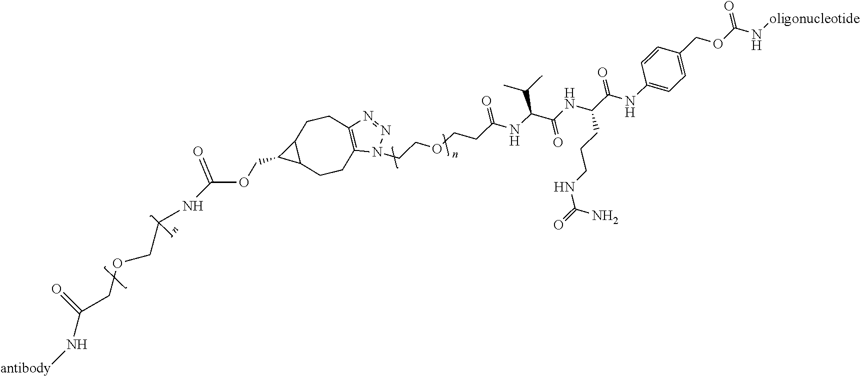

wherein the linker is linked to the 5′ end, the 3′ end, or internally of the oligonucleotide, and wherein the linker is linked to the antibody via a amine-reactive linkage (e.g., via a lysine in the antibody).

In some embodiments, the complex described herein comprises a transferrin receptor antibody covalently linked to an oligonucleotide via a Val-cit linker, wherein the transferrin receptor antibody comprises a heavy chain having the amino acid sequence of SEQ ID NO: 39 and a light chain having the amino acid sequence of SEQ ID NO: 40, and wherein the complex comprises the structure of:

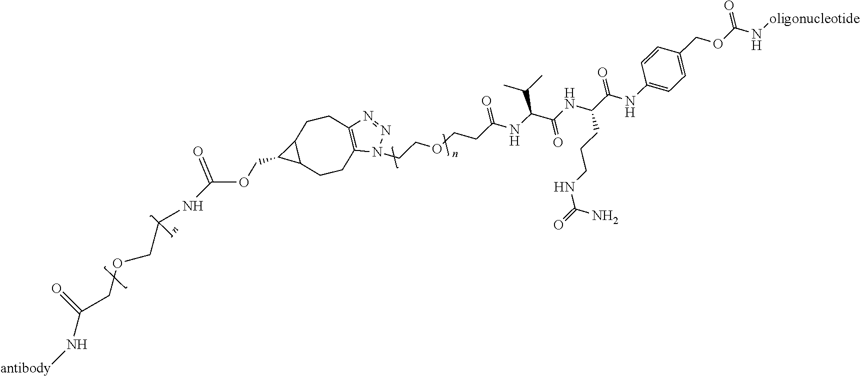

wherein the linker is linked to the 5′ end, the 3′ end, or internally of the oligonucleotide, and wherein the linker is linked to the antibody via a amine-reactive linkage (e.g., via a lysine in the antibody).

In some embodiments, the complex described herein comprises a transferrin receptor antibody covalently linked to an oligonucleotide via a Val-cit linker, wherein the transferrin receptor antibody comprises a heavy chain having the amino acid sequence of SEQ ID NO: 41 and a light chain having the amino acid sequence of SEQ ID NO: 42, and wherein the complex comprises the structure of:

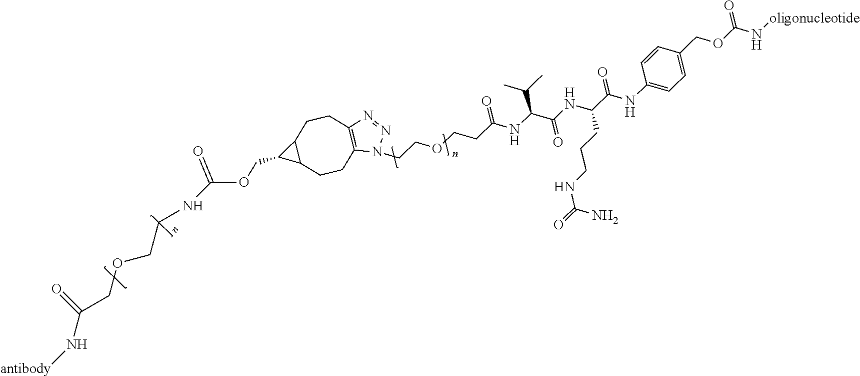

wherein the linker is linked to the 5′ end, the 3′ end, or internally of the oligonucleotide, and wherein the linker is linked to the antibody via a amine-reactive linkage (e.g., via a lysine in the antibody).

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous. In some embodiments, the route of administration is extramuscular parenteral administration.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the a complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently to a molecular payload as described herein are effective in treating a muscle disease (e.g., a rare muscle disease). In some embodiments, complexes are effective in treating a muscle disease provided in Table 1. In some embodiments, a muscle disease is associated with a disease allele, for example, a disease allele for a particular muscle disease may comprise a genetic alteration in a corresponding gene listed in Table 1.

In some embodiments, a subject may be a human subject, a non-human primate subject, a rodent subject, or any suitable mammalian subject. In some embodiments, a subject may have a muscle disease provided in Table 1.

An aspect of the disclosure includes a methods involving administering to a subject an effective amount of a complex as described herein. In some embodiments, an effective amount of a pharmaceutical composition that comprises a complex comprising a muscle-targeting agent covalently to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently to a molecular payload is administered at an effective concentration that confers therapeutic effect on a subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex in a subject, generally will contribute to determination of the concentration of pharmaceutical composition that is used for treatment. The frequency of administration may be empirically determined and adjusted to maximize the efficacy of the treatment.

Generally, for administration of any of the complexes described herein, an initial candidate dosage may be about 1 to 100 mg/kg, or more, depending on the factors described above, e.g. safety or efficacy. In some embodiments, a treatment will be administered once. In some embodiments, a treatment will be administered daily, biweekly, weekly, bimonthly, monthly, or at any time interval that provide maximum efficacy while minimizing safety risks to the subject. Generally, the efficacy and the treatment and safety risks may be monitored throughout the course of treatment

The efficacy of treatment may be assessed using any suitable methods. In some embodiments, the efficacy of treatment may be assessed by evaluation of observation of symptoms associated with a muscle disease.

In some embodiments, a pharmaceutical composition that comprises a complex comprising a muscle-targeting agent covalently to a molecular payload described herein is administered to a subject at an effective concentration sufficient to inhibit activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

In some embodiments, a pharmaceutical composition may comprises more than one complex comprising a muscle-targeting agent covalently to a molecular payload. In some embodiments, a pharmaceutical composition may further comprise any other suitable therapeutic agent for treatment of a subject, e.g. a human subject having a muscle disease (e.g., a muscle disease provided in Table 1). In some embodiments, the other therapeutic agents may enhance or supplement the effectiveness of the complexes described herein. In some embodiments, the other therapeutic agents may function to treat a different symptom or disease than the complexes described herein.

### EXAMPLES

### Example 1: Synthesis of a Complex Comprising an Antibody Linked to an Oligonucleotide

A muscle-targeting complex was generated comprising an antisense oligonucleotide that targets DMPK (‘DMPK ASO’, also known as DTX-P-060) covalently linked, via a cathepsin cleavable linker, to an anti-transferrin receptor hIgG1-kappa antibody (DTX-A-001).

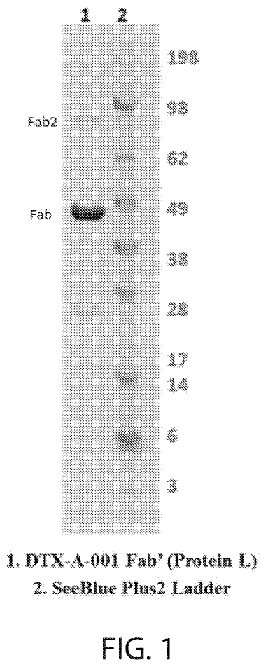

DTX-A-001 was stably expressed in CHO-K1SP cells (Genscript) in a 15L batch-fed culture that produced 4.6 g of protein-A purified DTX-A-001. The purified DTX-A-001 was site specifically cleaved (at sequence of CPAPELLG-GPSVF) and purified to F(ab′)2 fragments using FragIt solid supported IdeS enzyme and anti-FC affinity resin (Genovis #A2-FR2-1000), according to manufacturer specifications. The F(ab′)2 fragments were reduced to Fab′ with 80-molar excess of cysteamine-HCl (ChemImpex #02839) per hinge thiol at 37° C. for 90 min. Fab′ was then immediately purified away from non-reduced F(ab′)2 and free cysteamine with protein L chromatography (GE #17547855, 10X series) using a standard pH gradient (50 mM Na₃C6H₅O₇, pH=2.6, 60-100% gradient over 20 column volumes). As shown in FIG. 1, DTX-A-001 Fab′ (˜49 kDa) was substantially pure following protein L chromatography.

Eluted DTX-A-001 Fab′ was neutralized with 1.0M NaOH to pH=7.2, diluted by 1:10 v:v with acetonitrile, and reacted with a 10-molar excess of BCN-PEG₃-Mal (Bicyclononyne-PEG₃-Maleimide (endo)) overnight at 25° C. Fab′ that incorporated BCN (1.3-1.6 moles of BCN per mole of Fab′) was isolated and carried on to the next step.

A DMPK ASO compound comprising DTX-P-060 and an azide-Valine-Citrulline linker was generated. DTX-P-60, dissolved at 200 mg/mL in RNAse free water, was diluted to 10 mg/mL with dry dimethylformamide. A 25-fold molar excess of tributylamine was then added. Linker (Azide-PEG3-Val-Cit-PAB-PNP, dissolved at 20 mg/mL in DMF) was finally added at a 2-fold molar excess to the DTX-P-60 solution for 120 min at −25° C. Reaction completion measured using ninhydrin (Kaiser test) prior to quenching the reaction using an alcohol precipitation. The alcohol precipitation was accomplished by addition of 0.1 v/v 3 M NaCl solution, followed by addition of 3 volumes of −80° C. isopropanol. The solution was then thoroughly mixed and subsequently allowed to precipitate at −20° C. for 1 hour. The precipitated solution was centrifuged (at 4300×g; 8° C.) for 30 mins and the solvent was decanted. The pellet was washed with −80° C. 80% Ethanol in RNase free water (one volume equivalent to starting reaction) and centrifuged (at 4300×g; 8° C.) for 20 min. Ethanol was decanted and the pellet (containing the DMPK ASO compound comprising DTX-P-060 and an azide-Valine-Citrulline linker) was dried for 10 min at 37° C. The DMPK ASO compound comprising DTX-P-060 and an azide-Valine-Citrulline linker was resuspended in 20% Acetonitrile in Nuclease free water (at concentration of 20 mg/mL).

DTX-A-001 Fab′ comprising incorporated BCN was reacted with 1.2 molar equivalents per BCN of the DMPK ASO compound comprising DTX-P-060 and an azide-Valine-Citrulline linker. The coupling reaction proceeded for 2 hours at 25° C. followed by overnight at 4° C.

The completion of the coupling reaction was evaluated by SDS-PAGE, demonstrating a 72% coupling efficiency by densitometry (72% of total antibody linked to at least one DMPK ASO). Of the antibody that was linked to at least one DMPK ASO, ˜70% of the DTX-A-001 Fab′ was linked to one DMPK ASO (Drug-to-antibody ratio (DAR) of 1), ˜20% of the DTX-A-001 Fab′ was linked to two DMPK ASOs (DAR of 2), and ˜5% of the DTX-A-001 Fab′ was linked to three or more DMPK ASO (DAR of 3+).

### Example 2: Synthesis of a Complex Comprising an Antibody Linked to an Oligonucleotide

A muscle-targeting complex was generated comprising an antisense oligonucleotide that targets DMPK (‘DMPK ASO’, also known as DTX-P-060) covalently linked, via a cathepsin cleavable linker, to an anti-transferrin receptor hIgG1-kappa antibody (DTX-A-001).

DTX-A-001 was stably expressed in CHO-K1SP cells (Genscript) in a 15L batch-fed culture that produced protein-A purified DTX-A-001. The purified DTX-A-001 was site specifically cleaved (at sequence of CPAPELLG-GPSVF) and purified to F(ab′)2 fragments using FragIt solid supported IdeS enzyme and anti-FC affinity resin (Genovis #A2-FR2-1000), according to manufacturer specifications. FC domain and any uncut IgG was removed using CapturSelect FcXL (ThermoScientific) with binding capacity of 25 mg/mL on HiLoad column (26 mm×40 cm, [Load flow rate: linear 113 cm/hr with 26 mm ID-10.0 ml/min volumetric flow, residence time-21.2 minutes]). The F(ab′)2 fragments were reduced to Fab′ with 80-molar excess of cysteamine-HCl (ChemImpex #02839) per hinge thiol at 37° C. for 90 min. Fab′ was then immediately purified away from non-reduced F(ab′)2 and free cysteamine with protein L chromatography (GE #17547855, 10X series) using a standard pH gradient (50 mM Na₃C₆H₅O₇, pH=2.6, 60-100% gradient over 20 column volumes). As shown in FIG. 1, DTX-A-001 Fab′(˜49 kDa) was substantially pure following protein L chromatography.

Eluted DTX-A-001 Fab′ was neutralized with 1.0 M NaOH to pH=7.2, diluted by 1:10 v:v with acetonitrile, and reacted with a 5-molar excess of BCN-PEG₄-PFP (Bicyclononyne-PEG4-pentafluorophenyl (endo)) overnight at 25° C. Fab′ that incorporated BCN (1.3-1.6 moles of BCN per mole of Fab′) was isolated and carried on to the next step.

A DMPK ASO compound comprising DTX-P-060 and an azide-Valine-Citrulline linker was generated. DTX-P-60, dissolved at 200 mg/mL in RNAse free water, was diluted to 10 mg/mL with dry dimethylformamide. A 25-fold molar excess of tributylamine was then added. Linker (Azide-PEG3-Val-Cit-PAB-PNP, dissolved at 20 mg/mL in DMF) was finally added at a 2-fold molar excess to the DTX-P-60 solution for 120 min at −25° C. Reaction completion measured using ninhydrin (Kaiser test) prior to quenching the reaction using an alcohol precipitation. The alcohol precipitation was accomplished by addition of 0.1 v/v 3 M NaCl solution, followed by addition of 3 volumes of −80° C. isopropanol. The solution was then thoroughly mixed and subsequently allowed to precipitate at −20° C. for 1 hour. The precipitated solution was centrifuged (at 4300×g; 8° C.) for 30 mins and the solvent was decanted. The pellet was washed with −80° C. 80% Ethanol in RNase free water (one volume equivalent to starting reaction) and centrifuged (at 4300×g; 8° C.) for 20 min. Ethanol was decanted and the pellet (containing the DMPK ASO compound comprising DTX-P-060 and an azide-Valine-Citrulline linker) was dried for 10 min at 37° C. The DMPK ASO compound comprising DTX-P-060 and an azide-Valine-Citrulline linker was resuspended in 20% Acetonitrile in Nuclease free water (at concentration of 20 mg/mL).

DTX-A-001 Fab′ comprising incorporated BCN was reacted with 1.2 molar equivalents per BCN of the DMPK ASO compound comprising DTX-P-060 and an azide-Valine-Citrulline linker. The coupling reaction proceeded for 2 hours at 25° C. followed by overnight at 4° C.

The completion of the coupling reaction was evaluated by SDS-PAGE.

### Example 3: Successful Purification of Complexes Comprising Muscle-Targeting Antibodies Linked to Oligonucleotides

The crude mixture of a complex comprising DTX-A-001 Fab′ linked to DMPK ASO, unlinked DMPK ASO, and unlinked DTX-A-001 Fab′ from Example 1 was successfully purified using sequential chromatography steps. Unlinked DTX-A-001 Fab′ was first removed using a hydrophobic interaction chromatography resin followed by removal of unlinked DMPK ASO using a ceramic hydroxyapatite resin.

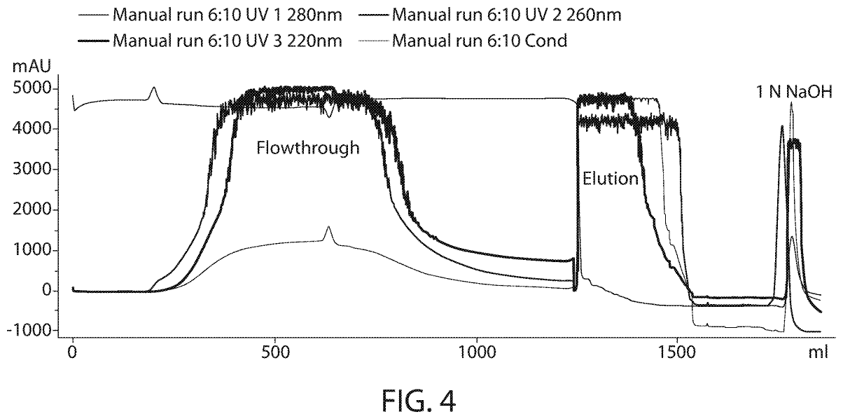

Unlinked Fab′ (28% of total protein) was removed using a HIC resin (HiTrap Butyl HP, from GE Healthcare Life Sciences; GE #28411005). The 5 mL HIC column was equilibrated with 5 column volumes (CV) of 600 mM ammonium sulfate in nuclease free water, pH=7.0. The unpurified mixture of DTX-A-001 Fab′ linked to DMPK ASO, unlinked DMPK ASO, and unlinked DTX-A-001 Fab′ was diluted with 2M stock concentrations of ammonium sulfate to achieve a final ammonium sulfate concentration of 600 mM. The unpurified mixture was loaded to the HIC column at a flow rate of 3-10 mL/min. Unbound material, such as unlinked DTX-A-001 Fab′ was washed with 3-5 CV of 600 mM ammonium sulfate. Following removal of the unlinked DTX-A-001 Fab′, the DTX-A-001 Fab′ linked to DMPK ASO and unlinked DMPK ASO were eluted from the HIC column with 2 CV of an eluent solution (5-20 mM Na₂HPO₄, 25 mM NaCl, pH 7.0) into 1 CV fractions (FIG. 4).

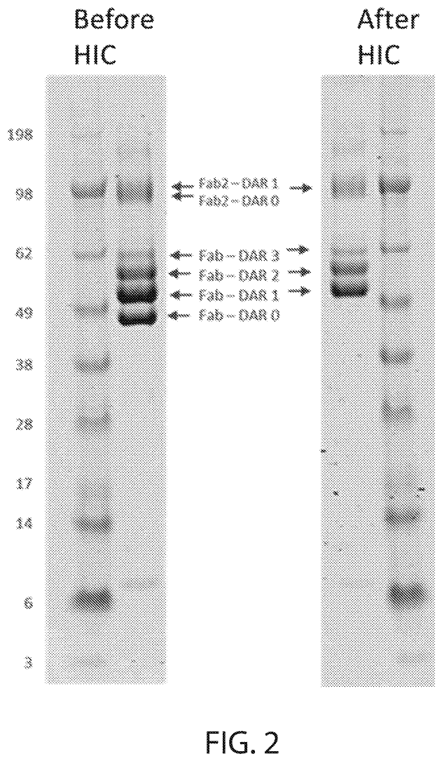

The isolated mixture of DTX-A-001 Fab′ linked to DMPK ASO and unlinked DMPK ASO were analyzed on SDS-PAGE and analytical SEC to ensure sufficient (e.g., complete) removal of unlinked Fab′ (FIG. 2). The HIC purification step successfully removed all of the Fab2-DAR 0 and Fab-DAR 0 (DAR=drug-to-antibody ratio). Note that Fab2 is a dimer of DTX-A-001 Fab′.

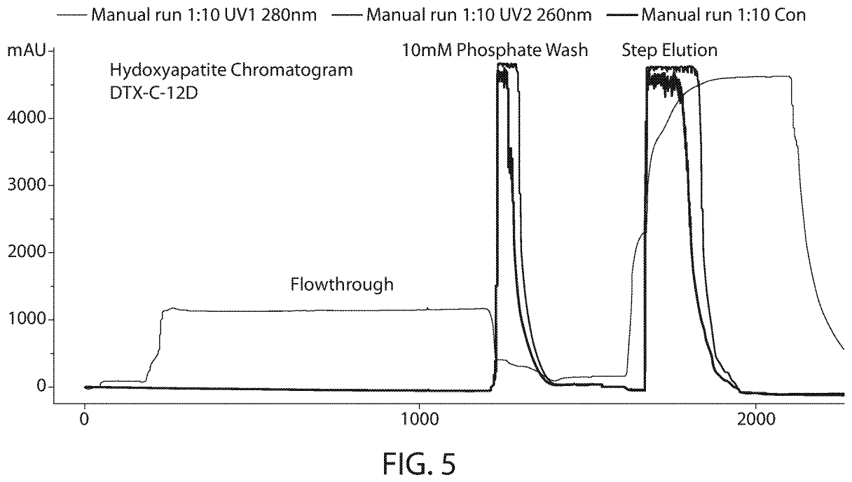

The isolated mixture of DTX-A-001 Fab′ linked to DMPK ASO and unlinked DMPK ASO was then diluted 1:3 in nuclease free water and loaded onto, e.g., contacted with, a ceramic hydroxyapatite (HA) column (Bio-Scale™ Mini CHT™ 40 μm, from Biorad; Catalog #732-4324) at a biomolecule concentration of 8 mg/mL of resin. The HA column was washed with 5 CV of a wash solution (5 mM Na₂HPO₄, 25 mM NaCl pH 7.0) to remove unlinked DMPK ASO. Following removal of the unlinked DMPK ASO, the complex comprising DTX-A-001 Fab′ linked to DMPK ASO was eluted from the HA column with 3 CV of an eluent solution (100 mM Na₂HPO₄, 100 mM NaCl, pH 7.6) (FIG. 5).

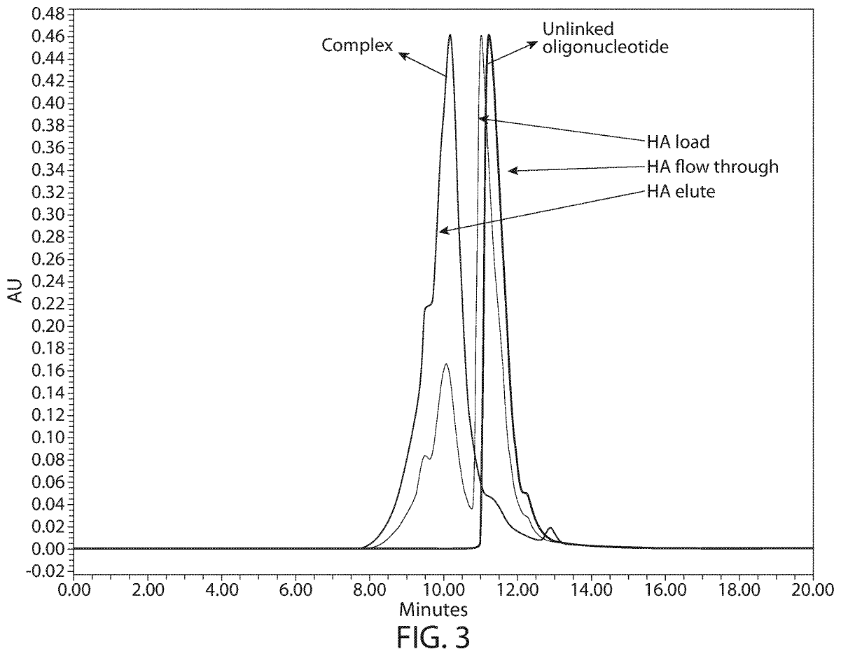

Isolated and purified DTX-A-001 Fab′ linked to DMPK ASO was analyzed on SDS-PAGE and analytical SEC to demonstrate complete removal of unlinked DMPK ASO. As shown in the overlaid analytical SEC chromatograms in FIG. 3, the elution fraction from the HA resin provided substantially purified complex comprising DTX-A-001 Fab′ linked to DMPK ASO. Further, the HA flow-through (e.g., the wash fraction) comprises purified DMPK ASO. These data demonstrate that the complex can be purified from the unlinked oligonucleotide using hydroxyapatite resin, a surprising purification result that was otherwise unattainable.

Several alternative strategies for the purification of the crude mixture of a complex comprising DTX-A-001 Fab′ linked to DMPK ASO, unlinked DMPK ASO, and unlinked DTX-A-001 Fab′ from Example 1 were examined, including cation exchange (CEX) and anion exchange (AEX) resins. It was found that none of the alternative strategies was as effective as the mixed mode resin approach (using ceramic hydroxyapatite resin).

### Example 4: Purification of Fab-Oligonucleotide Complexes Using Hydrophobic Interaction Chromatography

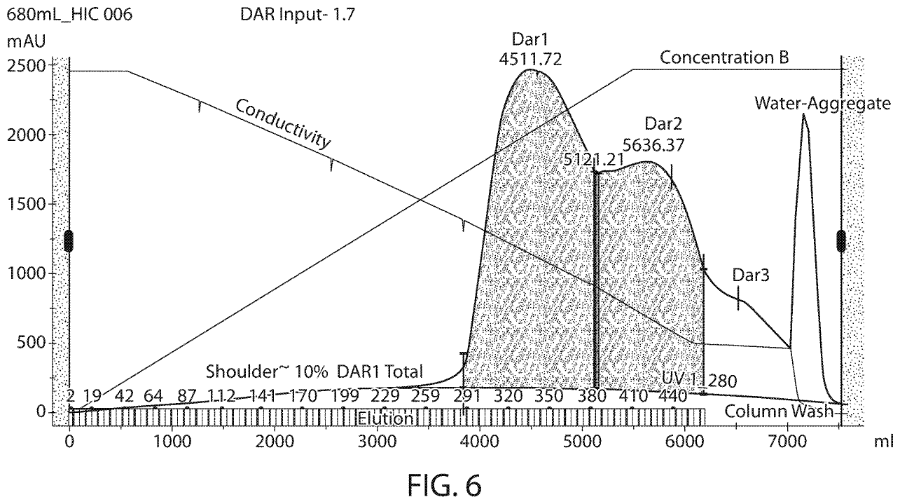

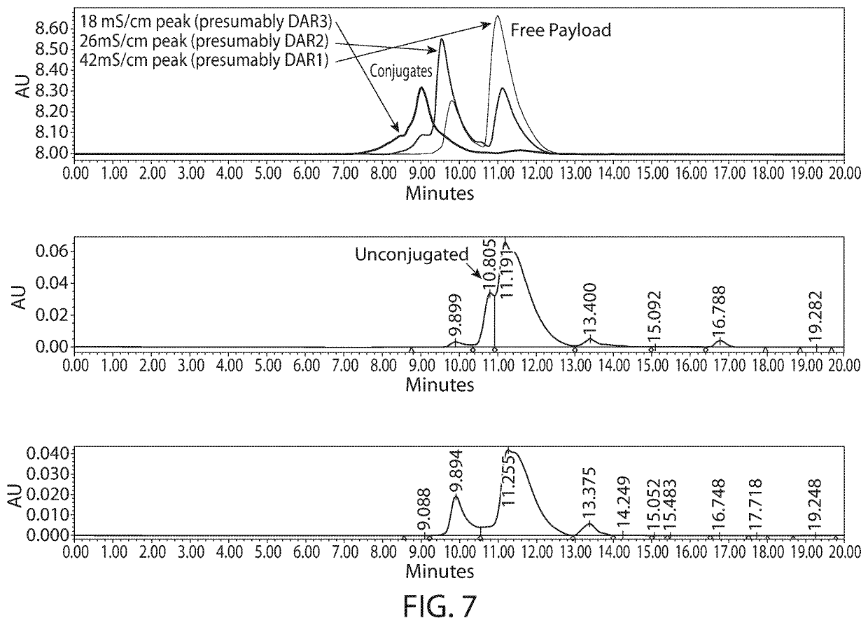

In addition to removing unlinked DTX-A-001 Fab′ using HIC as described in Example 3, HIC can also be used for separating DAR species by using an ammonium sulfate gradient for elution. After equilibrating the HIC column with 3 CV's of 600 mM ammonium sulfate at pH 7.0, a mixture of conjugates (DAR1, DAR2, and DAR3), unlinked Fab′, and 600 mM ammonium sulfate was loaded onto a 668 ml column (50 mm×34 cm) packed with capto butyl resin (Cytiva #17545903). For the equilibration, wash, and gradient steps, a linear flow rate of 76 cm/hr (25 ml/min) was used. As described above, the unlinked Fab′ was removed in the flowthrough during the loading of the column. The column was washed with 3 CV's of 600 mM ammonium sulfate, pH 7.0 to completely wash the unbound, unlinked Fab′. Upon completion of the wash, a linear gradient was performed from 600 mM ammonium sulfate, pH 7.0 to 100 mM ammonium sulfate, 10 mM Sodium phosphate, 25 mM NaCl, pH6.5 for H 7.0 over 8 CVs. A pure DAR1 species eluted first with a peak centered at 41 mS/cm. A second peak centered at 33 mS/cm eluted next, containing a mostly DAR2. A third shoulder peak eluted at 20 mS/cm, containing mostly DAR 3 (FIG. 6 and FIG. 7).

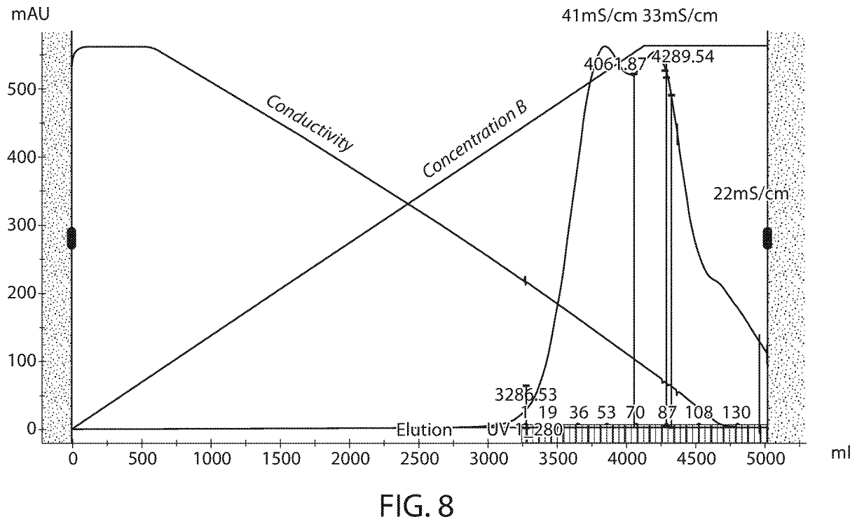

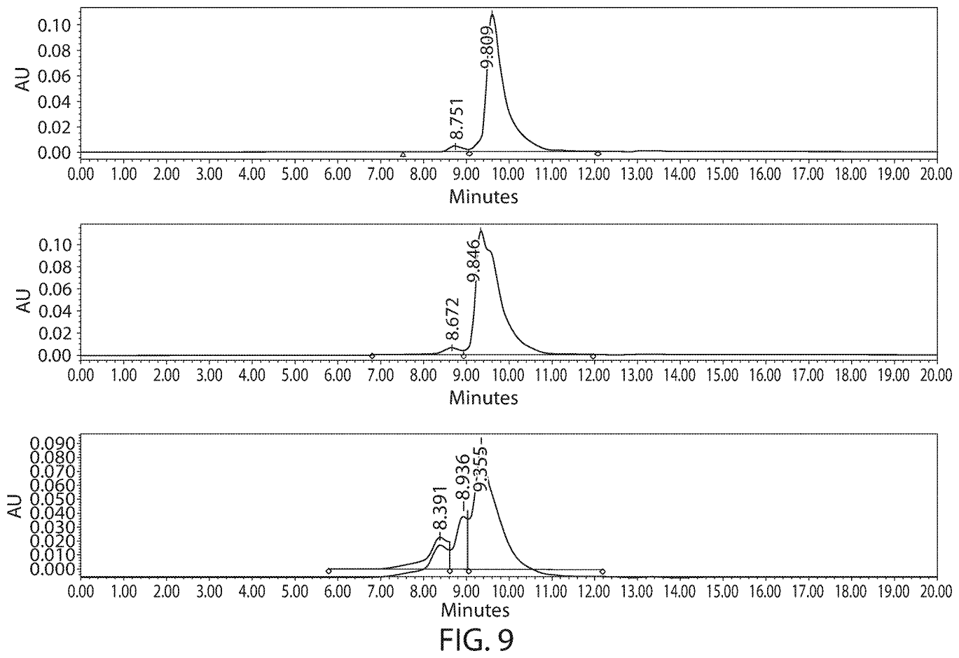

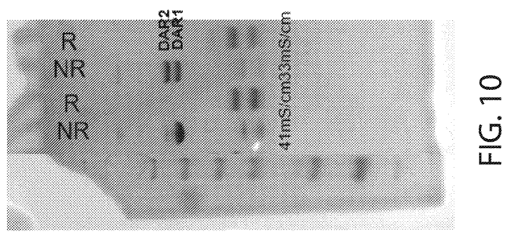

Using the same conditions as described above, while instituting a steeper gradient (6 CVs instead of 8), resulted in less resolution of DAR 1 and DAR 2, but a similar pattern of DAR retention (FIGS. 8-10).

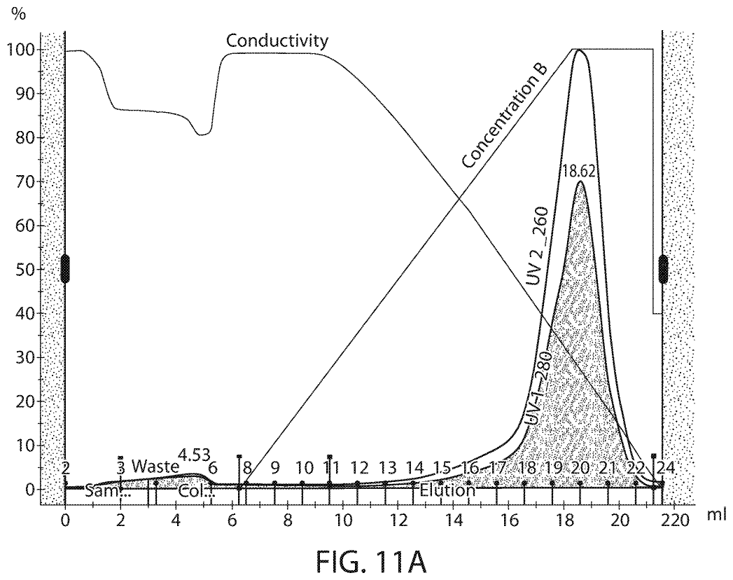

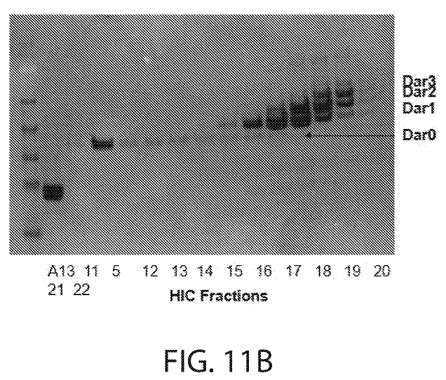

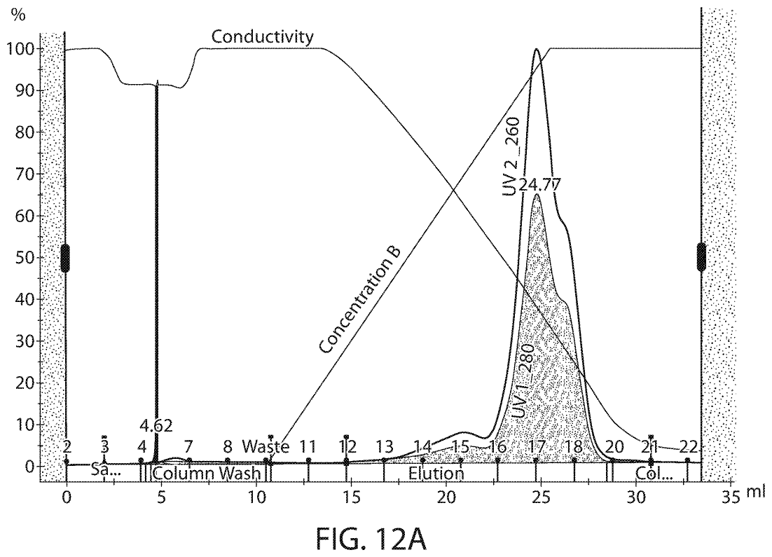

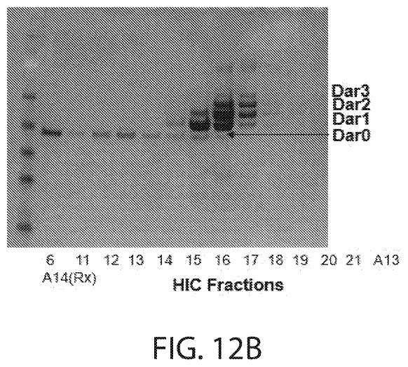

Further, the HIC linear gradient was used on a packed 1.5 ml capto butyl HIC column (small scale) for the purification of two additional conjugates with different Fab′ and oligonucleotide sequences. Briefly, the 1 ml column was equilibrated with 800 mM ammonium sulfate, pH 7.0 for 10 CVs using a linear flow rate of 156 cm/hr (1 ml/min). After equilibration, the column was loaded with a mixture, having a concentration of 800 mM ammonium sulfate, of DAR 1, DAR 2, and DAR 3 conjugates as well as unlinked Fab′ and unconjugated payload. A column wash at 800 mM ammonium sulfate was performed for 5 CVs. After the wash, a linear gradient from 800 mM Ammonium sulfate to 10 mM Sodium phosphate, 25 mM NaCl, pH6.5 was performed over 12 CVs, using a linear flow rate of 39 cm/hr (0.25 ml/min). The conjugates eluted over the gradient (FIG. 11A and FIG. 12A) and some degree of DAR species separation was achieved (FIG. 11B and FIG. 12B).

### Example 5: Purification of mAb-Oligonucleotide Complexes Using Hydrophobic Interaction Chromatography and Mixed-Mode Resin Chromatography

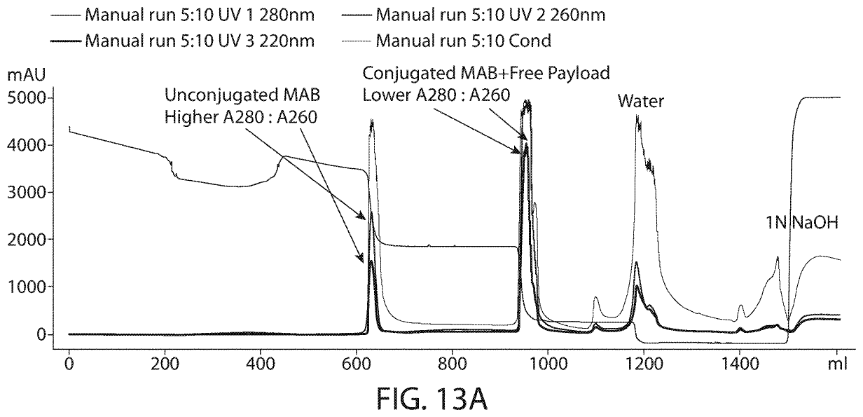

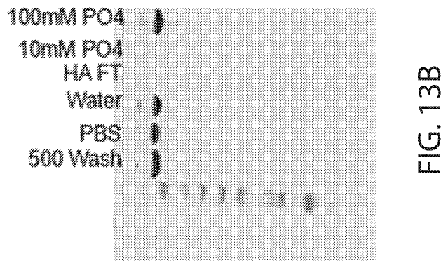

In addition to using the HIC to remove unlinked Fab′, the HIC was able to separate unlinked full length monoclonal antibodies (mAb) from conjugates. Using a 200 ml packed capto butyl column (26 mm×40 cm) and equilibrating at 1 M ammonium sulfate, a mixture conjugate and unconjugated mAbs as well as free oligonucleotides was loaded onto the column at a concentration of 800 mM ammonium sulfate, pH 7.0. A column wash step at 500 mM ammonium sulfate, pH 7.0 was performed in which the unlinked mAb was removed. The conjugates as well as free payload eluted at 20 mS/cm using a PBS buffer, pH 7.4 (FIGS. 13A and 13B).

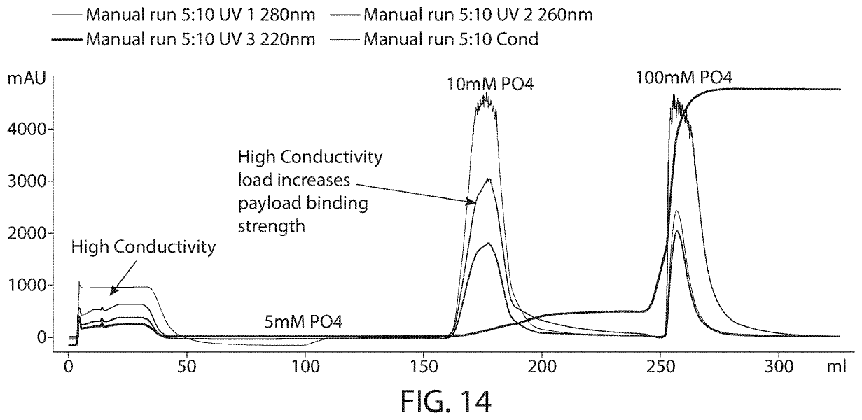

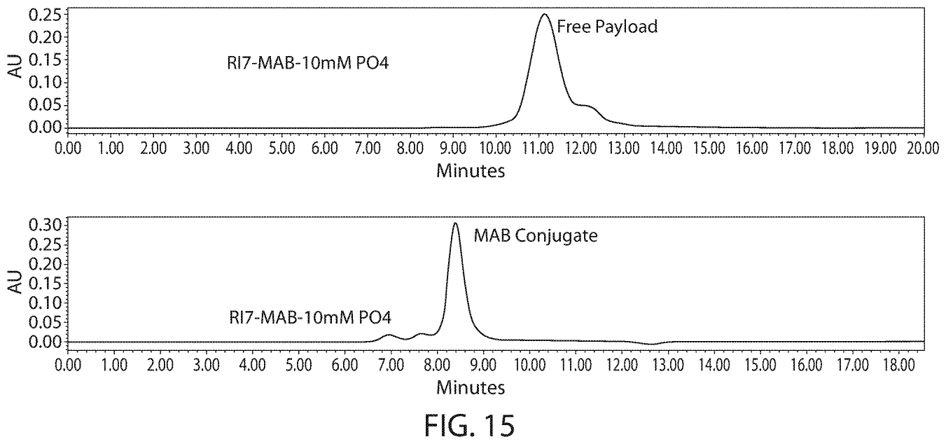

To remove free payload, a 50 ml mixed-mode fluorapatite (FA) column was used. The eluate from the HIC column was diluted 1:3 in nuclease free water. After equilibrating the column with 10 mM sodium phosphate, 10 mM sodium chloride, pH 7.6, the diluted HIC eluate was loaded onto the column. A wash step using 10 mM sodium phosphate, 10 mM sodium chloride, pH 7.6 was performed for 5 CVs. During this wash step, the free payload loaded onto the column eluted. After washing was complete, the mAB conjugates were eluted using a 100 mM sodium phosphate, 100 mM sodium chloride, pH 7.6 (FIGS. 14 and 15).

FA resin is more stable at pH below 6 than the HA column. In situations where a lower pH is needed for binding to the mixed-mode column, FA column may be used instead of the HA column. Similar performance of purification was observed for both FA and HA columns.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. A method of isolating a complex or plurality of complexes each comprising an antibody covalently linked to one or more oligonucleotides, the method comprising:
(i) contacting a mixture comprising the complexes and unlinked antibodies with a hydrophobic resin under conditions in which the complexes but not the unlinked antibodies adsorb to the hydrophobic resin, thus separating the unlinked antibodies from the complexes adsorbed to the hydrophobic resin; and
(ii) eluting the complexes from the hydrophobic resin under conditions in which the complex dissociate from the hydrophobic resin.

2. The method of claim 1, wherein the conditions in step (i) comprise a conductivity of at least 70 mS/cm, and/or the conditions in step (ii) comprises a conductivity of 10-70 mS/cm.

3. The method of claim 2, wherein the conditions in step (i) and or step (ii) are achieved using an anti-chaotropic salt, optionally wherein the anti-chaotropic salt is ammonium sulfate.

4. The method of any one of claims 1-3, wherein the mixture in step (i) further comprises at least 500 mM of ammonium sulfate, optionally wherein the mixture in step (i) further comprises 500 mM-1 M ammonium sulfate.

5. The method of any one of claims 1-4, further comprising washing the hydrophobic resin between step (i) and step (ii) with a solution comprising at least 500 mM of ammonium sulfate.

6. The method of any one of claims 1-5, wherein step (ii) comprises applying an elution solution comprising up to 200 mM of chloride ions and up to 100 mM of ammonium sulfate to the hydrophobic resin to elute the complexes.

7. The method of claim 6, wherein the elution solution does not contain ammonium sulfate.

8. The method of claim 6 or claim 7, wherein the elution solution is PBS.

9. The method of claim 6 or claim 7, wherein the elution solution comprises up to 25 mM chloride ions.

10. The method of any one of claims 1-5, wherein step (ii) comprises applying a gradually decreasing concentration of ammonium sulfate to the hydrophobic resin to elute the complexes, optionally wherein the concentration of ammonium sulfate decreases from at least 500 mM to less than 100 mM.

11. The method of claim 10, wherein the gradually decreasing concentration of ammonium sulfate is applied over 5-12 column volumes (CVs), optionally 6-8 CVs.

12. The method of any one of claims 1-11, wherein the mixture in step (i) further comprises unlinked oligonucleotides, optionally wherein the oligonucleotides adsorb to the hydrophobic resin in step (i) and are eluted in step (ii) with the complexes.

13. The method of any one of claims 1-12, wherein the antibody is a full length IgG, a Fab fragment, a Fab′ fragment, a F(ab′)2 fragment, a scFv, or a Fv fragment.

14. The method of any one of claims 1-13, wherein the antibody is an anti-transferrin receptor antibody.

15. The method of any one of claims 1-14, wherein the oligonucleotide is single stranded.

16. The method of claim 15, wherein the oligonucleotide is an antisense oligonucleotide, optionally a gapmer or a phosphorodiamidate morpholino oligomer (PMO).

17. The method of claim 15, wherein the oligonucleotide is one strand of a double stranded oligonucleotide, optionally wherein the double stranded oligonucleotide is a siRNA, and optionally wherein the one strand is the sense strand of the siRNA.

18. The method of any one of claims 1-17, wherein the oligonucleotide comprises at least one modified internucleotide linkage, optionally wherein the at least one modified internucleotide linkage is a phosphorothioate linkage.

19. The method of any one of claims 1-18, wherein the oligonucleotide comprises one or more modified nucleotides, optionally wherein the modified nucleotide comprises 2′-O-methoxyethylribose (MOE), locked nucleic acid (LNA), a 2′-fluoro modification, or a morpholino modification.

20. The method of any one of claims 1-19, wherein the oligonucleotide is 10-50 nucleotides in length, optionally 15-25 nucleotides in length.

21. The method of any one of claims 15-20, wherein the antibody is covalently linked to the 5′ of the oligonucleotide.

22. The method of any one of claims 15-20, wherein the antibody is covalently linked to the 3′ of the oligonucleotide.

23. The method of any one of claims 1-22, wherein the antibody is covalently linked to the oligonucleotide via a linker, optionally a Val-cit linker.

24. The method of any one of claims 1-23, wherein the complexes eluted in step (ii) comprises an antibody covalently linked to 1, 2, or 3 oligonucleotides.

25. The method of any one of claims 1-24, wherein the hydrophobic resin comprises a hydrophobic moiety selected butyl, t-butyl, phenyl, ether, amide, or propyl groups.

26. The method of any one of claims 1-25, wherein the hydrophobic resin is equilibrated prior to step (i), optionally equilibrated with a solution comprising at least 500 mM of ammonium sulfate.

27. The method of any one of claims 1-26, wherein the eluent obtained from step (ii) comprises undetectable levels of unlinked antibodies.

28. The method of any one of claims 12-27, further comprising isolating the complexes from the unlinked oligonucleotides.

29. A method of isolating a complex or plurality of complexes each comprising an antibody covalently linked to one or more oligonucleotides, the method comprising:
(i) contacting a mixture comprising the complexes and unlinked oligonucleotides with a mixed-mode resin that comprises positively-charged metal sites and negatively charged ionic sites, under conditions in which the complexes adsorb to the mixed-mode resin, and
(ii) eluting the complexes from the mixed-mode resin under conditions in which the complexes dissociate from the mixed-mode resin.

30. The method of claim 29, wherein the mixed-mode resin is an apatite resin.

31. The method of claim 30, wherein the apatite resin is a hydroxyapatite resin, a ceramic hydroxyapatite resin, a hydroxyfluoroapatite resin, a fluoroapatite resin, or a chlorapatite resin.

32. The method of any one of claims 29-31, wherein the mixture in step (i) further comprises up to 20 mM phosphate ions and/or up to 30 mM chloride ions, optionally wherein the mixture in step (i) further comprises up to 10 mM phosphate ions and/or up to 25 mM chloride ions.

33. The method of claim 32, wherein the unlinked oligonucleotide does not adsorb to the mixed-mode resin in step (i).

34. The method of any one of claims 29-31, wherein the mixture in step (i) further comprises up to 5 mM phosphate ions and/or up to 10 mM chloride ions, optionally wherein the mixture in step (i) further comprises up to 3 mM phosphate ions and/or up to 8 mM chloride ions.

35. The method of claim 34, wherein some or all of the unlinked oligonucleotide adsorb to the mixed-mode resin in step (i).

36. The method of claim 34 or claim 35, further comprising washing the mixed-mode resin between step (i) and step (ii) with a solution comprising up to 20 mM phosphate ions and/or up to 30 mM chloride ions, optionally wherein the solution comprises up to 10 mM phosphate ions and/or up to 25 mM chloride ions.

37. The method of any one of claims 29 to 36, wherein step (ii) comprises applying an elution solution comprising at least 30 mM phosphate ions and/or at least 50 mM chloride ions to the mixed-mode resin to elute the complexes, optionally wherein the elution solution comprises at least 100 mM phosphate ions and/or at least 100 mM chloride ions.

38. The method of any one of claims 29 to 37, wherein the antibody is a full length IgG, a Fab fragment, a Fab′ fragment, a F(ab′)2 fragment, a scFv, or a Fv fragment.

39. The method of any one of claims 29-38, wherein the antibody is an anti-transferrin receptor antibody.

40. The method of any one of claims 29-39, wherein the oligonucleotide is single stranded.

41. The method of claim 40, wherein the oligonucleotide is an antisense oligonucleotide, optionally a gapmer or a phosphorodiamidate morpholino oligomer (PMO).

42. The method of claim 41, wherein the oligonucleotide is one strand of a double stranded oligonucleotide, optionally wherein the double stranded oligonucleotide is a siRNA, and optionally wherein the one strand is the sense strand of the siRNA.

43. The method of any one of claims 29 to 42, wherein the oligonucleotide comprises at least one modified internucleotide linkage, optionally wherein the at least one modified internucleotide linkage is a phosphorothioate linkage.

44. The method of any one of claims 29-43, wherein the oligonucleotide comprises one or more modified nucleotides, optionally wherein the modified nucleotide comprises 2′-O-methoxyethylribose (MOE), locked nucleic acid (LNA), a 2′-fluoro modification, or a morpholino modification.

45. The method of any one of claims 29-44, wherein the oligonucleotide is 10-50 nucleotides in length, optionally 15-25 nucleotides in length.

46. The method of any one of claims 29-45, wherein the antibody is covalently linked to the 5′ of the oligonucleotide.

47. The method of any one of claims 29-46, wherein the antibody is covalently linked to the 3′ of the oligonucleotide.

48. The method of any one of claims 29-47, wherein the antibody is covalently linked to the oligonucleotide via a linker, optionally a Val-cit linker.

49. The method of any one of claims 29-48, wherein the complexes eluted in step (ii) comprise an antibody covalently linked to 1, 2, or 3 oligonucleotides.

50. The method of any one of claims 29-49, wherein the eluent obtained from step (ii) comprises undetectable levels of unlinked oligonucleotide.

51. The method of any one of claims 29-50, wherein the mixture in step (i) was isolated from a hydrophobic interaction chromatography resin prior to step (i).

52. A method of isolating a complex or plurality of complexes each comprising an antibody covalently linked to one or more oligonucleotides, the method comprising:
(i) contacting a first mixture comprising the complexes, unlinked antibodies, and unlinked oligonucleotides with a hydrophobic resin under conditions in which the complexes and the unlinked oligonucleotides but not the unlinked antibodies adsorb to the hydrophobic resin, thus separating the unlinked antibodies from the complexes and the unlinked oligonucleotides adsorbed to the hydrophobic resin; and
(ii) obtaining a second mixture comprising the complexes and the unlinked oligonucleotides by eluting the complexes and the unlinked oligonucleotides from the hydrophobic resin under conditions in which the complexes dissociate from the hydrophogic resin;
(iii) contacting the second mixture obtained in step (ii) with a mixed-mode resin that comprises positively-charged metal sites and negatively charged ionic sites, under conditions in which the complexes adsorb to the mixed-mode resin, and
(iv) eluting the complexes from the mixed-mode resin under conditions in which the complexes dissociate from the mixed-mode resin.

53. The method claim 52, wherein the hydrophobic resin comprises a hydrophobic moiety selected from butyl, t-butyl, phenyl, ether, amide, or propyl groups.

54. The method of claim 52 or claim 53, wherein the mixed-mode resin is an apatite resin, optionally wherein the apatite resin is a hydroxyapatite resin, a ceramic hydroxyapatite resin, a hydroxyfluoroapatite resin, a fluoroapatite resin, or a chlorapatite resin.

55. The method of any one of claims 52-54, wherein the conditions in step (i) comprise a conductivity of at least 70 mS/cm, and/or the conditions in step (ii) comprises a conductivity of 10-70 mS/cm.

56. The method of any one of claims 52-55, wherein the conditions in step (i) and or step (ii) are achieved using an anti-chaotropic salt, optionally wherein the anti-chaotropic salt is ammonium sulfate.

57. The method of any one of claims 52-56, wherein the hydrophobic resin is equilibrated prior to step (i), optionally equilibrated with a solution comprising at least 500 mM of ammonium sulfate.

58. The method of any one of claims 52-57, wherein the mixture in step (i) further comprises at least 500 mM of ammonium sulfate, optionally wherein the mixture in step (i) further comprises 500 mM-1 M of ammonium sulfate.

59. The method of any one of claims 52-58, further comprising washing the hydrophobic resin between step (i) and step (ii) with a solution comprising at least 500 mM of ammonium sulfate.

60. The method of any one of claims 52-59, wherein step (ii) comprises applying a first elution solution comprising up to 200 mM of chloride ions and up to 100 mM of ammonium sulfate to the hydrophobic resin to elute the complexes and the unlinked oligonucleotides, optionally wherein the first elution solution does not contain ammonium sulfate.

61. The method of claim 60, wherein the first elution solution is PBS, or comprises up to 25 mM chloride ions.

62. The method of any one of claims 52-59, wherein step (ii) comprises applying a gradually decreasing concentration of ammonium sulfate to the hydrophobic resin to elute the complexes and the unlinked oligonucleotides, optionally wherein the concentration of ammonium sulfate decreases from at least 500 mM to less than 100 mM and/or the gradually decreasing concentration of ammonium sulfate is applied over 5-12 column volumes (CVs), optionally 6-8 CVs.

63. The method of any one of claims 52-60, wherein the second mixture in step (iii) further comprises up to 20 mM phosphate ions and/or up to 30 mM chloride ions, optionally wherein the second mixture in step (iii) further comprises up to 10 mM phosphate ions and/or up to 25 mM chloride ions.

64. The method of claim 63, wherein the unlinked oligonucleotide does not adsorb to the mixed-mode resin in step (iii).

65. The method of any one of claims 52-63, wherein the second mixture in step (iii) further comprises up to 5 mM phosphate ions and/or up to 10 mM chloride ions, optionally wherein the second mixture in step (iii) further comprises up to 3 mM phosphate ions and/or up to 8 mM chloride ions.

66. The method of claim 65, wherein some or all of the unlinked oligonucleotide adsorb to the mixed-mode resin in step (iii).

67. The method of claim 65 or claim 66, further comprising washing the mixed-mode resin between step (iii) and step (iv) with a solution comprising up to 20 mM phosphate ions and/or up to 30 mM chloride ions to remove the unlinked oligonucleotide from the mixed mode resin, optionally wherein the solution comprises up to 10 mM phosphate ions and/or up to 25 mM chloride ions.

68. The method of any one of claims 52-67, wherein step (iv) comprises applying a second elution solution comprising at least 30 mM phosphate ions and/or at least 50 mM chloride ions to the mixed-mode resin to elute the complexes, optionally wherein the second elution solution comprises at least 100 mM phosphate ions and/or at least 100 mM chloride.

69. The method of any one of claims 52-68, wherein the antibody is a full length IgG, a Fab fragment, a Fab′ fragment, a F(ab′)2 fragment, a scFv, or a Fv fragment.

70. The method of claim 69, wherein the antibody is an anti-transferrin receptor antibody.

71. The method of any one of claims 52-70, wherein the oligonucleotide is single stranded.

72. The method of claim 71, wherein the oligonucleotide is an antisense oligonucleotide, optionally a gapmer or a phosphorodiamidate morpholino oligomer (PMO).

73. The method of claim 71, wherein the oligonucleotide is one strand of a double stranded oligonucleotide, optionally wherein the double stranded oligonucleotide is a siRNA, and optionally wherein the one strand is the sense strand of the siRNA.

74. The method of any one of claims 52-73, wherein the oligonucleotide comprises at least one modified internucleotide linkage, optionally wherein the at least one modified internucleotide linkage is a phosphorothioate linkage.

75. The method of any one of claims 52-74, wherein the oligonucleotide comprises one or more modified nucleotides, optionally wherein the modified nucleotide comprises 2′-O-methoxyethylribose (MOE), locked nucleic acid (LNA), a 2′-fluoro modification, or a morpholino modification.

76. The method of any one of claims 52-75, wherein the oligonucleotide is 10-50 nucleotides in length, optionally 15-25 nucleotides in length.

77. The method of any one of claims 52-76, wherein the antibody is covalently linked to the 5′ of the oligonucleotide.

78. The method of any one of claims 52-77, wherein the antibody is covalently linked to the 3′ of the oligonucleotide.

79. The method of any one of claims 52-78, wherein the antibody is covalently linked to the oligonucleotide via a linker, optionally a Val-cit linker.

80. The method of any one of claims 52-79, wherein the complexes eluted in step (iv) comprise an antibody covalently linked to 1, 2, or 3 oligonucleotides.

81. The method of any one of claims 52-80, wherein the eluent obtained from step (iv) comprises undetectable levels of unlinked oligonucleotide and/or undetectable levels of unlinked antibodies.

82. A method of processing complexes that comprise a protein covalently linked to one or more oligonucleotides, the method comprising:
(i) separating the complexes from unlinked oligonucleotides by contacting a mixture that comprises the complexes and the unlinked oligonucleotides with a mixed-mode resin that comprises positively-charged metal sites and negatively charged ionic sites, under conditions in which the complexes adsorb to the mixed-mode resin, and
(ii) eluting the unlinked oligonucleotide while the complexes remain adsorbed to the mixed-mode resin.

83. The method of claim 82, wherein the mixed-mode resin is an apatite resin.

84. The method of claim 83, wherein the apatite resin is a hydroxyapatite resin, a ceramic hydroxyapatite resin, a hydroxyfluoroapatite resin, a fluoroapatite resin, or a chlorapatite resin.

85. The method of any one of claims 82-84, wherein the mixture further comprises at least 1 mM phosphate ions and/or at least 5 mM chloride ions, optionally wherein the mixture further comprises 5 mM phosphate ions and 25 mM chloride ions.

86. The method of any one of claims 82-85, wherein the mixture was isolated from a hydrophobic interaction chromatographic resin prior to step (i).

87. The method of any one of claims 82-85, wherein the unlinked oligonucleotide is eluted in step (ii) by the addition of a wash solution to the mixed-mode resin, optionally wherein the wash solution comprises 1-50 mM phosphate ions and/or at least 5-50 mM chloride ions, optionally wherein the wash solution comprises 5 mM phosphate ions and 25 mM chloride ions.

88. The method of any one of claims 82-87, further comprising step (iii), following step (ii), eluting the plurality of complexes from the mixed-mode resin.

89. The method of claim 88, wherein the plurality of complexes are eluted in step (iii) by the addition of an eluent solution to the mixed-mode resin, optionally wherein the eluent solution comprises at least 5 mM phosphate ions and/or at least 5 mM chloride ions, optionally wherein the eluent solution comprises 100 mM phosphate ions and 100 mM chloride ions.

90. The method of any one of claims 82-89, wherein the protein is an antibody, optionally a muscle-targeting antibody.

91. The method of claim 90, wherein the muscle-targeting antibody specifically binds to an extracellular epitope of a transferrin receptor.

92. The method of claim 91, wherein the muscle-targeting antibody competes for specific binding to an epitope of a transferrin receptor with an antibody listed in Table 2.

93. The method of any one of claims 90-92, wherein the muscle-targeting antibody is in the form of a ScFv, a Fab fragment, Fab′ fragment, F(ab′)2 fragment, or Fv fragment.

94. The method of any one of claims 82-93, wherein the oligonucleotide comprises at least one modified internucleotide linkage, optionally wherein the at least one modified internucleotide linkage is a phosphorothioate linkage.

95. The method of any one of claims 82-94, wherein the oligonucleotide comprises one or more modified nucleotides.

96. The method of any one of claims 82-95, wherein the oligonucleotide is 10-50 nucleotides in length, optionally 15-25 nucleotides in length.

97. The method of any one of claims 82-96, wherein the oligonucleotide comprises a region of complementarity to gene listed in Table 1 or mRNA encoded therefrom.

98. The method of any one of claims 82-97, wherein the protein is linked to one, two, or three oligonucleotides.

99. The method of any one of claims 88-98, wherein the eluent obtained from step (iii) comprises undetectable levels of unlinked oligonucleotide.

100. The method of any one of claims 82-99, wherein the oligonucleotide is a single stranded oligonucleotide.

101. A method of processing complexes, wherein each complex comprises a protein covalently linked to one or more oligonucleotides, the method comprising:
(i) contacting a first mixture comprising the complexes, unlinked oligonucleotides, and unlinked proteins with a hydrophobic interaction chromatographic (HIC) resin, under conditions in which the complexes and unlinked oligonucleotides adsorb to the HIC resin;
(ii) eluting the unlinked protein from the HIC resin;
(iii) following step (ii), eluting from the HIC resin a second mixture comprising the complexes and unlinked oligonucleotides;
(iv) contacting the second mixture with an mixed-mode resin that comprises positively-charged metal sites and negatively charged ionic site, under conditions in which the complexes adsorb to the mixed-mode resin;
(v) eluting the unlinked oligonucleotide while the complexes remain adsorbed to the mixed-mode resin; and
(vi) following step (v), eluting the plurality of complexes from the mixed-mode resin.

102. The method of claim 101, wherein the HIC resin comprises butyl, t-butyl, methyl, and/or ethyl functional groups.

103. The method of either one of claims 101 or 102, wherein the HIC resin is equilibrated prior to step (i), optionally equilibrated with at least 500 mM ammonium sulfate.

104. The method of any one of claims 101 to 103, wherein the unlinked protein is eluted in step (ii) by the addition of a HIC wash solution to the HIC resin, optionally wherein the HIC wash solution comprises at least 500 mM ammonium sulfate.

105. The method of any one of claims 101 to 104, wherein the complexes and unlinked oligonucleotide are eluted from the HIC resin in step (iii) by the addition of a HIC eluent solution to the HIC resin, optionally wherein the HIC eluent solution comprises less than 100 mM phosphate ions and/or 100 mM chloride ions, optionally wherein the HIC eluent solution comprises 5 mM phosphate ions and 25 mM chloride ions.

106. The method of any one of claims 101 to 105, wherein the mixed-mode resin is an apatite resin.

107. The method of any one of claims 101 to 106, wherein the apatite resin is a hydroxyapatite resin, a ceramic hydroxyapatite resin, a hydroxyfluoroapatite resin, a fluoroapatite resin, or a chlorapatite resin.

108. The method of claim 107, wherein the second mixture further comprises at least 1 mM phosphate ions and/or at least 5 mM chloride ions, optionally wherein the mixture further comprises 5 mM phosphate ions and 25 mM chloride ions.

109. The method of any one of claims 101 to 108, wherein the unlinked oligonucleotide is eluted from the mixed-mode resin in step (v) by the addition of a mixed-mode wash solution to the mixed-mode resin, optionally wherein the mixed-mode wash solution comprises 1-50 mM phosphate ions and/or at least 5-50 mM chloride ions, optionally wherein the mixed-mode wash solution comprises 5 mM phosphate ions and 25 mM chloride ions.

110. The method of any one of claims 101 to 109, wherein the plurality of complexes is eluted in step (vi) by the addition of a mixed-mode eluent solution to the mixed-mode resin, optionally wherein the mixed-mode eluent solution comprises at least 20 mM phosphate ions and/or at least 10 mM chloride ions, optionally wherein the mixed-mode eluent solution comprises 100 mM phosphate ions and 100 mM chloride ions.

111. The method of any one of claims 101 to 110, wherein the protein is an antibody, optionally a muscle-targeting antibody.

112. The method of claim 111, wherein the muscle-targeting antibody specifically binds to an extracellular epitope of a transferrin receptor.

113. The method of claim 112, wherein the muscle-targeting antibody competes for specific binding to an epitope of a transferrin receptor with an antibody listed in Table 2.

114. The method of any one of claims 101 to 113, wherein the muscle-targeting antibody is in the form of a ScFv, a Fab fragment, Fab′ fragment, F(ab′)2 fragment, or Fv fragment.

115. The method of any one of claims 101 to 114, wherein the oligonucleotide comprises at least one modified internucleotide linkage, optionally wherein the at least one modified internucleotide linkage is a phosphorothioate linkage.

116. The method of any one of claims 101 to 115, wherein the oligonucleotide comprises one or more modified nucleotides.

117. The method of any one of claims 101 to 116, wherein the oligonucleotide is 10-50 nucleotides in length, optionally 15-25 nucleotides in length.

118. The method of any one of claims 101 to 117, wherein the oligonucleotide comprises a region of complementarity to gene listed in Table 1 or mRNA encoded therefrom.

119. The method of any one of claims 101 to 118, wherein the plurality of complexes of step (vi) comprise undetectable levels of unlinked protein and undetectable levels of unlinked oligonucleotide.

120. The method of any one of claims 101-119, wherein the oligonucleotide is a single stranded oligonucleotide.
